(19) United States
(12) Patent Application Publication
(10) Pub. No.: US 2025/0152190 A1
(43) Pub. Date: May 15, 2025

(54) SYSTEM AND METHOD FOR TISSUE INTERVENTION VIA IMAGE-GUIDED BOILING HISTOTRIPSY

(71) Applicant: Petal Surgical, Inc., Foster City, CA (US)

(72) Inventor: Prashant CHOPRA, Foster City, CA (US)

(73) Assignee: Petal Surgical, Inc., Foster City, CA (US)

(21) Appl. No.: 18/744,469

(22) Filed: Jun. 14, 2024

Related U.S. Application Data
(63) Continuation of application No. PCT/US2022/081891, filed on Dec. 16, 2022.
(60) Provisional application No. 63/356,988, filed on Jun. 29, 2022.
(60) Provisional application No. 63/308,051, filed on Feb. 8, 2022.
(60) Provisional application No. 63/290,647, filed on Dec. 16, 2021.

(51) Int. Cl. A61B 17/22; A61B 34/20; A61B 34/37; A61B 90/00

(52) U.S. Cl. CPC A61B 17/22004; A61B 34/37; A61B 2017/22008; A61B 2034/2059; A61B 2090/064

(57) ABSTRACT

One embodiment is directed to a minimally invasive system for treating a targeted tissue structure of a patient, comprising: an electromechanical support assembly having a proximal portion and a distal portion; a computing system operatively coupled to the electromechanical support assembly; and a HIFU treatment transducer array coupled to the distal portion of the electromechanical support assembly and operatively coupled to the computing system; wherein the computing system is configured to operate the electromechanical support assembly to control a position of the transducer assembly relative to the patient such that a treatment focus of the HIFU treatment transducer array is aligned to treat at least a portion of the targeted tissue structure of the patient, and to operate the HIFU treatment transducer array to controllably create a pulsatile wavefront of ultrasound radiation directed at the treatment focus, the pulsatile wavefront configured to produce one or more vapor bubbles within the targeted tissue structure and to controllably produce cavitation of the one of more vapor bubbles such that a controllably lysed portion of the targeted tissue structure is created.

### CROSS-REFERENCE TO RELATED APPLICATIONS

This application is a continuation of International Application No. PCT/US2022/081891, filed Dec. 16, 2022, which claims the benefit of U.S. Provisional Application No. 63/290,647, filed Dec. 16, 2021, U.S. Provisional Application No. 63/308,051, filed Feb. 8, 2022, and U.S. Provisional Application No. 63/356,988, filed Jun. 29, 2022, each of which is hereby incorporated by reference in its entirety.

### FIELD OF THE INVENTION

The present invention relates generally to systems and methods for modification of tissue structures for treatment of pathological conditions, and enhancement of non-pathological function, via the use of boiling histotripsy.

### BRIEF DESCRIPTION OF DRAWINGS

FIGS. 1A-1C illustrate various aspects of spine anatomy.

FIGS. 2A and 2B illustrate various aspects of spine anatomy with herniation issues.

FIGS. 3, 4A, and 4B illustrate aspects of conventional approaches to spine intervention.

FIGS. 5, 6A, 6B, 6C, 6D, 6E, and 7 illustrate aspects of histotripsy-based approaches for spine intervention utilizing one or more ultrasound transducers.

FIGS. 8A, 8B, 9A, 9B, and 9C illustrate various aspects of hardware configurations which may be utilized with an ultrasound-based histotripsy intervention.

FIGS. 10A, 10B, 10C, 10D, and 11 illustrate various aspects of configurations which may be utilized in an ultrasound-based histotripsy intervention which may also feature an electromechanical or robotic positioning and/or orientation system.

FIGS. 12A, 12B, 13, 14A, 14B, and 15 illustrate various aspects of configurations which may be utilized in an ultrasound-based histotripsy intervention which may also feature an electromechanical or robotic positioning and/or orientation system, as well as one or more alternative imaging modalities.

FIGS. 16A, 16B, 17A, 17B, and 18 illustrate various aspects of configurations which may be utilized to oscillate or sweep a field of view or imaging of a device such as an ultrasound transducer, as well as integrations of such configurations into interventional systems.

FIGS. 19, 20, 21, 22, 23, 24, 25, 26, 27A, 27B, and 28 illustrate various aspects of configurations which may be utilized in an ultrasound-based histotripsy intervention which may also feature one or more devices to assist in determining and/or tracking the position of various components relative to each other.

FIGS. 29A, 29B, and 30 illustrate various aspects of configurations for utilizing ultrasound-based histotripsy in a medical intervention pertaining to a cyst within a patient.

FIGS. 31A, 31B, 31C, 32, and 33 illustrate various aspects of configurations for utilizing ultrasound-based histotripsy in a medical intervention pertaining to facet joints and/or a ligamentum flavum within a patient.

FIGS. 34A, 34B, and 35 illustrate various aspects of configurations for utilizing ultrasound-based histotripsy in a medical intervention pertaining to an epidural tumor within a patient.

FIGS. 36A, 36B, and 37 illustrate various aspects of configurations for utilizing ultrasound-based histotripsy in a medical intervention pertaining to a metastatic spine tumor within a patient.

FIGS. 38A, 38B, and 39 illustrate various aspects of configurations for utilizing ultrasound-based histotripsy in a medical intervention pertaining to a sarcoma of the spine within a patient.

FIGS. 40A, 40B, and 41 illustrate various aspects of configurations for utilizing ultrasound-based histotripsy in a medical intervention pertaining to a myeloma of the spine within a patient.

FIGS. 42A, 42B, 42C, 43, 44, 45A, 45B, 45C, 45D, and 46 illustrate various aspects of configurations for utilizing ultrasound-based histotripsy in a medical intervention pertaining to joints and related tissue structures within a patient.

FIGS. 47A, 47B, and 48 illustrate various aspects of configurations for utilizing ultrasound-based histotripsy in a medical intervention pertaining to a targeted lymphoma lesion within a patient.

FIGS. 49A, 49B, 49C, 49D, and 50 illustrate various aspects of configurations for utilizing ultrasound-based histotripsy in a medical intervention pertaining to a gland within a patient, such as a prostate gland.

FIGS. 51A, 51B, 51C, 52, and 53 illustrate various aspects of configurations for utilizing ultrasound-based histotripsy in a medical intervention pertaining to tissue structures associated with a uterus within a patient, such as fibroid tumors or endometrial lesions.

FIGS. 54A, 54B, 55, 56A, 56B, and 57 illustrate various aspects of configurations for utilizing ultrasound-based histotripsy in a medical intervention pertaining to aspects of the cardiovascular system of a patient, such as plaques, clots, and/or embolisms.

FIGS. 58A-70 illustrate various aspects of systems or portions thereof for utilizing or facilitating ultrasound-based histotripsy interventions.

### BACKGROUND

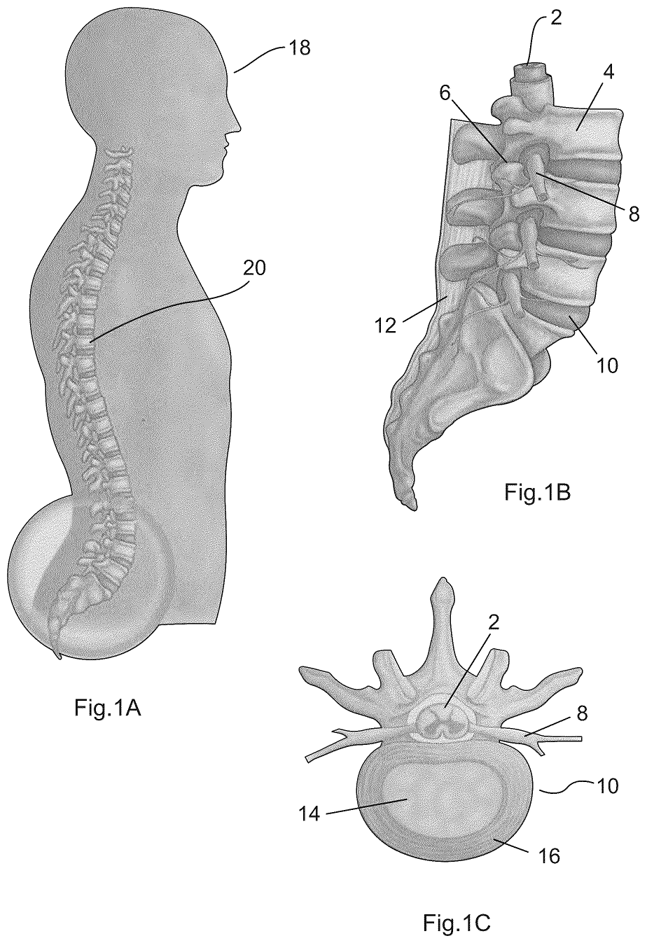

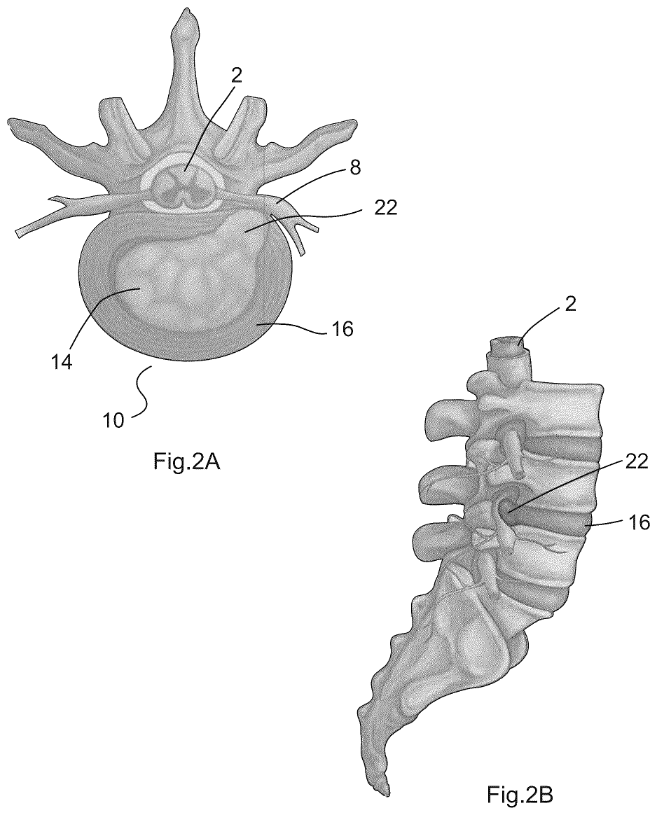

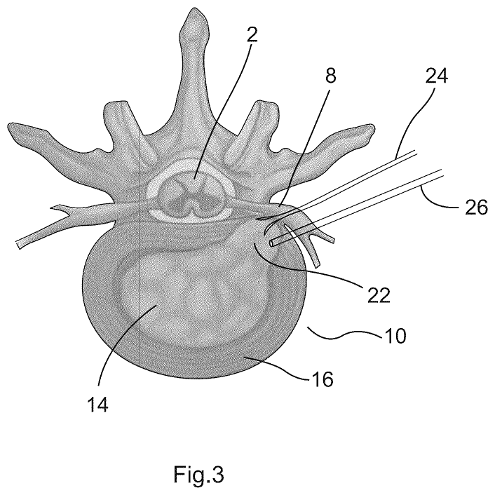

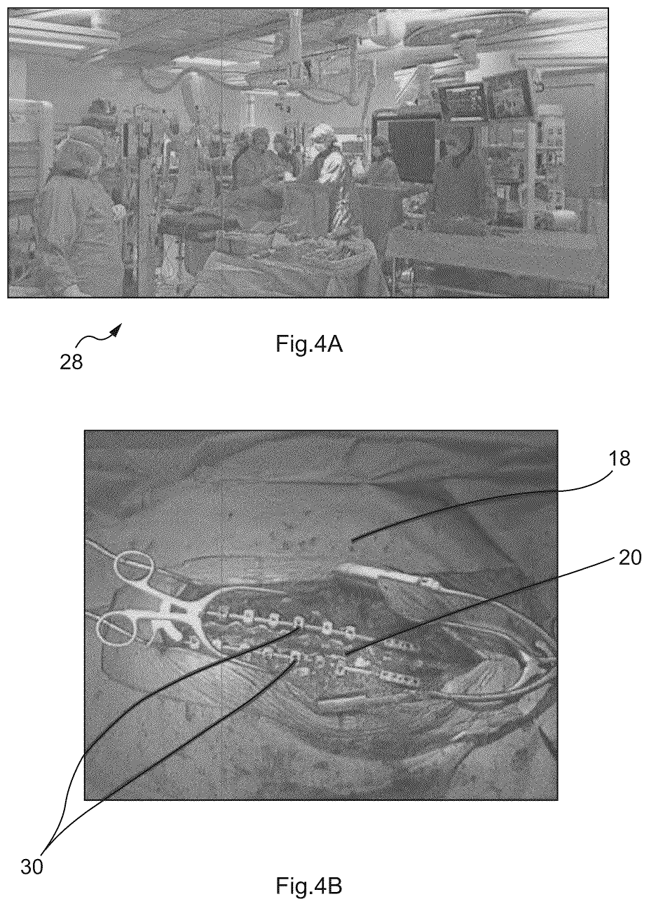

The prevalence of structural problems in tissues of the human is high and has been correlated with significant pain, disability, and generally costly intervention using techniques such as open surgery. For example, it is estimated that approximately forty percent of people over the age of forty have at least one variety of degenerative intervertebral disc disease (“DDD”) associated with their spine. Referring to FIG. 1A, the spine (20) of a patient (18) is shown. FIG. 1B illustrates aspects of the lower spine, such as the spinal cord (2), a series of vertebrae (4), facet joints (6) wherein aspects of the vertebral structures interface, spinal nerve (8) structures, and intervertebral discs (10). Referring to FIG. 1C, some patients, for example, suffer from pain and/or instability that may be associated with a herniation condition, wherein generally a portion of a nucleus pulposis (14) and/or annulus fibrosis (16) of an intervertebral disc (10) may be creating aberrant contact with a nearby portion of a nerve (8) associated with the spine of a patient. FIGS. 2A and 2B illustrate such a herniation scenario, wherein a herniation portion (22) of the nucleus pulposis (14) extends outward from the bounds of the annulus fibrosis (16) toward an associated nerve (8). Referring to FIG. 3, to address such condition, a spine surgery may be conducted to modify, decrease, or remove the aberrant contact condition between these structures; FIG. 3 illustrates the distal ends of two surgical tools (24, 26), such as a cutting grasper and an aspiration needle, being utilized to remove at least a portion of the herniation (22) through an invasive surgical procedure. Conventionally, such a spine surgery generally would be preceded by imaging the associated tissues using modalities such magnetic resonance, computed tomography, bi-plane radiography, fluoroscopy, ultrasound, and/or camera devices to preoperatively understand the scenario. One or more of such imaging modalities may be utilized intraoperatively as well, to assist interventional personnel in optimizing the treatment paradigm. For example, FIG. 4A illustrates a modern spine surgery operating room configuration (28), and FIG. 4B illustrates aspects of a surgical intervention on a patient's (18) spine (20). With the patient (18) in a prone position upon an operating table, incisions may be created on the patient's back to provide access to the subject tissue structures while one or more imaging modalities may be utilized to assist in understanding the positions and orientations of various anatomic structures and interventional tools as these tools are positioned and oriented to engage the subject tissue structures and, for example, remove a portion of nucleus pulposis tissue that has found its way beyond the usual bounds of the annulus fibrosis and into contact with a nearby nerve. It may be helpful in other interventional variations to implant various metallic and/or nonmetallic materials or structures (30) to assist in providing additional structural support for the subject region of the spine in the wake of such a tissue removal intervention. As noted above, generally such interventions are invasive. They also require significant resource, time, and money, which may be scarce in the various healthcare delivery systems at issue. There is a need for systems, methods, apparatuses, and configurations to address the need for efficient and minimally invasive tissue structure intervention to address challenges such as degenerative disc disease of the spine. More generally, there is a need for minimally invasive and efficient systems, methods, apparatuses, and configurations for conducting precision diagnostic imaging to assist in understanding positions and orientations of subject tissue structures and interventional tools in pre-operative, post-operative, and intraoperative scenarios. Further, there is a need for minimally invasive and efficient systems, methods, apparatuses, and configurations for facilitating controlled navigation of various instruments relative to subject tissue structures, and for modifying and/or removing targeted tissue structures or portions thereof.

### SUMMARY OF THE DISCLOSURE

One embodiment is directed to a minimally invasive system for treating a targeted tissue structure of a patient, comprising: an electromechanical support assembly having a proximal portion and a distal portion; a computing system operatively coupled to the electromechanical support assembly; and a HIFU treatment transducer array coupled to the distal portion of the electromechanical support assembly and operatively coupled to the computing system; wherein the computing system is configured to operate the electromechanical support assembly to control a position of the transducer assembly relative to the patient such that a treatment focus of the HIFU treatment transducer array is aligned to treat at least a portion of the targeted tissue structure of the patient, and to operate the HIFU treatment transducer array to controllably create a pulsatile wavefront of ultrasound radiation directed at the treatment focus, the pulsatile wavefront configured to produce one or more vapor bubbles within the targeted tissue structure and to controllably produce cavitation of the one of more vapor bubbles such that a controllably lysed portion of the targeted tissue structure is created. The electromechanical support assembly may comprise a plurality of elongate portions coupled by one or more movable joints. The one or more movable joints may be coupled to one or more encoders operatively coupled to the computing system and configured to provide inputs to the computing system for determining positions of the one or more movable joints. The electromechanical support assembly may comprise one or more sensors configured to sense one or more loads within the electromechanical support assembly associated with a physical interface between the HIFU treatment transducer array and the patient. The one or more sensors may be chosen from the group consisting of: a joint load sensor, a joint torque sensor, a strain gauge, and a deflection gauge. The system further may comprise one or more motors operatively coupled to the electromechanical support assembly and configured to apply loads thereto to maintain or change position or orientation of the electromechanical support assembly. The electromechanical support assembly may comprise a robotic arm. The computing system further may be configured to operate the electromechanical support assembly to control an orientation of the transducer assembly relative to the patient. The electromechanical support assembly may be controlled by the computer in response to inputs provided by an operator. The inputs provided by the operator may be manual electromechanical support assembly movement commands. The inputs provided by the operator may be commands for the electromechanical support assembly to follow a prescribed set of movements. The electromechanical support assembly may be controlled by the computer automatically in response to prescribed inputs provided by an operator. The HIFU treatment transducer array may be operatively coupled to the computing system using a wireless connectivity configuration. The HIFU treatment transducer array may be operatively coupled to the computing system using a wired connectivity configuration. The electromechanical support assembly may be operatively coupled to the computing system using a wireless connectivity configuration. The electromechanical support assembly may be operatively coupled to the computing system using a wired connectivity configuration. The system further may comprise an imaging ultrasound transducer having a ultrasound imaging field of view aligned to capture at least a portion of the treatment focus of the HIFU treatment transducer array. The HIFU treatment transducer array and the imaging ultrasound transducer may be both coupled to the distal portion of the electromechanical support assembly. The system further may comprise a delivery interface positioned between the HIFU treatment transducer array and the patient and configured to provide an efficient medium for conducting sound energy between the HIFU treatment transducer array and the patient. The system further may comprise a layer of acoustic gel interposed between the delivery interface and the patient and configured to further assist in efficient transmission between the HIFU treatment transducer array and the patient. The treatment focus may have a maximum dimension of about 5 millimeters. The treatment focus may have a maximum dimension of about 100 microns. The HIFU treatment transducer array may have an output frequency of between about 1 MHz and about 3 MHz. The HIFU treatment transducer array may have an output power of between about 300 watts and about 4,000 watts. The pulsatile wavefront may comprise a plurality of waves formed into a pulse, the pulse having a pulse duration of between about 1 and about 30 milliseconds. The computing system may be configured to produce the pulsatile wavefront for the pulse duration, followed by a pause of between about 0.1 second to about 1 second, before initiating another pulse. The waves may be configured to have a pressure amplitude received at the treatment focus of greater than about 60 MPa. The waves may be configured to have a negative peak pressure received at the treatment focus of between about 10 MPa and about 15 MPa. The controllably lysed portion may be created, at least in part, by an acoustic fountain reaction associated with the cavitation of the one or more vapor bubbles.

Another embodiment is directed to a minimally invasive system for treating a targeted tissue structure of a patient, comprising: an electromechanical support assembly having a proximal portion and a distal portion; a source of preoperative image data pertaining to the targeted tissue structure of the patient; a computing system operatively coupled to the electromechanical support assembly and the source of preoperative image data; a HIFU treatment transducer array coupled to the distal portion of the electromechanical support assembly and operatively coupled to the computing system; wherein the computing system is configured to operate the electromechanical support assembly to control a position of the HIFU treatment transducer assembly relative to the patient by registering coordinate systems of the HIFU treatment transducer assembly and patient relative to the preoperative image data from the source of preoperative image data, such that the preoperative image data may be utilized to assist in positioning the HIFU treatment transducer relative to anatomical features of the patient, the computing system being further configured such that a treatment focus of the HIFU treatment transducer array is aligned to treat at least a portion of the targeted tissue structure of the patient, and to operate the HIFU treatment transducer array to controllably create a pulsatile wavefront of ultrasound radiation directed at the treatment focus, the pulsatile wavefront configured to produce one or more vapor bubbles within the targeted tissue structure and to controllably produce cavitation of the one of more vapor bubbles such that a controllably lysed portion of the targeted tissue structure is created. The preoperative image data may be selected from the group consisting of: radiography data, fluoroscopy data, ultrasound imaging data, MRI data, and CT data. The system further may comprise a source of intraoperative data pertaining to the targeted tissue structure of the patient, wherein the intraoperative data also is co-registered with the preoperative image data, such that both the preoperative image data and the intraoperative image data may be utilized to assist in positioning the HIFU treatment transducer relative to anatomical features of the patient. The computing system may be configured to operate a neural network to assist in registering the coordinate systems of the HIFU treatment transducer assembly and patient relative to the preoperative image data from the source of preoperative image data. The computing system may be configured to operate a neural network to assist in registering the coordinate systems of the HIFU treatment transducer assembly and patient relative to the preoperative image data and the intraoperative data. The electromechanical support assembly may comprise a plurality of elongate portions coupled by one or more movable joints. The one or more movable joints may be coupled to one or more encoders operatively coupled to the computing system and configured to provide inputs to the computing system for determining positions of the one or more movable joints. The electromechanical support assembly may comprise one or more sensors configured to sense one or more loads within the electromechanical support assembly associated with a physical interface between the HIFU treatment transducer array and the patient. The one or more sensors may be chosen from the group consisting of: a joint load sensor, a joint torque sensor, a strain gauge, and a deflection gauge. The system further may comprise one or more motors operatively coupled to the electromechanical support assembly and configured to apply loads thereto to maintain or change position or orientation of the electromechanical support assembly. The electromechanical support assembly may comprise a robotic arm. The computing system further may be configured to operate the electromechanical support assembly to control an orientation of the transducer assembly relative to the patient. The electromechanical support assembly may be controlled by the computer in response to inputs provided by an operator. The inputs provided by the operator may be manual electromechanical support assembly movement commands. The inputs provided by the operator may be commands for the electromechanical support assembly to follow a prescribed set of movements. The electromechanical support assembly may be controlled by the computer automatically in response to prescribed inputs provided by an operator. The HIFU treatment transducer array may be operatively coupled to the computing system using a wireless connectivity configuration. The HIFU treatment transducer array may be operatively coupled to the computing system using a wired connectivity configuration. The electromechanical support assembly may be operatively coupled to the computing system using a wireless connectivity configuration. The electromechanical support assembly may be operatively coupled to the computing system using a wired connectivity configuration. The system further may comprise an imaging ultrasound transducer having a ultrasound imaging field of view aligned to capture at least a portion of the treatment focus of the HIFU treatment transducer array. The HIFU treatment transducer array and the imaging ultrasound transducer may be both coupled to the distal portion of the electromechanical support assembly. The system further may comprise a delivery interface positioned between the HIFU treatment transducer array and the patient and configured to provide an efficient medium for conducting sound energy between the HIFU treatment transducer array and the patient. The system further may comprise a layer of acoustic gel interposed between the delivery interface and the patient and configured to further assist in efficient transmission between the HIFU treatment transducer array and the patient. The treatment focus may have a maximum dimension of about 5 millimeters. The treatment focus may have a maximum dimension of about 100 microns. The HIFU treatment transducer array may have an output frequency of between about 1 MHz and about 3 MHz. The HIFU treatment transducer array may have an output power of between about 300 watts and about 4,000 watts. The pulsatile wavefront may comprise a plurality of waves formed into a pulse, the pulse having a pulse duration of between about 1 and about 30 milliseconds. The computing system may be configured to produce the pulsatile wavefront for the pulse duration, followed by a pause of between about 0.1 second to about 1 second, before initiating another pulse. The waves may be configured to have a pressure amplitude received at the treatment focus of greater than about 60 MPa. The waves may be configured to have a negative peak pressure received at the treatment focus of between about 10 MPa and about 15 MPa. The controllably lysed portion may be created, at least in part, by an acoustic fountain reaction associated with the cavitation of the one or more vapor bubbles. The system further may comprise one or more sensors operatively coupled to the computing system and configured to provide data to the computing system to allow for three-dimensional tracking of the electromechanical support structure relative to the patient. The one or more sensors may be selected from the group consisting of: a joint position sensor, an image capture device, an electromagnetic tracking sensor, a LIDAR device, an IMU, and an elongation sensor. The system further may comprise one or more sensors operatively coupled to the computing system and configured to provide data to the computing system to allow for three-dimensional tracking of the HIFU treatment transducer array relative to the patient. The one or more sensors may be selected from the group consisting of: a joint position sensor, an image capture device, an electromagnetic tracking sensor, a LIDAR device, an IMU, and an elongation sensor. The system further may comprise one or more sensors operatively coupled to the computing system and configured to provide data to the computing system to allow for three-dimensional tracking of the patient. The one or more sensors may be selected from the group consisting of: a joint position sensor, an image capture device, an electromagnetic tracking sensor, a LIDAR device, an IMU, and an elongation sensor.

Another embodiment is directed to a system for positioning instrumentation for a minimally invasive intervention on a patient, comprising: an elongate guiding member having a proximal end, a distal end, and a guiding lumen defined therethrough, the distal end being configured to be positioned adjacent to a targeted intervention location within the patient; an imaging transducer configured to be interfaced against the patient, the imaging transducer defining an imaging field of view which may be displayed upon an operatively coupled display device; wherein the imaging transducer is movably coupled to the elongate guiding member such that the field of view of the imaging transducer may be repositioned as the elongate guiding member is repositioned relative to the patient, such that the distal end of the elongate guiding member may be maintained within the field of view of the imaging transducer. The imaging transducer may be rotatably coupled to the elongate guiding member. The rotatable coupling may comprise a drive motor configured to produce oscillatory motion of at least a portion of the imaging transducer such that the field of view of the imaging transducer is swept in a pattern selected to capture the distal end of the elongate guiding member along with aspects of the patient adjacent the distal end of the elongate guiding member. The elongate guiding member may be an instrument selected from the group consisting of: a cannula, a needle, and a catheter. The elongate guiding member may be a needle configured to aspirate portions of tissue which may have been previously lysed in the targeted intervention location. The system further may comprise a HIFU treatment transducer array operatively coupled to a computing system, wherein the computing system is configured to position a treatment focus of the HIFU treatment transducer array in alignment to treat at least a portion of the targeted intervention location of the patient, and to operate the HIFU treatment transducer array to controllably create a pulsatile wavefront of ultrasound radiation directed at the treatment focus, the pulsatile wavefront configured to produce one or more vapor bubbles within the targeted intervention location and to controllably produce cavitation of the one of more vapor bubbles such that a controllably lysed portion of the tissue of the patient at the targeted intervention location is created. The system further may comprise a delivery interface positioned between the HIFU treatment transducer array and the patient and configured to provide an efficient medium for conducting sound energy between the HIFU treatment transducer array and the patient. The system further may comprise a layer of acoustic gel interposed between the delivery interface and the patient and configured to further assist in efficient transmission between the HIFU treatment transducer array and the patient. The treatment focus may have a maximum dimension of about 5 millimeters. The treatment focus may have a maximum dimension of about 100 microns. The HIFU treatment transducer array may have an output frequency of between about 1 MHz and about 3 MHz. The HIFU treatment transducer array may have an output power of between about 300 watts and about 4,000 watts. The pulsatile wavefront may comprise a plurality of waves formed into a pulse, the pulse having a pulse duration of between about 1 and about 30 milliseconds. The computing system may be configured to produce the pulsatile wavefront for the pulse duration, followed by a pause of between about 0.1 second to about 1 second, before initiating another pulse. The waves may be configured to have a pressure amplitude received at the treatment focus of greater than about 60 MPa. The waves may be configured to have a negative peak pressure received at the treatment focus of between about 10 MPa and about 15 MPa. The controllably lysed portion may be created, at least in part, by an acoustic fountain reaction associated with the cavitation of the one or more vapor bubbles.

Another embodiment is directed to a minimally invasive system for treating a targeted tissue structure of a patient, comprising: an electromechanical support assembly having a proximal portion and a distal portion; a computing system operatively coupled to the electromechanical support assembly; a HIFU treatment transducer array coupled to the distal portion of the electromechanical support assembly and operatively coupled to the computing system; an elongate guiding member movably coupled to the HIFU treatment transducer array and having a proximal end, a distal end, and a guiding lumen defined therethrough, the distal end being configured to be positioned adjacent to the targeted tissue structure within the patient; wherein the computing system is configured to operate the electromechanical support assembly to control a position of the transducer assembly relative to the patient such that a treatment focus of the HIFU treatment transducer array is aligned to treat at least a portion of the targeted tissue structure of the patient, and to operate the HIFU treatment transducer array to controllably create a pulsatile wavefront of ultrasound radiation directed at the treatment focus, the pulsatile wavefront configured to produce one or more vapor bubbles within the targeted tissue structure and to controllably produce cavitation of the one of more vapor bubbles such that a controllably lysed portion of the targeted tissue structure is created; and wherein the elongate guiding member is configured to be utilized to remove the controllably lysed portion.

The elongate guiding member may be movably coupled relative to the treatment focus of the HIFU treatment transducer array such that the distal portion of the elongate guiding member may be inserted along a predetermined axis selected to be aligned with the position of the treatment focus and controllably lysed portion. The elongate guiding member may be an instrument selected from the group consisting of: a cannula, a needle, and a catheter. The elongate guiding member may be a needle configured to aspirate the controllably lysed portion. The electromechanical support assembly may comprise a plurality of elongate portions coupled by one or more movable joints. The one or more movable joints may be coupled to one or more encoders operatively coupled to the computing system and configured to provide inputs to the computing system for determining positions of the one or more movable joints. The electromechanical support assembly may comprise one or more sensors configured to sense one or more loads within the electromechanical support assembly associated with a physical interface between the HIFU treatment transducer array and the patient. The one or more sensors may be chosen from the group consisting of: a joint load sensor, a joint torque sensor, a strain gauge, and a deflection gauge. The system further may comprise one or more motors operatively coupled to the electromechanical support assembly and configured to apply loads thereto to maintain or change position or orientation of the electromechanical support assembly. The electromechanical support assembly may comprise a robotic arm. The computing system may be further configured to operate the electromechanical support assembly to control an orientation of the transducer assembly relative to the patient. The electromechanical support assembly may be controlled by the computer in response to inputs provided by an operator. The inputs provided by the operator may be manual electromechanical support assembly movement commands. The inputs provided by the operator may be commands for the electromechanical support assembly to follow a prescribed set of movements. The electromechanical support assembly may be controlled by the computer automatically in response to prescribed inputs provided by an operator. The HIFU treatment transducer array may be operatively coupled to the computing system using a wireless connectivity configuration. The HIFU treatment transducer array may be operatively coupled to the computing system using a wired connectivity configuration. The electromechanical support assembly may be operatively coupled to the computing system using a wireless connectivity configuration. The electromechanical support assembly may be operatively coupled to the computing system using a wired connectivity configuration. The system further may comprise an imaging ultrasound transducer having a ultrasound imaging field of view aligned to capture at least a portion of the treatment focus of the HIFU treatment transducer array. The HIFU treatment transducer array and the imaging ultrasound transducer may be both coupled to the distal portion of the electromechanical support assembly. The system further may comprise a delivery interface positioned between the HIFU treatment transducer array and the patient and configured to provide an efficient medium for conducting sound energy between the HIFU treatment transducer array and the patient. The system further may comprise a layer of acoustic gel interposed between the delivery interface and the patient and configured to further assist in efficient transmission between the HIFU treatment transducer array and the patient. The treatment focus may have a maximum dimension of about 5 millimeters. The treatment focus may have a maximum dimension of about 100 microns. The HIFU treatment transducer array may have an output frequency of between about 1 MHz and about 3 MHz. The HIFU treatment transducer array may have an output power of between about 300 watts and about 4,000 watts. The pulsatile wavefront may comprise a plurality of waves formed into a pulse, the pulse having a pulse duration of between about 1 and about 30 milliseconds. The computing system may be configured to produce the pulsatile wavefront for the pulse duration, followed by a pause of between about 0.1 second to about 1 second, before initiating another pulse. The waves may be configured to have a pressure amplitude received at the treatment focus of greater than about 60 MPa. The waves may be configured to have a negative peak pressure received at the treatment focus of between about 10 MPa and about 15 MPa. The controllably lysed portion may be created, at least in part, by an acoustic fountain reaction associated with the cavitation of the one or more vapor bubbles.

Another embodiment is directed to a minimally invasive system for treating a targeted tissue structure of a patient, comprising: an electromechanical support assembly having a proximal portion and a distal portion; a computing system operatively coupled to the electromechanical support assembly; a HIFU treatment transducer array coupled to the distal portion of the electromechanical support assembly and operatively coupled to the computing system; wherein the computing system is configured to operate the electromechanical support assembly to control a position of the transducer assembly relative to the patient such that an interfacial load between the transducer assembly and the patient is controlled, and such that a treatment focus of the HIFU treatment transducer array is aligned to treat at least a portion of the targeted tissue structure of the patient, and to operate the HIFU treatment transducer array to controllably create a pulsatile wavefront of ultrasound radiation directed at the treatment focus, the pulsatile wavefront configured to produce one or more vapor bubbles within the targeted tissue structure and to controllably produce cavitation of the one of more vapor bubbles such that a controllably lysed portion of the targeted tissue structure is created. The electromechanical support assembly may comprise a plurality of elongate portions coupled by one or more movable joints. The one or more movable joints may be coupled to one or more encoders operatively coupled to the computing system and configured to provide inputs to the computing system for determining positions of the one or more movable joints. The electromechanical support assembly may comprise one or more sensors configured to sense one or more loads within the electromechanical support assembly associated with the interfacial load between the HIFU treatment transducer array and the patient. The computing system may be configured to maintain the interfacial load below a predetermined maximum. The computing system may be configured to maintain the interfacial load above a predetermined minimum and below a predetermined maximum during a period of treatment of the patient. The computing system may be configured to maintain a relative orientation between the HIFU treatment transducer and a most immediately adjacent portion of the patient's body while also maintaining the interfacial load above a predetermined minimum and below a predetermined maximum during a period of treatment of the patient. The computing system may be configured to facilitate a repositioning of the HIFU treatment transducer relative to the patient while also maintaining the relative orientation between the HIFU treatment transducer and most immediately adjacent portion of the patient's body, as well as also maintaining the interfacial load above a predetermined minimum and below a predetermined maximum during a period of treatment of the patient. The one or more sensors may be chosen from the group consisting of: a joint load sensor, a joint torque sensor, a strain gauge, and a deflection gauge. The system further may comprise one or more motors operatively coupled to the electromechanical support assembly and configured to apply loads thereto to maintain or change position or orientation of the electromechanical support assembly. The electromechanical support assembly may comprise a robotic arm. The computing system may be further configured to operate the electromechanical support assembly to control an orientation of the transducer assembly relative to the patient. The electromechanical support assembly may be controlled by the computer in response to inputs provided by an operator. The inputs provided by the operator may be manual electromechanical support assembly movement commands. The inputs provided by the operator may be commands for the electromechanical support assembly to follow a prescribed set of movements. The electromechanical support assembly may be controlled by the computer automatically in response to prescribed inputs provided by an operator. The HIFU treatment transducer array may be operatively coupled to the computing system using a wireless connectivity configuration. The HIFU treatment transducer array may be operatively coupled to the computing system using a wired connectivity configuration. The electromechanical support assembly may be operatively coupled to the computing system using a wireless connectivity configuration. The electromechanical support assembly may be operatively coupled to the computing system using a wired connectivity configuration. The system further may comprise an imaging ultrasound transducer having a ultrasound imaging field of view aligned to capture at least a portion of the treatment focus of the HIFU treatment transducer array. The HIFU treatment transducer array and the imaging ultrasound transducer may be both coupled to the distal portion of the electromechanical support assembly. The system further may comprise a delivery interface positioned between the HIFU treatment transducer array and the patient and configured to provide an efficient medium for conducting sound energy between the HIFU treatment transducer array and the patient. The system further may comprise a layer of acoustic gel interposed between the delivery interface and the patient and configured to further assist in efficient transmission between the HIFU treatment transducer array and the patient. The treatment focus may have a maximum dimension of about 5 millimeters. The treatment focus may have a maximum dimension of about 100 microns. The HIFU treatment transducer array may have an output frequency of between about 1 MHz and about 3 MHz. The HIFU treatment transducer array may have an output power of between about 300 watts and about 4,000 watts. The pulsatile wavefront may comprise a plurality of waves formed into a pulse, the pulse having a pulse duration of between about 1 and about 30 milliseconds. The computing system may be configured to produce the pulsatile wavefront for the pulse duration, followed by a pause of between about 0.1 second to about 1 second, before initiating another pulse. The waves may be configured to have a pressure amplitude received at the treatment focus of greater than about 60 MPa. The waves may be configured to have a negative peak pressure received at the treatment focus of between about 10 MPa and about 15 MPa. The controllably lysed portion may be created, at least in part, by an acoustic fountain reaction associated with the cavitation of the one or more vapor bubbles.

Another embodiment is directed to a robotic medical intervention system for treating a targeted tissue structure of a patient, comprising: a robotic system base; a computing system operatively coupled to the robotic system base; a plurality of robotic arms, each having a proximal and a distal end, the proximal ends being movably coupled to the robotic system base; a plurality of interventional end effectors, each interventional end effector coupled to the distal end of one of the plurality of robotic arm distal ends; wherein at least one of the intervention end effectors comprises a HIFU treatment transducer array, and wherein the computing system is configured to operate one of the plurality of robotic arms to control a position of the transducer assembly relative to the patient such that a treatment focus of the HIFU treatment transducer array is aligned to treat at least a portion of the targeted tissue structure of the patient, and to operate the HIFU treatment transducer array to controllably create a pulsatile wavefront of ultrasound radiation directed at the treatment focus, the pulsatile wavefront configured to produce one or more vapor bubbles within the targeted tissue structure and to controllably produce cavitation of the one of more vapor bubbles such that a controllably lysed portion of the targeted tissue structure is created. The computing system further may be configured to operate one of the plurality of robotic arms to control the position of the transducer assembly relative to the patient such that an interfacial load between the transducer assembly and the patient is controlled. At least one of the plurality or robotic arms may comprise a plurality of elongate portions coupled by one or more movable joints. The one or more movable joints may be coupled to one or more encoders operatively coupled to the computing system and configured to provide inputs to the computing system for determining positions of the one or more movable joints. The plurality of robotic arms may comprise one or more sensors configured to sense one or more loads associated with the interfacial load between the HIFU treatment transducer array and the patient. The computing system may be configured to maintain the interfacial load below a predetermined maximum. The computing system may be configured to maintain the interfacial load above a predetermined minimum and below a predetermined maximum during a period of treatment of the patient. The computing system may be configured to maintain a relative orientation between the HIFU treatment transducer and a most immediately adjacent portion of the patient's body while also maintaining the interfacial load above a predetermined minimum and below a predetermined maximum during a period of treatment of the patient. The computing system may be configured to facilitate a repositioning of the HIFU treatment transducer relative to the patient while also maintaining the relative orientation between the HIFU treatment transducer and most immediately adjacent portion of the patient's body, as well as also maintaining the interfacial load above a predetermined minimum and below a predetermined maximum during a period of treatment of the patient. The one or more sensors are chosen from the group consisting of: a joint load sensor, a joint torque sensor, a strain gauge, and a deflection gauge. The computing system may be further configured to operate at least one of the plurality of robotic arms to control an orientation of the transducer assembly relative to the patient. The position of the HIFU treatment transducer may be controlled by the computer in response to inputs provided by an operator. The inputs provided by the operator may be manual HIFU treatment transducer movement commands. The inputs provided by the operator may be commands for the HIFU treatment transducer to follow a prescribed set of movements. The position of the HIFU treatment transducer may be controlled by the computer automatically in response to prescribed inputs provided by an operator. The HIFU treatment transducer array may be operatively coupled to the computing system using a wireless connectivity configuration. The HIFU treatment transducer array may be operatively coupled to the computing system using a wired connectivity configuration. The plurality of robotic arms may be operatively coupled to the computing system using a wireless connectivity configuration. The plurality of robotic arms may be operatively coupled to the computing system using a wired connectivity configuration. The system further may comprise an imaging ultrasound transducer having a ultrasound imaging field of view aligned to capture at least a portion of the treatment focus of the HIFU treatment transducer array. The HIFU treatment transducer array and the imaging ultrasound transducer may be both coupled to the distal portion of at least one of the robotic arms. The system further may comprise a delivery interface positioned between the HIFU treatment transducer array and the patient and configured to provide an efficient medium for conducting sound energy between the HIFU treatment transducer array and the patient. The system further may comprise a layer of acoustic gel interposed between the delivery interface and the patient and configured to further assist in efficient transmission between the HIFU treatment transducer array and the patient. The treatment focus may have a maximum dimension of about 5 millimeters. The treatment focus may have a maximum dimension of about 100 microns. The HIFU treatment transducer array may have an output frequency of between about 1 MHz and about 3 MHz. The HIFU treatment transducer array may have an output power of between about 300 watts and about 4,000 watts. The pulsatile wavefront may comprise a plurality of waves formed into a pulse, the pulse having a pulse duration of between about 1 and about 30 milliseconds. The computing system may be configured to produce the pulsatile wavefront for the pulse duration, followed by a pause of between about 0.1 second to about 1 second, before initiating another pulse. The waves may be configured to have a pressure amplitude received at the treatment focus of greater than about 60 MPa. The waves may be configured to have a negative peak pressure received at the treatment focus of between about 10 MPa and about 15 MPa. The controllably lysed portion may be created, at least in part, by an acoustic fountain reaction associated with the cavitation of the one or more vapor bubbles.

Another embodiment is directed to a minimally invasive method for treating a targeted tissue structure of a patient, comprising: providing an electromechanical support assembly having a proximal portion and a distal portion, a computing system operatively coupled to the electromechanical support assembly, and a HIFU treatment transducer array coupled to the distal portion of the electromechanical support assembly and operatively coupled to the computing system; and utilizing the computing system to operate the electromechanical support assembly to control a position of the transducer assembly relative to the patient such that a treatment focus of the HIFU treatment transducer array is aligned to treat at least a portion of the targeted tissue structure of the patient, and to operate the HIFU treatment transducer array to controllably create a pulsatile wavefront of ultrasound radiation directed at the treatment focus, the pulsatile wavefront configured to produce one or more vapor bubbles within the targeted tissue structure and to controllably produce cavitation of the one of more vapor bubbles such that a controllably lysed portion of the targeted tissue structure is created. The electromechanical support assembly may comprise a plurality of elongate portions coupled by one or more movable joints. The one or more movable joints may be coupled to one or more encoders operatively coupled to the computing system and configured to provide inputs to the computing system for determining positions of the one or more movable joints. The electromechanical support assembly may comprise one or more sensors configured to sense one or more loads within the electromechanical support assembly associated with a physical interface between the HIFU treatment transducer array and the patient. The one or more sensors may be chosen from the group consisting of: a joint load sensor, a joint torque sensor, a strain gauge, and a deflection gauge. The method further may comprise providing one or more motors operatively coupled to the electromechanical support assembly and configured to apply loads thereto to maintain or change position or orientation of the electromechanical support assembly. The electromechanical support assembly may comprise a robotic arm. The computing system further may be configured to operate the electromechanical support assembly to control an orientation of the transducer assembly relative to the patient. The electromechanical support assembly may be controlled by the computer in response to inputs provided by an operator. The inputs provided by the operator may be manual electromechanical support assembly movement commands. The inputs provided by the operator may be commands for the electromechanical support assembly to follow a prescribed set of movements. The electromechanical support assembly may be controlled by the computer automatically in response to prescribed inputs provided by an operator. The HIFU treatment transducer array may be operatively coupled to the computing system using a wireless connectivity configuration. The HIFU treatment transducer array may be operatively coupled to the computing system using a wired connectivity configuration. The electromechanical support assembly may be operatively coupled to the computing system using a wireless connectivity configuration. The electromechanical support assembly may be operatively coupled to the computing system using a wired connectivity configuration. The method further may comprise providing an imaging ultrasound transducer having a ultrasound imaging field of view aligned to capture at least a portion of the treatment focus of the HIFU treatment transducer array. The HIFU treatment transducer array and the imaging ultrasound transducer may be both coupled to the distal portion of the electromechanical support assembly. The method further may comprise providing a delivery interface positioned between the HIFU treatment transducer array and the patient and configured to provide an efficient medium for conducting sound energy between the HIFU treatment transducer array and the patient. The method further may comprise providing a layer of acoustic gel interposed between the delivery interface and the patient and configured to further assist in efficient transmission between the HIFU treatment transducer array and the patient. The treatment focus may have a maximum dimension of about 5 millimeters. The treatment focus may have a maximum dimension of about 100 microns. The HIFU treatment transducer array may have an output frequency of between about 1 MHz and about 3 MHz. The HIFU treatment transducer array may have an output power of between about 300 watts and about 4,000 watts. The pulsatile wavefront may comprise a plurality of waves formed into a pulse, the pulse having a pulse duration of between about 1 and about 30 milliseconds. The computing system may be configured to produce the pulsatile wavefront for the pulse duration, followed by a pause of between about 0.1 second to about 1 second, before initiating another pulse. The waves may be configured to have a pressure amplitude received at the treatment focus of greater than about 60 MPa. The waves may be configured to have a negative peak pressure received at the treatment focus of between about 10 MPa and about 15 MPa. The controllably lysed portion may be created, at least in part, by an acoustic fountain reaction associated with the cavitation of the one or more vapor bubbles.

Another embodiment is directed to a minimally invasive method for treating a targeted tissue structure of a patient, comprising: providing an electromechanical support assembly having a proximal portion and a distal portion, a source of preoperative image data pertaining to the targeted tissue structure of the patient, a computing system operatively coupled to the electromechanical support assembly and the source of preoperative image data, and a HIFU treatment transducer array coupled to the distal portion of the electromechanical support assembly and operatively coupled to the computing system; utilizing the computing system to operate the electromechanical support assembly to control a position of the HIFU treatment transducer assembly relative to the patient by registering coordinate systems of the HIFU treatment transducer assembly and patient relative to the preoperative image data from the source of preoperative image data, such that the preoperative image data may be utilized to assist in positioning the HIFU treatment transducer relative to anatomical features of the patient; and utilizing the computing system to operate the HIFU treatment transducer array such that a treatment focus of the HIFU treatment transducer array is aligned to treat at least a portion of the targeted tissue structure of the patient, and such that the HIFU treatment transducer array controllably creates a pulsatile wavefront of ultrasound radiation directed at the treatment focus, the pulsatile wavefront configured to produce one or more vapor bubbles within the targeted tissue structure and to controllably produce cavitation of the one of more vapor bubbles such that a controllably lysed portion of the targeted tissue structure is created. The preoperative image data may be selected from the group consisting of: radiography data, fluoroscopy data, ultrasound imaging data, MRI data, and CT data. The method further may comprise providing a source of intraoperative data pertaining to the targeted tissue structure of the patient, wherein the intraoperative data also is co-registered with the preoperative image data, such that both the preoperative image data and the intraoperative image data may be utilized to assist in positioning the HIFU treatment transducer relative to anatomical features of the patient. The computing system may be configured to operate a neural network to assist in registering the coordinate systems of the HIFU treatment transducer assembly and patient relative to the preoperative image data from the source of preoperative image data. The computing system may be configured to operate a neural network to assist in registering the coordinate systems of the HIFU treatment transducer assembly and patient relative to the preoperative image data and the intraoperative data. The electromechanical support assembly may comprise a plurality of elongate portions coupled by one or more movable joints. The one or more movable joints may be coupled to one or more encoders operatively coupled to the computing system and configured to provide inputs to the computing system for determining positions of the one or more movable joints. The electromechanical support assembly may comprise one or more sensors configured to sense one or more loads within the electromechanical support assembly associated with a physical interface between the HIFU treatment transducer array and the patient. The one or more sensors may be chosen from the group consisting of: a joint load sensor, a joint torque sensor, a strain gauge, and a deflection gauge. The method further may comprise providing one or more motors operatively coupled to the electromechanical support assembly and configured to apply loads thereto to maintain or change position or orientation of the electromechanical support assembly. The electromechanical support assembly may comprise a robotic arm. The computing system further may be configured to operate the electromechanical support assembly to control an orientation of the transducer assembly relative to the patient. The electromechanical support assembly may be controlled by the computer in response to inputs provided by an operator. The inputs provided by the operator may be manual electromechanical support assembly movement commands. The inputs provided by the operator may be commands for the electromechanical support assembly to follow a prescribed set of movements. The electromechanical support assembly may be controlled by the computer automatically in response to prescribed inputs provided by an operator. The HIFU treatment transducer array may be operatively coupled to the computing system using a wireless connectivity configuration. The HIFU treatment transducer array may be operatively coupled to the computing system using a wired connectivity configuration. The electromechanical support assembly may be operatively coupled to the computing system using a wireless connectivity configuration. The electromechanical support assembly may be operatively coupled to the computing system using a wired connectivity configuration. The method further may comprise providing an imaging ultrasound transducer having a ultrasound imaging field of view aligned to capture at least a portion of the treatment focus of the HIFU treatment transducer array. The HIFU treatment transducer array and the imaging ultrasound transducer may be both coupled to the distal portion of the electromechanical support assembly. The method further may comprise providing a delivery interface positioned between the HIFU treatment transducer array and the patient and configured to provide an efficient medium for conducting sound energy between the HIFU treatment transducer array and the patient. The method further may comprise providing a layer of acoustic gel interposed between the delivery interface and the patient and configured to further assist in efficient transmission between the HIFU treatment transducer array and the patient. The treatment focus may have a maximum dimension of about 5 millimeters. The treatment focus may have a maximum dimension of about 100 microns. The HIFU treatment transducer array may have an output frequency of between about 1 MHz and about 3 MHz. The HIFU treatment transducer array may have an output power of between about 300 watts and about 4,000 watts. The pulsatile wavefront may comprise a plurality of waves formed into a pulse, the pulse having a pulse duration of between about 1 and about 30 milliseconds. The computing system may be configured to produce the pulsatile wavefront for the pulse duration, followed by a pause of between about 0.1 second to about 1 second, before initiating another pulse. The waves may be configured to have a pressure amplitude received at the treatment focus of greater than about 60 MPa. The waves may be configured to have a negative peak pressure received at the treatment focus of between about 10 MPa and about 15 MPa. The controllably lysed portion may be created, at least in part, by an acoustic fountain reaction associated with the cavitation of the one or more vapor bubbles. The method further may comprise providing one or more sensors operatively coupled to the computing system and configured to provide data to the computing system to allow for three-dimensional tracking of the electromechanical support structure relative to the patient. The one or more sensors may be selected from the group consisting of: a joint position sensor, an image capture device, an electromagnetic tracking sensor, a LIDAR device, an IMU, and an elongation sensor. The method further may comprise providing one or more sensors operatively coupled to the computing system and configured to provide data to the computing system to allow for three-dimensional tracking of the HIFU treatment transducer array relative to the patient. The one or more sensors may be selected from the group consisting of: a joint position sensor, an image capture device, an electromagnetic tracking sensor, a LIDAR device, an IMU, and an elongation sensor. The method further may comprise providing one or more sensors operatively coupled to the computing system and configured to provide data to the computing system to allow for three-dimensional tracking of the patient. The one or more sensors may be selected from the group consisting of: a joint position sensor, an image capture device, an electromagnetic tracking sensor, a LIDAR device, an IMU, and an elongation sensor.

Another embodiment is directed to a method for positioning instrumentation for a minimally invasive intervention on a patient, comprising: providing an elongate guiding member having a proximal end, a distal end, and a guiding lumen defined therethrough, the distal end being configured to be positioned adjacent to a targeted intervention location within the patient, and providing an imaging transducer configured to be interfaced against the patient, the imaging transducer defining an imaging field of view which may be displayed upon an operatively coupled display device; wherein the imaging transducer is movably coupled to the elongate guiding member such that the field of view of the imaging transducer may be repositioned as the elongate guiding member is repositioned relative to the patient, such that the distal end of the elongate guiding member may be maintained within the field of view of the imaging transducer. The imaging transducer may be rotatably coupled to the elongate guiding member. The rotatable coupling may comprise a drive motor configured to produce oscillatory motion of at least a portion of the imaging transducer such that the field of view of the imaging transducer is swept in a pattern selected to capture the distal end of the elongate guiding member along with aspects of the patient adjacent the distal end of the elongate guiding member. The elongate guiding member may be an instrument selected from the group consisting of: a cannula, a needle, and a catheter. The elongate guiding member may be a needle configured to aspirate portions of tissue which may have been previously lysed in the targeted intervention location. The method further may comprise providing a HIFU treatment transducer array operatively coupled to a computing system, wherein the computing system is configured to position a treatment focus of the HIFU treatment transducer array in alignment to treat at least a portion of the targeted intervention location of the patient, and to operate the HIFU treatment transducer array to controllably create a pulsatile wavefront of ultrasound radiation directed at the treatment focus, the pulsatile wavefront configured to produce one or more vapor bubbles within the targeted intervention location and to controllably produce cavitation of the one of more vapor bubbles such that a controllably lysed portion of the tissue of the patient at the targeted intervention location is created. The method further may comprise providing a delivery interface positioned between the HIFU treatment transducer array and the patient and configured to provide an efficient medium for conducting sound energy between the HIFU treatment transducer array and the patient. The method further may comprise providing a layer of acoustic gel interposed between the delivery interface and the patient and configured to further assist in efficient transmission between the HIFU treatment transducer array and the patient. The treatment focus may have a maximum dimension of about 5 millimeters. The treatment focus may have a maximum dimension of about 100 microns. The HIFU treatment transducer array may have an output frequency of between about 1 MHz and about 3 MHz. The HIFU treatment transducer array may have an output power of between about 300 watts and about 4,000 watts. The pulsatile wavefront may comprise a plurality of waves formed into a pulse, the pulse having a pulse duration of between about 1 and about 30 milliseconds. The computing system may be configured to produce the pulsatile wavefront for the pulse duration, followed by a pause of between about 0.1 second to about 1 second, before initiating another pulse. The waves may be configured to have a pressure amplitude received at the treatment focus of greater than about 60 MPa. The waves may be configured to have a negative peak pressure received at the treatment focus of between about 10 MPa and about 15 MPa. The controllably lysed portion may be created, at least in part, by an acoustic fountain reaction associated with the cavitation of the one or more vapor bubbles.

Another embodiment is directed to a minimally invasive method for treating a targeted tissue structure of a patient, comprising: providing an electromechanical support assembly having a proximal portion and a distal portion; providing a computing system operatively coupled to the electromechanical support assembly; providing a HIFU treatment transducer array coupled to the distal portion of the electromechanical support assembly and operatively coupled to the computing system; and providing an elongate guiding member movably coupled to the HIFU treatment transducer array and having a proximal end, a distal end, and a guiding lumen defined therethrough, the distal end being configured to be positioned adjacent to the targeted tissue structure within the patient; and utilizing the computing system to operate the electromechanical support assembly to control a position of the transducer assembly relative to the patient such that a treatment focus of the HIFU treatment transducer array is aligned to treat at least a portion of the targeted tissue structure of the patient, and to operate the HIFU treatment transducer array to controllably create a pulsatile wavefront of ultrasound radiation directed at the treatment focus, the pulsatile wavefront configured to produce one or more vapor bubbles within the targeted tissue structure and to controllably produce cavitation of the one of more vapor bubbles such that a controllably lysed portion of the targeted tissue structure is created; and wherein the elongate guiding member is configured to be utilized to remove the controllably lysed portion. The elongate guiding member may be movably coupled relative to the treatment focus of the HIFU treatment transducer array such that the distal portion of the elongate guiding member may be inserted along a predetermined axis selected to be aligned with the position of the treatment focus and controllably lysed portion. The elongate guiding member may be an instrument selected from the group consisting of: a cannula, a needle, and a catheter. The elongate guiding member may be a needle configured to aspirate the controllably lysed portion. The electromechanical support assembly may comprise a plurality of elongate portions coupled by one or more movable joints. The one or more movable joints may be coupled to one or more encoders operatively coupled to the computing system and configured to provide inputs to the computing system for determining positions of the one or more movable joints. The electromechanical support assembly may comprise one or more sensors configured to sense one or more loads within the electromechanical support assembly associated with a physical interface between the HIFU treatment transducer array and the patient. The one or more sensors may be chosen from the group consisting of: a joint load sensor, a joint torque sensor, a strain gauge, and a deflection gauge. The method further may comprise providing one or more motors operatively coupled to the electromechanical support assembly and configured to apply loads thereto to maintain or change position or orientation of the electromechanical support assembly. The electromechanical support assembly may comprise a robotic arm. The computing system may be further configured to operate the electromechanical support assembly to control an orientation of the transducer assembly relative to the patient. The electromechanical support assembly may be controlled by the computer in response to inputs provided by an operator. The inputs provided by the operator may be manual electromechanical support assembly movement commands. The inputs provided by the operator may be commands for the electromechanical support assembly to follow a prescribed set of movements. The electromechanical support assembly may be controlled by the computer automatically in response to prescribed inputs provided by an operator. The HIFU treatment transducer array may be operatively coupled to the computing system using a wireless connectivity configuration. The HIFU treatment transducer array may be operatively coupled to the computing system using a wired connectivity configuration. The electromechanical support assembly may be operatively coupled to the computing system using a wireless connectivity configuration. The electromechanical support assembly may be operatively coupled to the computing system using a wired connectivity configuration. The method further may comprise providing an imaging ultrasound transducer having a ultrasound imaging field of view aligned to capture at least a portion of the treatment focus of the HIFU treatment transducer array. The HIFU treatment transducer array and the imaging ultrasound transducer may be both coupled to the distal portion of the electromechanical support assembly. The method further may comprise providing a delivery interface positioned between the HIFU treatment transducer array and the patient and configured to provide an efficient medium for conducting sound energy between the HIFU treatment transducer array and the patient. The method further may comprise providing a layer of acoustic gel interposed between the delivery interface and the patient and configured to further assist in efficient transmission between the HIFU treatment transducer array and the patient. The treatment focus may have a maximum dimension of about 5 millimeters. The treatment focus may have a maximum dimension of about 100 microns. The HIFU treatment transducer array may have an output frequency of between about 1 MHz and about 3 MHz. The HIFU treatment transducer array may have an output power of between about 300 watts and about 4,000 watts. The pulsatile wavefront may comprise a plurality of waves formed into a pulse, the pulse having a pulse duration of between about 1 and about 30 milliseconds. The computing system may be configured to produce the pulsatile wavefront for the pulse duration, followed by a pause of between about 0.1 second to about 1 second, before initiating another pulse. The waves may be configured to have a pressure amplitude received at the treatment focus of greater than about 60 MPa. The waves may be configured to have a negative peak pressure received at the treatment focus of between about 10 MPa and about 15 MPa. The controllably lysed portion may be created, at least in part, by an acoustic fountain reaction associated with the cavitation of the one or more vapor bubbles.

Another embodiment is directed to a minimally invasive method for treating a targeted tissue structure of a patient, comprising: providing an electromechanical support assembly having a proximal portion and a distal portion; providing a computing system operatively coupled to the electromechanical support assembly; providing a HIFU treatment transducer array coupled to the distal portion of the electromechanical support assembly and operatively coupled to the computing system; and utilizing the computing system to operate the electromechanical support assembly to control a position of the transducer assembly relative to the patient such that an interfacial load between the transducer assembly and the patient is controlled, and such that a treatment focus of the HIFU treatment transducer array is aligned to treat at least a portion of the targeted tissue structure of the patient, and utilizing the computing system to operate the HIFU treatment transducer array to controllably create a pulsatile wavefront of ultrasound radiation directed at the treatment focus, the pulsatile wavefront configured to produce one or more vapor bubbles within the targeted tissue structure and to controllably produce cavitation of the one of more vapor bubbles such that a controllably lysed portion of the targeted tissue structure is created. The electromechanical support assembly may comprise a plurality of elongate portions coupled by one or more movable joints. The one or more movable joints may be coupled to one or more encoders operatively coupled to the computing system and configured to provide inputs to the computing system for determining positions of the one or more movable joints. The electromechanical support assembly may comprise one or more sensors configured to sense one or more loads within the electromechanical support assembly associated with the interfacial load between the HIFU treatment transducer array and the patient. The computing system may be configured to maintain the interfacial load below a predetermined maximum. The computing system may be configured to maintain the interfacial load above a predetermined minimum and below a predetermined maximum during a period of treatment of the patient. The computing system may be configured to maintain a relative orientation between the HIFU treatment transducer and a most immediately adjacent portion of the patient's body while also maintaining the interfacial load above a predetermined minimum and below a predetermined maximum during a period of treatment of the patient. The computing system may be configured to facilitate a repositioning of the HIFU treatment transducer relative to the patient while also maintaining the relative orientation between the HIFU treatment transducer and most immediately adjacent portion of the patient's body, as well as also maintaining the interfacial load above a predetermined minimum and below a predetermined maximum during a period of treatment of the patient. The one or more sensors may be chosen from the group consisting of: a joint load sensor, a joint torque sensor, a strain gauge, and a deflection gauge. The method further may comprise providing one or more motors operatively coupled to the electromechanical support assembly and configured to apply loads thereto to maintain or change position or orientation of the electromechanical support assembly. The electromechanical support assembly may comprise a robotic arm. The computing system may be further configured to operate the electromechanical support assembly to control an orientation of the transducer assembly relative to the patient. The electromechanical support assembly may be controlled by the computer in response to inputs provided by an operator. The inputs provided by the operator may be manual electromechanical support assembly movement commands. The inputs provided by the operator may be commands for the electromechanical support assembly to follow a prescribed set of movements. The electromechanical support assembly may be controlled by the computer automatically in response to prescribed inputs provided by an operator. The HIFU treatment transducer array may be operatively coupled to the computing system using a wireless connectivity configuration. The HIFU treatment transducer array may be operatively coupled to the computing system using a wired connectivity configuration. The electromechanical support assembly may be operatively coupled to the computing system using a wireless connectivity configuration. The electromechanical support assembly may be operatively coupled to the computing system using a wired connectivity configuration. The method further may comprise providing an imaging ultrasound transducer having a ultrasound imaging field of view aligned to capture at least a portion of the treatment focus of the HIFU treatment transducer array. The HIFU treatment transducer array and the imaging ultrasound transducer may be both coupled to the distal portion of the electromechanical support assembly. The method further may comprise providing a delivery interface positioned between the HIFU treatment transducer array and the patient and configured to provide an efficient medium for conducting sound energy between the HIFU treatment transducer array and the patient. The method further may comprise providing a layer of acoustic gel interposed between the delivery interface and the patient and configured to further assist in efficient transmission between the HIFU treatment transducer array and the patient. The treatment focus may have a maximum dimension of about 5 millimeters. The treatment focus may have a maximum dimension of about 100 microns. The HIFU treatment transducer array may have an output frequency of between about 1 MHz and about 3 MHz. The HIFU treatment transducer array may have an output power of between about 300 watts and about 4,000 watts. The pulsatile wavefront may comprise a plurality of waves formed into a pulse, the pulse having a pulse duration of between about 1 and about 30 milliseconds. The computing system may be configured to produce the pulsatile wavefront for the pulse duration, followed by a pause of between about 0.1 second to about 1 second, before initiating another pulse. The waves may be configured to have a pressure amplitude received at the treatment focus of greater than about 60 MPa. The waves may be configured to have a negative peak pressure received at the treatment focus of between about 10 MPa and about 15 MPa. The controllably lysed portion may be created, at least in part, by an acoustic fountain reaction associated with the cavitation of the one or more vapor bubbles.

Another embodiment is directed to a robotic medical intervention method for treating a targeted tissue structure of a patient, comprising: providing a robotic system base; a computing system operatively coupled to the robotic system base; a plurality of robotic arms, each having a proximal and a distal end, the proximal ends being movably coupled to the robotic system base; and a plurality of interventional end effectors, each interventional end effector coupled to the distal end of one of the plurality of robotic arm distal ends; wherein at least one of the intervention end effectors comprises a HIFU treatment transducer array; and utilizing the computing system to operate one of the plurality of robotic arms to control a position of the transducer assembly relative to the patient such that a treatment focus of the HIFU treatment transducer array is aligned to treat at least a portion of the targeted tissue structure of the patient, and to operate the HIFU treatment transducer array to controllably create a pulsatile wavefront of ultrasound radiation directed at the treatment focus, the pulsatile wavefront configured to produce one or more vapor bubbles within the targeted tissue structure and to controllably produce cavitation of the one of more vapor bubbles such that a controllably lysed portion of the targeted tissue structure is created. The computing system further may be configured to operate one of the plurality of robotic arms to control the position of the transducer assembly relative to the patient such that an interfacial load between the transducer assembly and the patient is controlled. At least one of the plurality or robotic arms may comprise a plurality of elongate portions coupled by one or more movable joints. The one or more movable joints may be coupled to one or more encoders operatively coupled to the computing system and configured to provide inputs to the computing system for determining positions of the one or more movable joints. The plurality of robotic arms may comprise one or more sensors configured to sense one or more loads associated with the interfacial load between the HIFU treatment transducer array and the patient. The computing system may be configured to maintain the interfacial load below a predetermined maximum. The computing system may be configured to maintain the interfacial load above a predetermined minimum and below a predetermined maximum during a period of treatment of the patient. The computing system may be configured to maintain a relative orientation between the HIFU treatment transducer and a most immediately adjacent portion of the patient's body while also maintaining the interfacial load above a predetermined minimum and below a predetermined maximum during a period of treatment of the patient. The computing system may be configured to facilitate a repositioning of the HIFU treatment transducer relative to the patient while also maintaining the relative orientation between the HIFU treatment transducer and most immediately adjacent portion of the patient's body, as well as also maintaining the interfacial load above a predetermined minimum and below a predetermined maximum during a period of treatment of the patient. The one or more sensors are chosen from the group consisting of: a joint load sensor, a joint torque sensor, a strain gauge, and a deflection gauge. The computing system may be further configured to operate at least one of the plurality of robotic arms to control an orientation of the transducer assembly relative to the patient. The position of the HIFU treatment transducer may be controlled by the computer in response to inputs provided by an operator. The inputs provided by the operator may be manual HIFU treatment transducer movement commands. The inputs provided by the operator may be commands for the HIFU treatment transducer to follow a prescribed set of movements. The position of the HIFU treatment transducer may be controlled by the computer automatically in response to prescribed inputs provided by an operator. The HIFU treatment transducer array may be operatively coupled to the computing system using a wireless connectivity configuration. The HIFU treatment transducer array may be operatively coupled to the computing system using a wired connectivity configuration. The plurality of robotic arms may be operatively coupled to the computing system using a wireless connectivity configuration. The plurality of robotic arms may be operatively coupled to the computing system using a wired connectivity configuration. The method further may comprise providing an imaging ultrasound transducer having a ultrasound imaging field of view aligned to capture at least a portion of the treatment focus of the HIFU treatment transducer array. The HIFU treatment transducer array and the imaging ultrasound transducer may be both coupled to the distal portion of at least one of the robotic arms. The method further may comprise providing a delivery interface positioned between the HIFU treatment transducer array and the patient and configured to provide an efficient medium for conducting sound energy between the HIFU treatment transducer array and the patient. The method further may comprise providing a layer of acoustic gel interposed between the delivery interface and the patient and configured to further assist in efficient transmission between the HIFU treatment transducer array and the patient. The treatment focus may have a maximum dimension of about 5 millimeters. The treatment focus may have a maximum dimension of about 100 microns. The HIFU treatment transducer array may have an output frequency of between about 1 MHz and about 3 MHz. The HIFU treatment transducer array may have an output power of between about 300 watts and about 4,000 watts. The pulsatile wavefront may comprise a plurality of waves formed into a pulse, the pulse having a pulse duration of between about 1 and about 30 milliseconds. The computing system may be configured to produce the pulsatile wavefront for the pulse duration, followed by a pause of between about 0.1 second to about 1 second, before initiating another pulse. The waves may be configured to have a pressure amplitude received at the treatment focus of greater than about 60 MPa. The waves may be configured to have a negative peak pressure received at the treatment focus of between about 10 MPa and about 15 MPa. The controllably lysed portion may be created, at least in part, by an acoustic fountain reaction associated with the cavitation of the one or more vapor bubbles.

### DETAILED DESCRIPTION

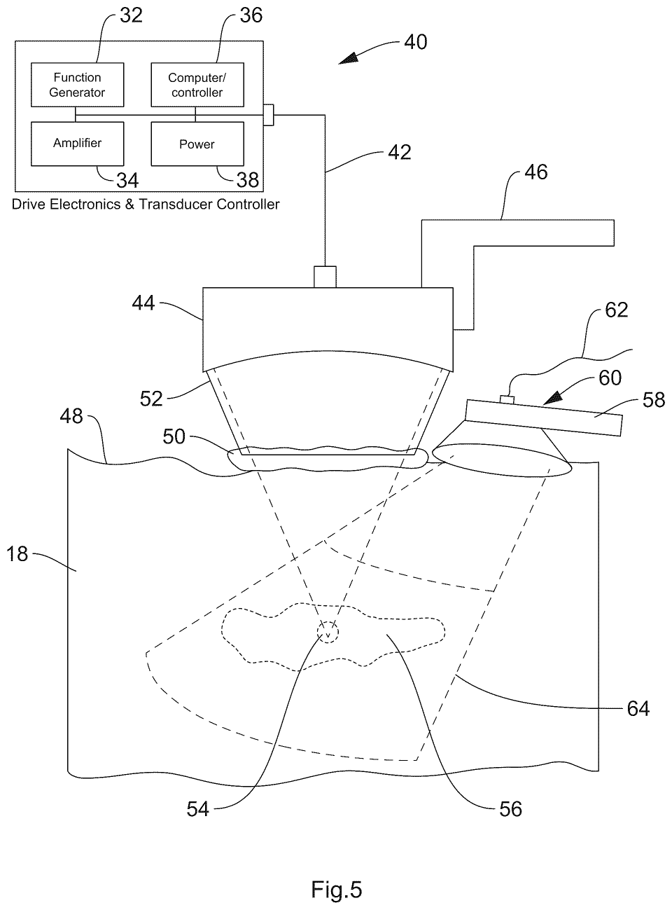

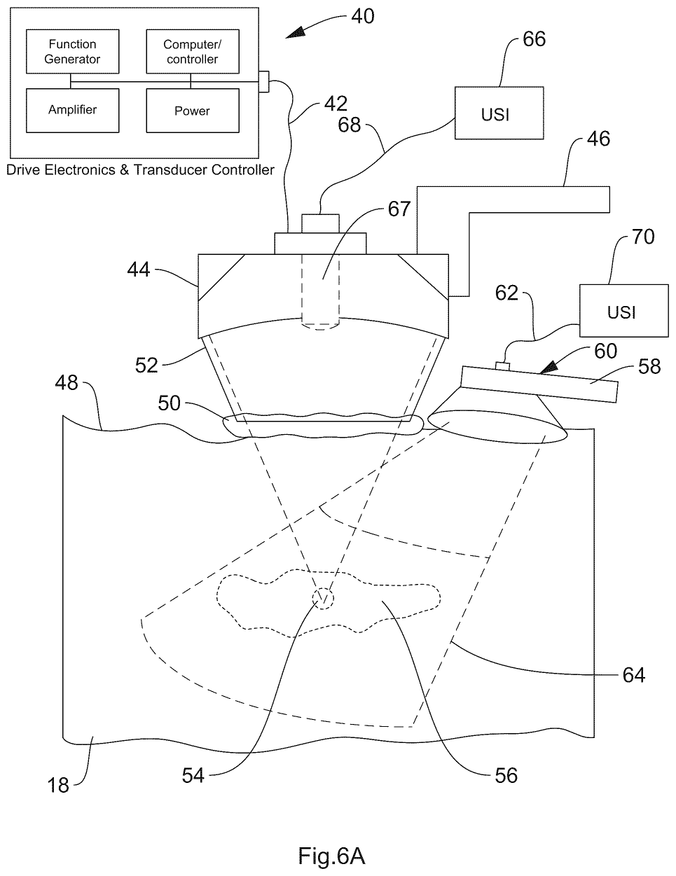

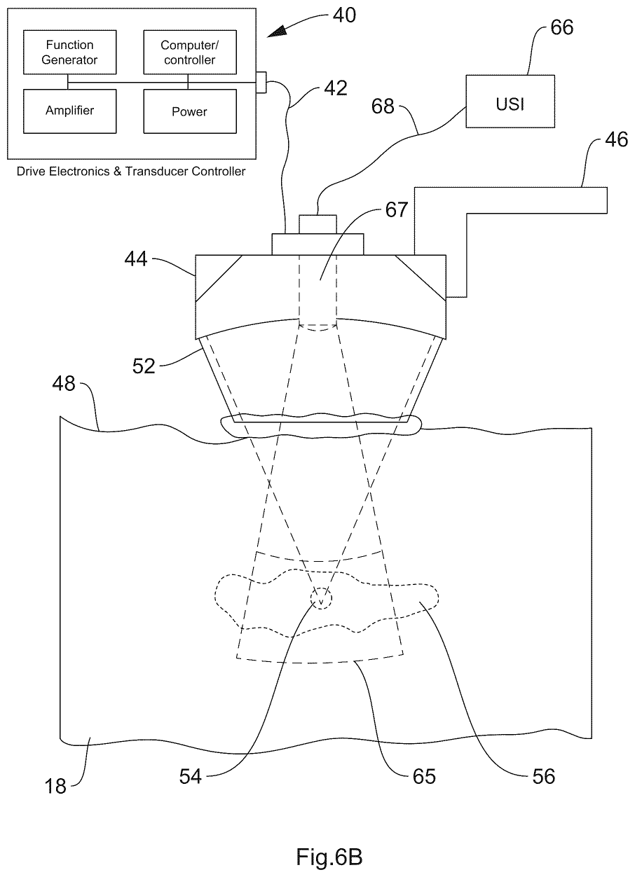

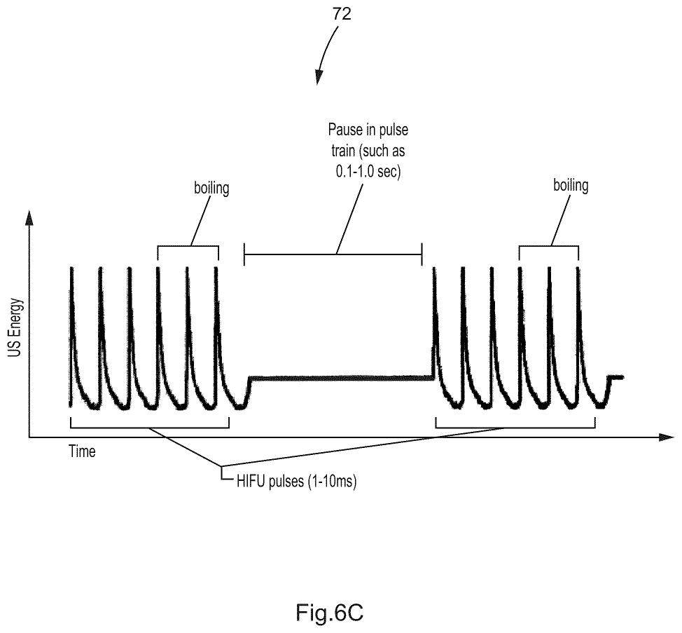

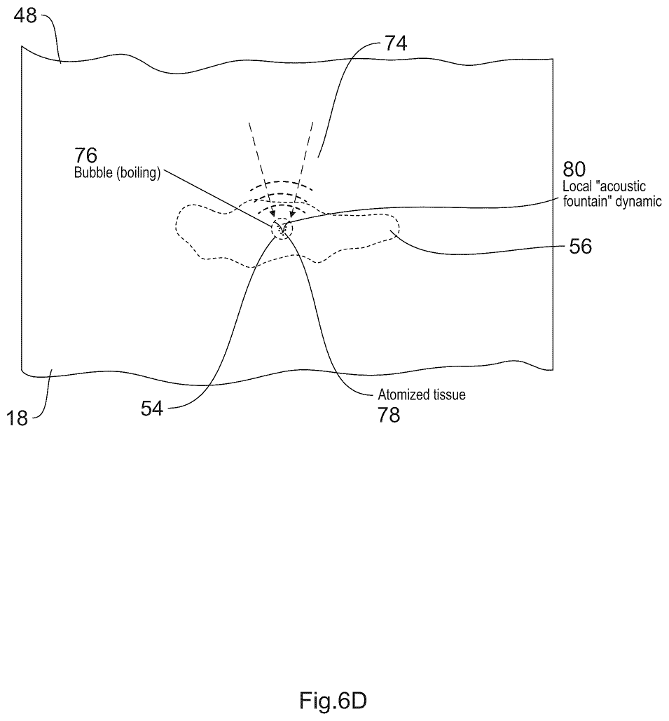

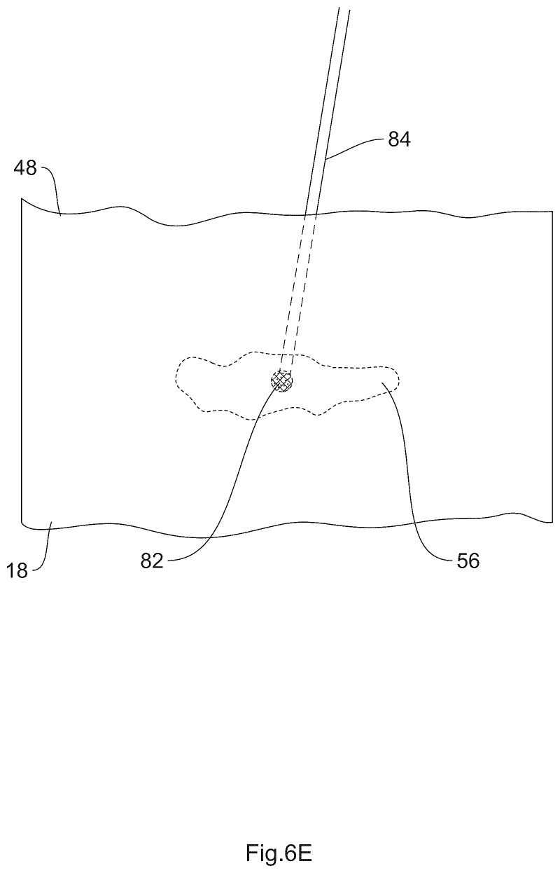

Recent advancements in transducer technology and configuration have brought about a group of evolving technologies which relate to the use of high intensity focused ultrasound, or “HIFU”, in various clinical scenarios. While attractive for minimally invasive interventional scenarios wherein attempts may be made to minimize access by conventional transcutaneous surgical wound, delivery of energy to one or more targeted tissue structures in conventional HIFU configurations has been associated with potentially undesirable elevations in temperature at sites local to the focus of the pertinent HIFU transducer assembly, as well as other side effects, and as a result, certain indications do not appear well suited for conventional HIFU intervention. More recently, boiling histotripsy (“BH”) HIFU techniques and configurations have been developed, which may be utilized to avoid certain side effects of conventional HIFU. Various aspects of BH are described, for example, in U.S. Pat. Nos. 8,876,740, 9,700,742, and 9,498,651, each of which is incorporated by reference herein in its entirety. In various embodiments, so-called boiling histotripsy configurations have been developed wherein a relatively low pressure wavefront is directed at one or more nucleated vapor bubbles, causing controlled cavitation and resultant controlled lysis of cells and/or tissue within a treatment focus volume (54). Referring ahead to FIGS. 6C and 6D, for example, a series of pulses (72) from BH transducer assemblies such as those described in the aforementioned incorporated references may be utilized to direct (74) ultrasound energy across the skin (48) toward a targeted tissue structure (56) and create very focused and controlled atomization, emulsification, and/or destruction of tissue through the use of bubbles/boiling (76) and associated cavitation thereof, at a relatively discrete focal point or volume (54), which may lead to what has been described as a local “acoustic fountain” (80) type of reaction configuration at the focal point or volume (54). Referring to FIG. 6E, an elongate instrument (84) such as an aspiration needle may be utilized to remove the locally atomized, emulsified, and/or destroyed tissue portions (82) when desired, preferably with the use of image guidance from modalities such as radiography, fluoroscopy, and/or imaging ultrasound to assist in locating such elongate instrument (84) distal portion at the location of the focal point or volume (54) within the targeted tissue structure (56). In various embodiments, a computing system may be operatively coupled to a HIFU treatment transducer or transducer array (such as, for example, element 67 of assembly 44) and configured to operate such transducer array to controllably create a pulsatile wavefront of ultrasound radiation directed at the chosen treatment focus (54). The pulsatile wavefront may be configured to produce or nucleate one or more vapor bubbles by heating up the tissue in the area of the treatment focus (54) to about 100 degrees celsius within a few milliseconds. Continued energy from wavefronts within pulses, such as those shown in FIG. 6C, may be configured to controllably produce cavitation of the one or more vapor bubbles such that a controllably lysed portion of the targeted tissue structure is created, and which may be removed, such as by aspiration. Referring again to FIG. 6C, each pulse may be in the range of 1-10 milliseconds, or as long as 1-30 milliseconds in some embodiments; then a pause in the pulse train (such as between about 0.1 seconds and 1 second; thereby resulting in a “duty cycle” of approximately 2% or less) may be executed by the controller or computer, followed by an other pulse of wavefronts, until desired cavitation has occurred at the treatment focus (54). In various embodiments, the treatment focus (54) may have a maximum dimension of about 5 millimeters; in other embodiments the treatment focus (54) may be configured to be as small as 100 microns. In various embodiments, the HIFU treatment transducer array may have an output frequency of between about 1 MHz and about 3 MHz. In various embodiments, the HIFU treatment transducer array may have an output power of between about 300 watts and about 4,000 watts. Suitable transducers may comprise, for example, piezoelectric materials selected to oscillate to create waves and wavefronts with desired characteristics. The waves with in each wavefront of a pulse may be configured to have a pressure amplitude as received at the treatment focus (54) of greater than about 60 MPa, but importantly may have a negative peak pressure in the relatively low range of between about 10 MPa and about 15 MPa. Referring to FIGS. 5, 6A, and 6B, an a function generator (32), amplifier (34), computer, computing system, or controller device (36), and power source (38) may be operatively coupled (42) to a HIFU transducer array (44) and configured to deliver, through the use of a delivery interface (52) which preferably comprises an efficient medium for conducting sound energy (such as water which may be de-ionized and/or de-gassed) between the transducer array and subject tissue interface, and a layer of acoustic gel (50) to assist in transmission efficiency, a pulsatile BH HIFU configuration such as that shown in FIG. 6C or described in the aforementioned incorporated references, to a discrete focus point or volume (54) within a targeted tissue structure (56). The system configurations of FIGS. 5, 6A, and 6B illustrate that a computing system may be operatively coupled, such as via wired or wireless interface (such as via IEEE 802.11 wireless connectivity or mobile wireless connectivity, for example) to the various components, such as to the electromechanical support assembly configurations (such as element 146), the interventional and imaging ultrasound transducers and related components (such as elements 44, 60, 67, 66, 70) to control and monitor such components; in other embodiments featuring other intercoupled electronic components such as sensors (such as IMUs, optical tracking sensors, joint encoders, image capture devices, electromagnetic tracking sensors, LIDAR sensors, and strain or elongation sensors, all of which are discussed in further detail below), storage devices (such as to make certain preoperative or intraoperative information available, as described herein), such components may be similarly operatively coupled to the computing system. Further, the computing system may be programmable and/or controllable by inputs, predetermined variables, predetermined paths. In scenarios wherein a pocket of gas or air is positioned in the pathway between the transducer array (44) and the targeted tissue structure, additional efficient medium material (again, such as water which may be de-ionized and/or de-gassed) may be injected or placed in such pathway to improve transmission efficiency between the transducer array (44) and targeted tissue structure. In a relatively basic embodiment, such as illustrated in FIG. 5, the HIFU transducer array (44) may be held in place by a movable mounting structure (46), and guidance may be assisted via the use of conventional ultrasound imaging, such as via systems such as those available under the tradename Sequoia® from Siemens, which may incorporate an ultrasound imaging head (60) containing one or more ultrasound transducers, which are operatively coupled to an ultrasonic imaging controller (70), such as a computer system; the ultrasound imaging head (60) may be configured to be held in place by a movable mounting structure (58), and may be configured to provide ultrasound image data pertaining to one or more “slices” that pertain to a pertinent field of view (64) of tissue scanned by the ultrasound imaging head (70).

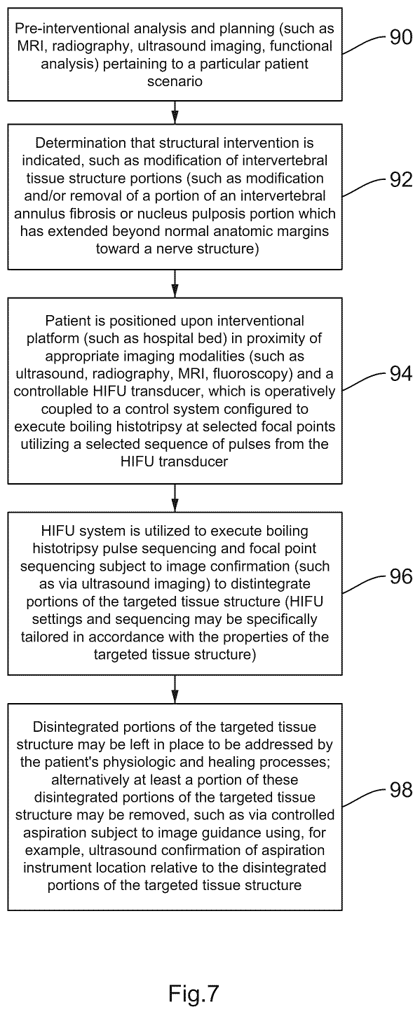

Referring to FIG. 6B, another embodiment is illustrated with an ultrasound system (66) operatively coupled (68) to an imaging ultrasound transducer (67) that is coupled to the HIFU transducer array (44) and configured to provide a field of view (65) which is at least somewhat pre-aligned with the treatment focus (54) of the HIFU transducer array (44). FIG. 6A illustrates an embodiment featuring both imaging ultrasound integrated into the transducer array (44) structure, but also separate ultrasound imaging (70, 60, 62) for additional image-based confirmation of interventional activity at the targeted tissue structure (56). Referring to FIG. 7, a method and configuration are illustrated wherein aspects of the aforementioned system configurations may be utilized. A patient may undergo pre-interventional analysis and planning (such as MRI, radiography, ultrasound imaging, functional analysis) pertaining to a particular patient scenario (90), such as herniation of an intervertebral disc of the spine of the patient. With determination (92) that structural intervention is indicated, such as modification of intervertebral tissue structure portions (such as modification and/or removal of a portion of an intervertebral annulus fibrosis or nucleus pulposis portion which has extended beyond normal anatomic margins toward a nerve structure), a medical team may prepare for intervention. The patient may be positioned upon an interventional platform (such as hospital bed) in proximity of appropriate imaging modalities (such as ultrasound, radiography, MRI, fluoroscopy) and a controllable HIFU transducer, which may be operatively coupled to a control system configured to execute boiling histotripsy at selected focal points utilizing a selected sequence of pulses from the HIFU transducer (94). The HIFU system may be utilized to execute boiling histotripsy pulse sequencing and focal point sequencing subject to image confirmation (such as via ultrasound imaging) to distintegrate portions of the targeted tissue structure (HIFU settings and sequencing may be specifically tailored in accordance with the properties of the targeted tissue structure) (96). Disintegrated portions of the targeted tissue structure may be left in place to be addressed by the patient's physiologic and healing processes; alternatively at least a portion of these disintegrated portions of the targeted tissue structure may be removed, such as via controlled aspiration subject to image guidance using, for example, ultrasound confirmation of aspiration instrument location relative to the disintegrated portions of the targeted tissue structure (98).

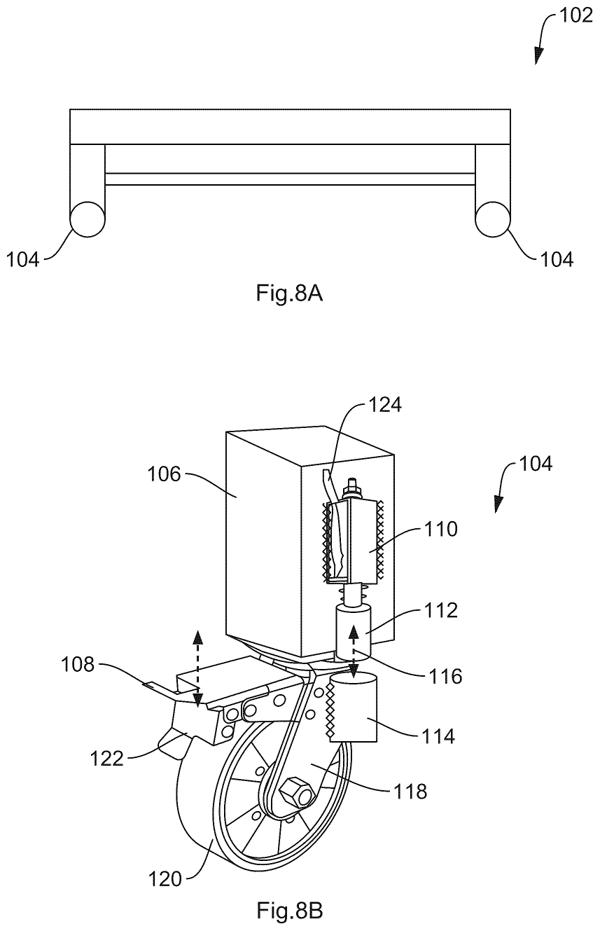

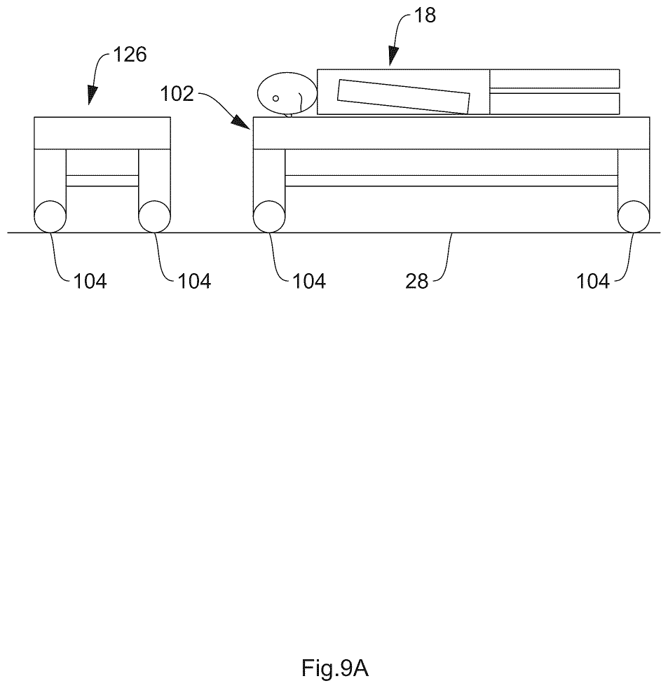

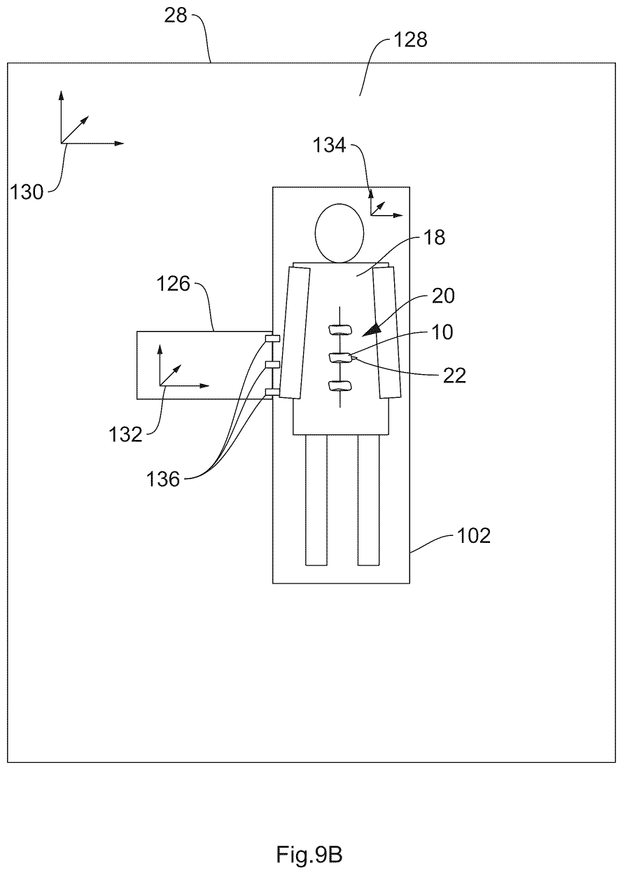

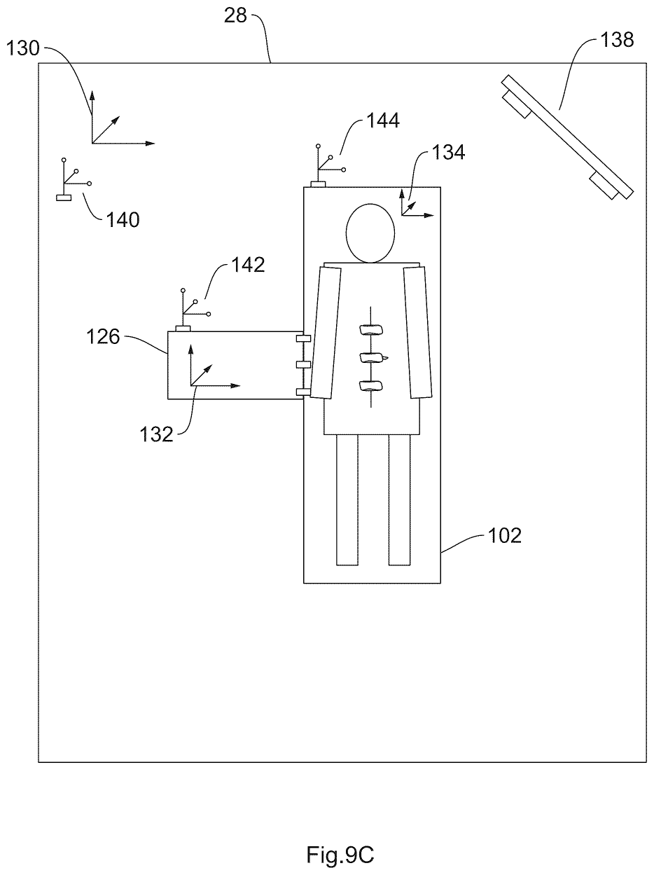

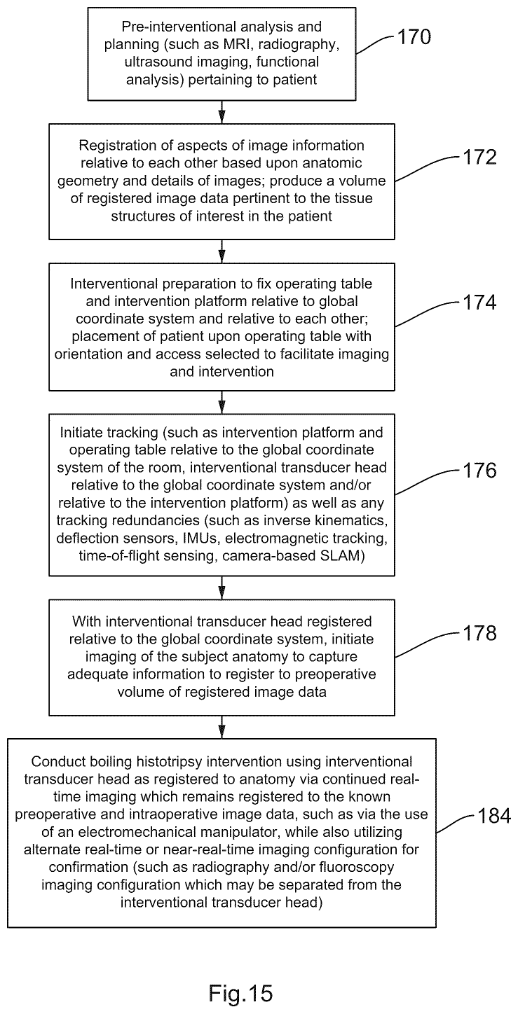

Referring to FIGS. 8A-28, additional aspects of other embodiments are illustrated in reference to a minimally invasive spine herniation intervention. For example, in various embodiments it may be desirable to utilize an electromechanical system, such as an electromechanical or robotic manipulator or arm, to assist in locating a HIFU transducer head (44) relative to a patient (18) and targeted tissue structure. To assist with precision relative positioning and orientation of a manipulator relative to a patient, image guidance may be utilized, as well as a general stabilization of certain associated structures relative to each other (for example, it may be helpful to generally prevent the hospital bed holding the patient from moving around on the operating room floor during the procedure). Referring to FIGS. 8A and 8B, a hospital or surgical bed (102) may be configured to have controllably braked wheel assemblies (104) which may be configured to not only have conventional braking (such as through operator-foot-based depression of a braking interface member 108 to engage a brake member 122 against the wheel 120) to prevent further rolling motion of the wheels (120), but also braking and/or temporary fixation at the roll rotation axis between the leg portion (106) of the bed (102) and the lower wheel frame (118) portion (such as via a remotely actuated solenoid 110 configured to urge a fixation shaft 112 into (and back out of upon release command) a fixation socket 114 that is fixedly coupled to the lower wheel frame 118). In one embodiment, for example, through a push of a button, the roll axes of the four wheel assemblies (104) of a surgical bed may become controllably fixed in that roll axis. Similarly, the depicted manual wheel brakes (122) may be configured to be electromechanically actuated, such as via solenoids. Thus referring to FIG. 9A, a patient (18) upon a surgical bed (102) may be positioned adjacent an interventional cart (126), and the two may be locked into place relative to each other, and relative to the operating room (28). FIG. 9B illustrates a view from the ceiling of the operating room (28) down toward the patient (18) to show the bed (102) and interventional cart (126) braked in position against each other; the embodiment of FIG. 9B also illustrates some removable coupling locks (136) configured to detachably latch the two platforms (126, 102) relative to each other for added stability. FIG. 9B illustrates a portion of the patient's (18) spine (20) which may underly his or her closed (i.e., without conventional surgical wound approach) skin, with an intervertebral disc (10) herniation (22) shown as a target for a boiling histotripsy intervention. Coordinate systems of the operating room (130), interventional platform (132) and surgical bed (134) are illustrated as a reminder that in certain interventional configurations, it may be critical to maintain an understanding of the positions and orientations of these coordinate systems (130, 132, 134) relative to each other. Referring to FIG. 9C, optical tracking fiducials (140, 142, 144) may be coupled to the operating room (130), interventional platform (132), and surgical bed (134), as shown, to assist in tracking any position and/or orientation changes of these structures relative to each other, such as via a precision multi-camera-based optical tracking system (138), such as those available from Northern Digital, Inc.

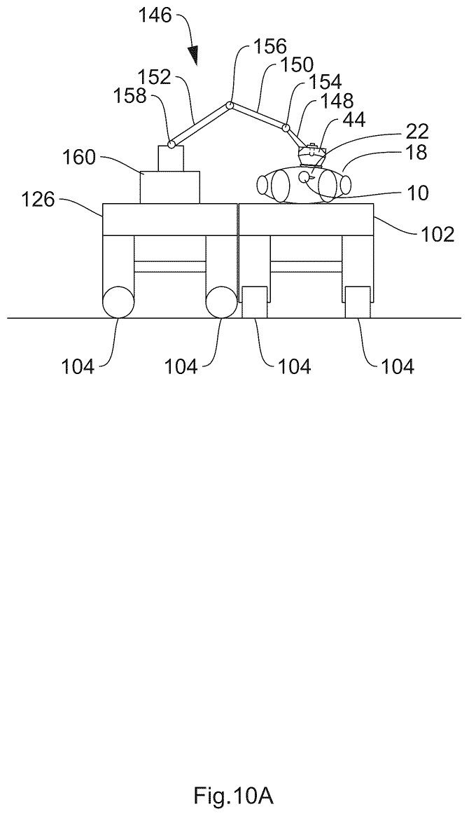

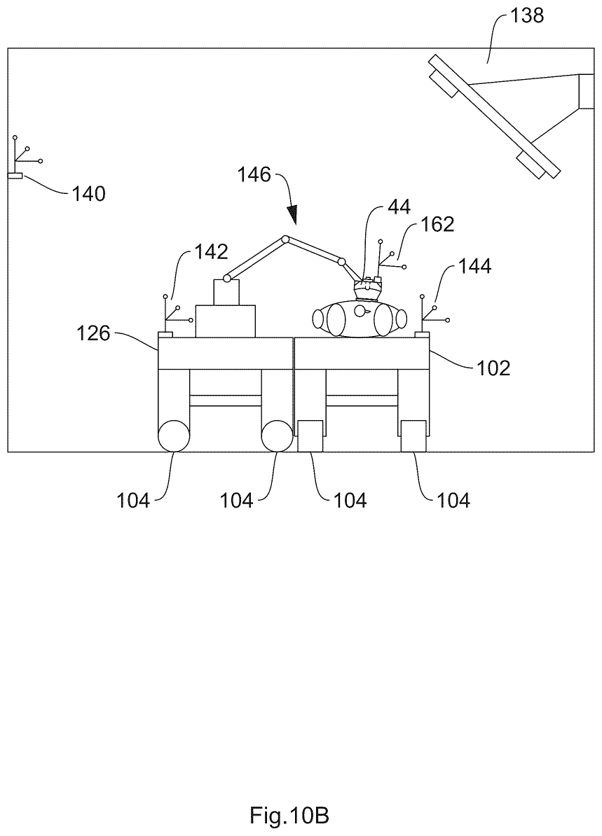

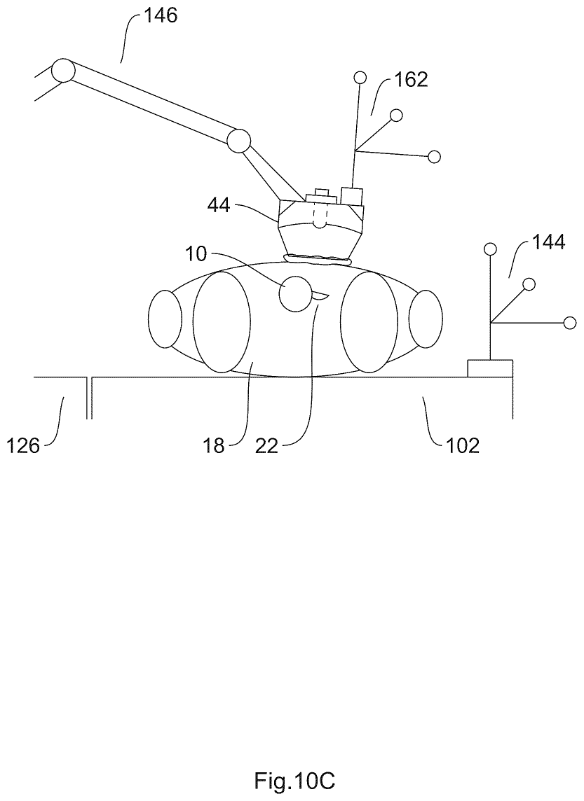

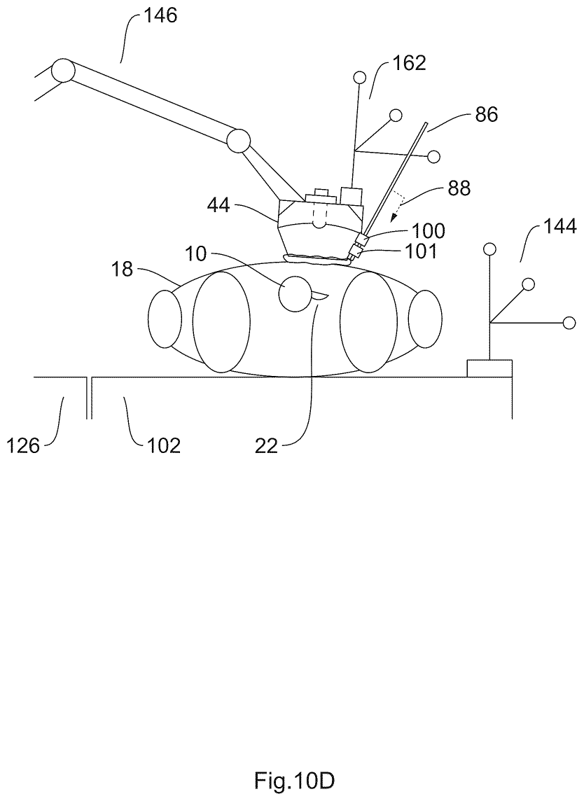

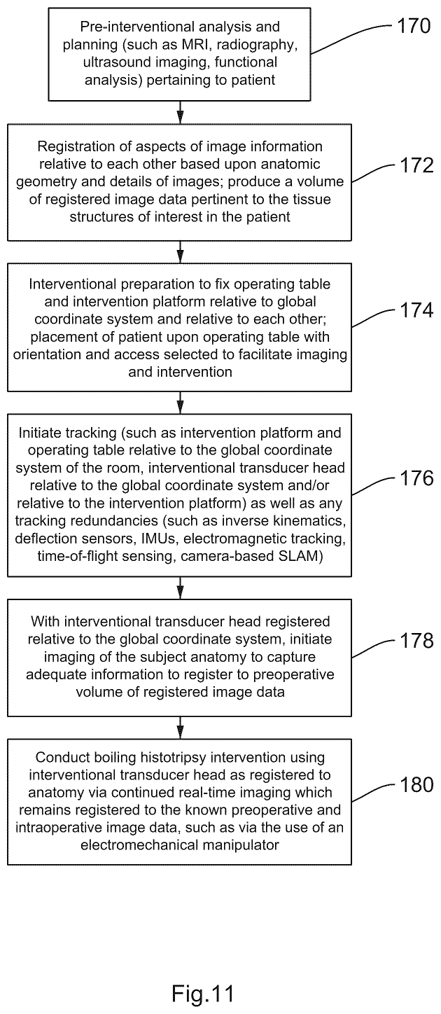

Referring to FIGS. 10A-10D, an electromechanical manipulator or electromechanical support assembly (146), such as a robotic arm (such as those available, for example, from manufacturers such as Barrett Technology, Inc. of Newton, Massachusetts, or Kuka A. G. of Augsburg, Germany) may comprise various elongate segments, motors or actuators, and joints (which may be operatively coupled to joint encoders to facilitate determination of joint angles or positions, for example) and may be utilized to precisely reposition and reorient a BH HIFU transducer head (44) relative to the anatomy of the patient (18) for precision intervention. The manipulator assembly (146) may comprise a stabilizing base (160) which may be fixedly coupled to the interventional platform (126). A series of controllable joints (154, 156, 158) positioned in between substantially rigid elongate linkage structures (148, 150, 152, 160) may be utilized, as operatively coupled to a computing system, for example, to controllably position and orient the transducer head (44) relative to the patient (18), and such repositioning and reorientation may be conducted manually, as shown in FIG. 10A, but preferably is conducted with constantly updated determinations of position and orientation of the relevant associated structures, such as the manipulator (146), the transducer head (44), the platforms (102, 126), and the patient (18) anatomy. Initial calibration, kinematic relationships, and knowledge of joint positions of the manipulator (146) may be utilized to gain a basic understanding of the position and orientation of the manipulator (146). Referring to FIGS. 10B and 10C, one or more tracking fiducials (162) may also be coupled directly to the transducer head (44) or other structure associated with the manipulator (146) to assist in gaining further determination of the position and orientation of various structures before, during, and after providing energy for boiling histotripsy through the transducer head (44). Referring to FIG. 10D, a configuration similar to that of FIG. 10C is illustrated, with exception that an elongate interventional instrument (86), such as an aspiration needle, injection needle, or cannula, is movably coupled, such as by a set of small linear bearing clamps (100, 101), to the transducer head (44) such that the elongate instrument (86) may be advanced/retracted (88) along a predicted axis relative to the orientation of the transducer head, such as an axis that places a distal portion of the elongate instrument (86) at the position of the focus of the transducer head (44) at full insertion (88) of the elongate instrument. Thus referring to FIG. 11, a method and configuration are illustrated wherein aspects of the aforementioned system configurations may be utilized. The patient may undergo pre-interventional analysis and planning (such as MRI, CT, fluoroscopy, radiography, ultrasound imaging, functional analysis) pertaining to patient and subject targeted tissue structure (170). Resultant image information may be registered (i.e., such that the coordinate systems are positioned and oriented in anatomic alignment relative to each other, and relative to associated instrumentation pertinent to the procedure, in a global coordinate system) based upon anatomic geometry and details of images (may be accomplished, at least in part, by image processing computer configurations) such that image information from more than one source may be geometrically utilized together as a volume or grouping of registered image data that is pertinent to the tissue structures of interest in the patient (172). In various embodiments both preoperative and intraoperative image data may be registered to the coordinate system of the HIFU treatment transducer, to allow for image-based navigation of the transducer within the image data. Interventional preparation may be conducted to fix operating table and intervention platform relative to global coordinate system of the operating room, and relative to each other; the patient may be placed upon the operating table with orientation and access selected to facilitate the proposed intervention and associated imaging (174).

Tracking may be initiated (such as tracking of the intervention platform and operating table relative to the global coordinate system of the operating room, interventional transducer head relative to the global coordinate system and/or relative to the intervention platform) as well as any tracking redundancies (such as inverse kinematics through the manipulator, deflection sensors (such as conductive or optical strain or deflection gauges) which may be integrated into various components, inertial measurement units (“IMUs”, which may comprise accelerometers, gyros, and the like) which may be integrated into various components (for example an IMU may be coupled to each key segment of an electromechanical manipulator to assist in determining and confirming movements, accelerations, repositioning, reorientation), electromagnetic tracking (such as magnetic flux based position and/or orientation tracking sensors and systems, such as those available from Polhemus of Israel or Ascension Systems of Vermont), time-of-flight sensing (such as the systems and modules, including compact LIDAR systems, available from Hokuyo Automatic USA Corp of Indian Trail, North Carolina), camera or “computer vision” based tracking or pose determination techniques which may employ operatively coupled cameras such as those which may be featured as part of an optical tracking system (embodiments may include configurations referred to as simultaneous localization and mapping, or “SLAM”, configurations and techniques) (176).

Referring again to FIG. 11 and also to above-discussed configurations, with the interventional transducer head (44) registered relative to the global coordinate system, imaging of the subject anatomy may be initiated, such as by utilizing an ultrasound imaging transducer capability which may be integrated (such as shown in FIGS. 6A and 6B) into the interventional ultrasound head (44), to capture adequate information to register to preoperative volume of registered image data (178). Such a combination of intraoperative imaging and a registered dataset and interventional head (44) provides for an enhanced level of interventional control, as the system may be configured to assist the operator in aiming the BH HIFU focal point precisely at the targeted tissue structure of interest, based upon the updated relative location and orientation as determined by the registered system and updated information from all pertinent sensing configurations. Thus the interventional team may conduct a boiling histotripsy intervention using an interventional transducer head as registered to pertinent patient anatomy via continued real-time or near-real-time imaging which remains registered to the known preoperative and intraoperative image data, such as via the use of an electromechanical manipulator (180).

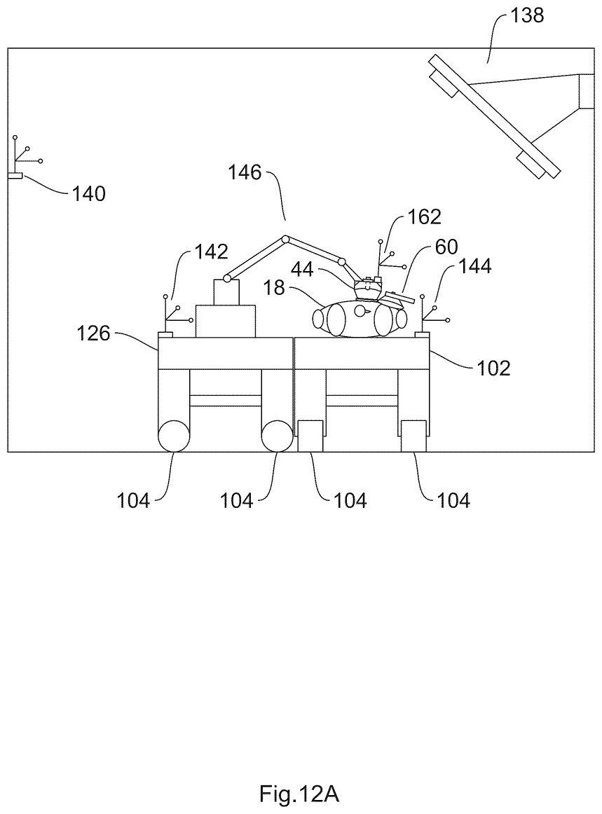

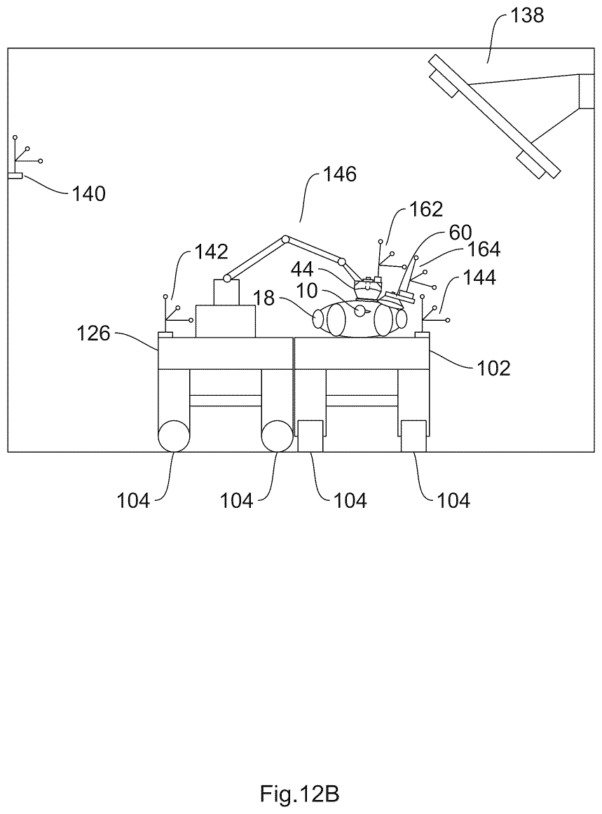

Referring to FIGS. 12A-12B, another embodiment may incorporate separate real-time or near-realtime imaging to assist in an image-guided intervention. An ultrasound imaging system (60) similar to those described in reference to FIGS. 5 and 6A may be integrated to provide additional information pertaining to the targeted tissue structure and interventional site, and may be positioned/oriented and reposition/reoriented as needed by manual operation, or via electromechanical techniques (such as via electromechanical manipulator, such as an additional robotic arm, not shown). Referring to FIG. 12B, a tracking fiducial (164) may be coupled to the ultrasound imaging head (60) to assist in registering the images produced from this subsystem with other registered imagery pertinent to the intervention.

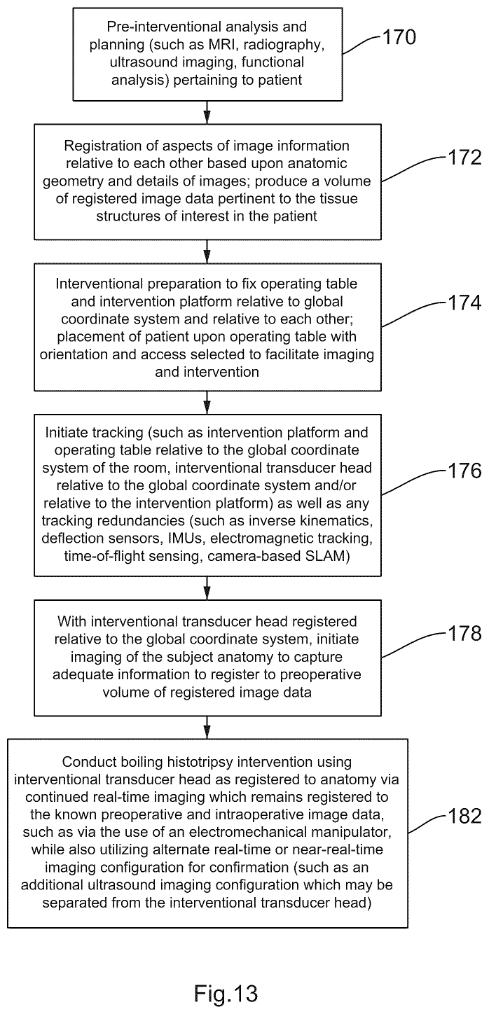

Thus referring to FIG. 13, a configuration similar to that of FIG. 11 is illustrated, with exception that after the interventional transducer head (44) and other pertinent structures and image data have been registered and made read for intervention (178), the interventional team may conduct boiling histotripsy intervention using an interventional transducer head as registered to anatomy via continued real-time imaging which remains registered to the known preoperative and intraoperative image data, such as via the use of an electromechanical manipulator, while also utilizing alternate real-time or near-real-time imaging configuration for confirmation (such as an additional ultrasound imaging configuration which may be separated from the interventional transducer head) (182).

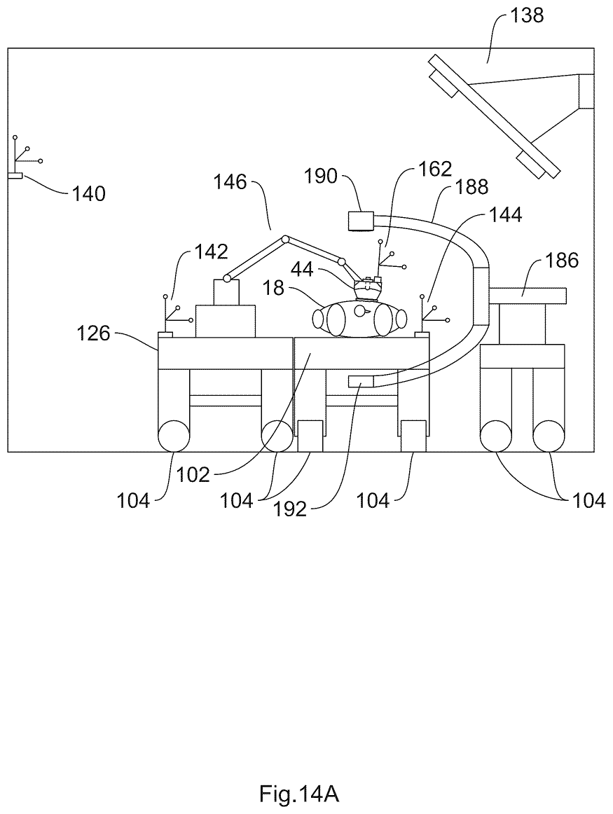

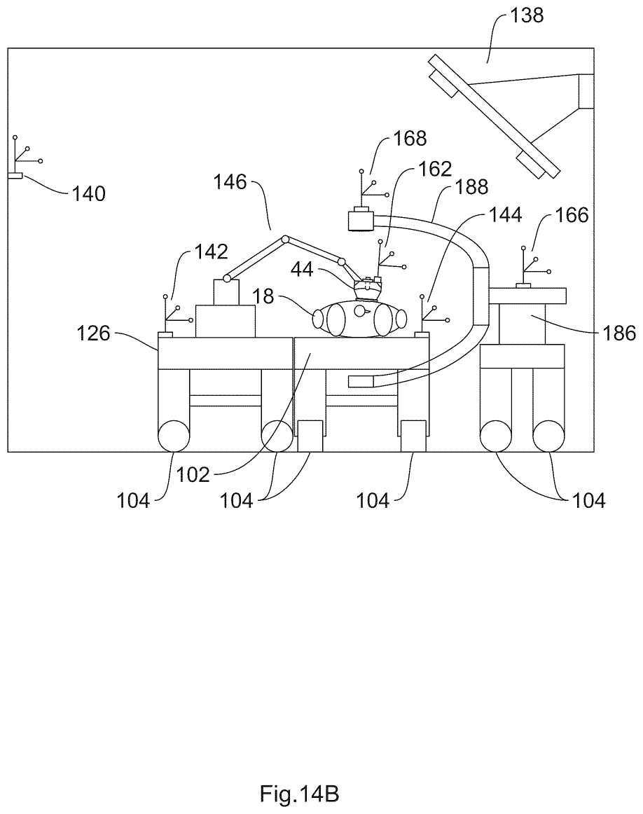

Referring to FIGS. 14A and 14B, additional real or near-real time image information may additionally or alternatively be provided using radiography and/or fluoroscopy techniques which may employ, for example, a system known as a “C-arm”, which features a radiography source (190) coupled to a sensor (192) using a “C-arm” structure (188), which may be movably and controllably coupled to a C-arm base structure (186) which may be configured to be wheeled in and locked into position (104). To assist in registering the imagery produced from the radiography of the scenario, aspects of the C-arm assembly may be tracked relative to other structures and coordinate systems, such as via one or more optical tracking fiducials (168, 166), which may be coupled to pertinent structures. Other inputs pertaining to the C-arm assembly may also be utilized in tracking pertinent structures and registering imagery, such as knowledge of the kinematics and geometry of the C-arm structures and understanding of the joint positions that pertain to position or orientation of the various components (such as the roll axis of the C-arm relative to the base 186).

Thus in a manner somewhat akin to the configuration of FIG. 13, after the interventional transducer head (44) and other pertinent structures and image data have been registered and made read for intervention (178), the interventional team may conduct boiling histotripsy intervention using an interventional transducer head as registered to anatomy via continued real-time imaging which remains registered to the known preoperative and intraoperative image data, such as via the use of an electromechanical manipulator, while also utilizing alternate real-time or near-real-time imaging configuration for confirmation (such as an additional imaging configuration which may include radiography and/or fluoroscopy, and which may be separated from the interventional transducer head) (184).

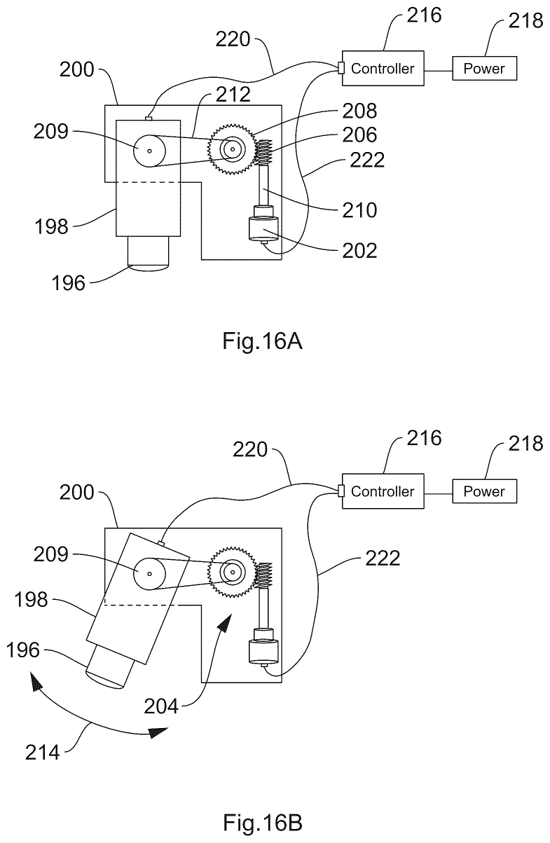

Referring to FIGS. 16A and 16B, an electromechanical sub-system may be utilized to controllably oscillate or cycle the orientation of an ultrasound imaging transducer in a controllable manner such that an associated housing may be fixedly coupled or held in place while the imaging ultrasound transducer (196) is cycled through a “volume” of tissue in the form of various discrete “slices” of ultrasound data which may be assembled, examined, and registered to other data and structures. Referring to FIG. 16A, a main housing (200) may be rotatably coupled to a housing (198) for the ultrasound imaging transducer (196). A drive motor (202) may be fixedly attached to the main housing (200) and may be configured to turnr a shaft (210) which is coupled to a lead or ball screw (206) which is configured to precisely interface with a sprocket (208), which may be operatively coupled (such as via a drive belt 212) to a pulley (209) coupled to controllably reorient the transducer housing (198) and thereby the ultrasound imaging transducer (196), such as in a cyclical manner (214) which may be prescribed by an operator and selected to provide near-real-time image data pertaining to a selected group of ultrasound image “slices”, or a “volume” image assembled from such slices (the imaging ultrasound transducer 196 and drive motor 222, for example, may be operatively coupled, such as via wire leads 220, 222, to a controller or computer (216) and intercoupled power supply (218). Such a configuration may be deemed a “scanning ultrasound imaging” or “scanning volume ultrasound imaging” configuration, for example.

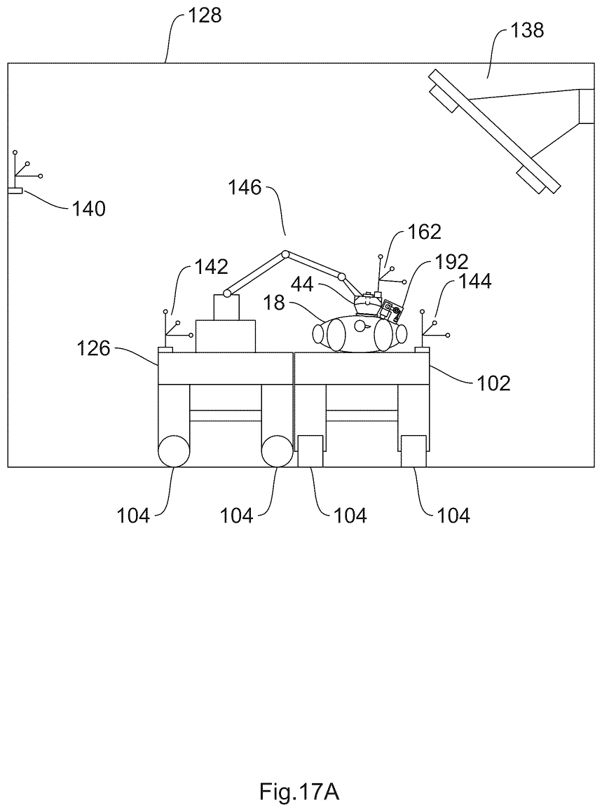

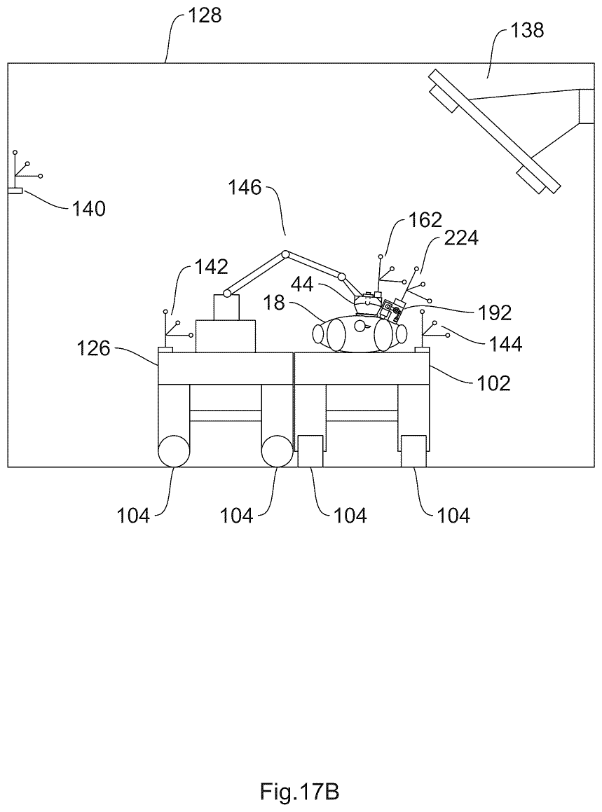

Referring to FIGS. 17A and 17B, a scanning ultrasound imaging configuration (192) is shown integrated into an operative configuration. The embodiment of FIG. 17B illustrates that the scanning ultrasound imaging configuration (192) may be coupled to a tracking fiducial (224), such as an optical tracking fiducial, so that the image data coming therefrom may be registered with other data and structures pertinent to the interventional setup.

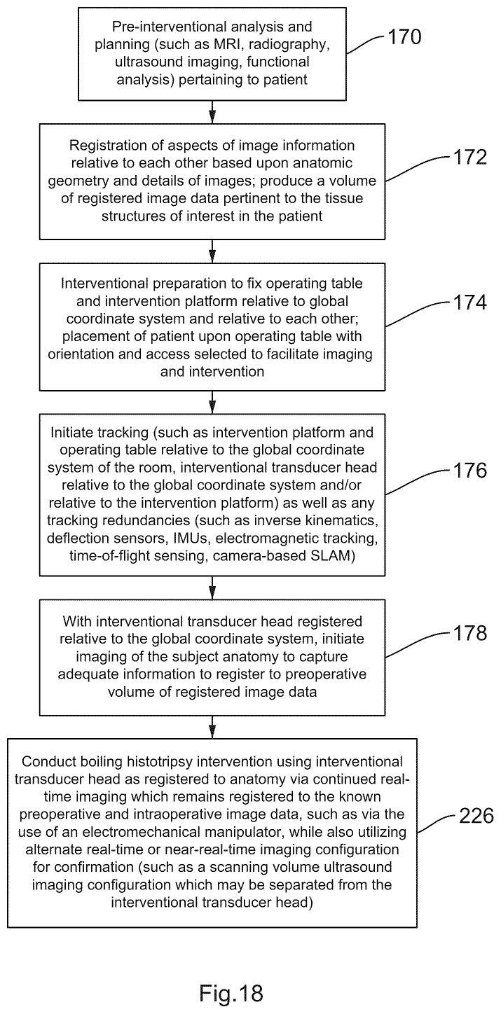

Thus referring to FIG. 18, in a manner somewhat akin to the configuration of FIG. 13, after the interventional transducer head (44) and other pertinent structures and image data have been registered and made read for intervention (178), the interventional team may conduct boiling histotripsy intervention using an interventional transducer head as registered to anatomy via continued real-time imaging which remains registered to the known preoperative and intraoperative image data, such as via the use of an electromechanical manipulator, while also utilizing alternate real-time or near-real-time imaging configuration for confirmation (such as a scanning volume ultrasound imaging configuration which may be separated from the interventional transducer head) (226).

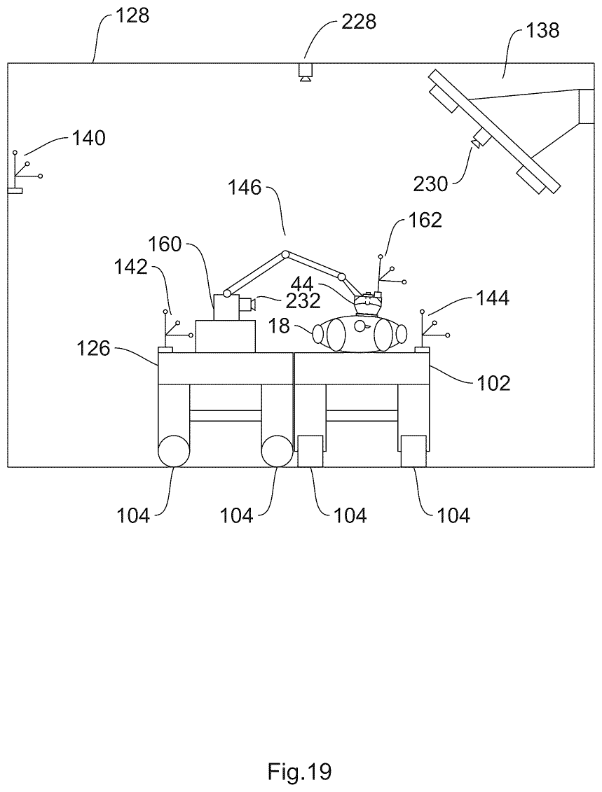

Referring to FIG. 19, additional image capture devices (228, 230, 232), such as cameras, which may be configured, for example, to operate in a visible and/or infrared spectrum for image capture, may be coupled to various aspects of the interventional configuration to provide additional information to the system and operators, such as un-obstructed views, close-in views for automated computer-vision-based analysis of repositioning and/or reorientation of particular structures relative to each other, for error analysis, optical calibration, thermal mapping and detection, and the like. For example, these image capture devices (228, 230, 232) may be utilized to assist in pose (i.e., image-based determination of position and/or orientation) determination/confirmation relative to pertinent coordinate frames and structures, and in association with SLAM-based techniques for mapping, tracking, and pose determination/confirmation.

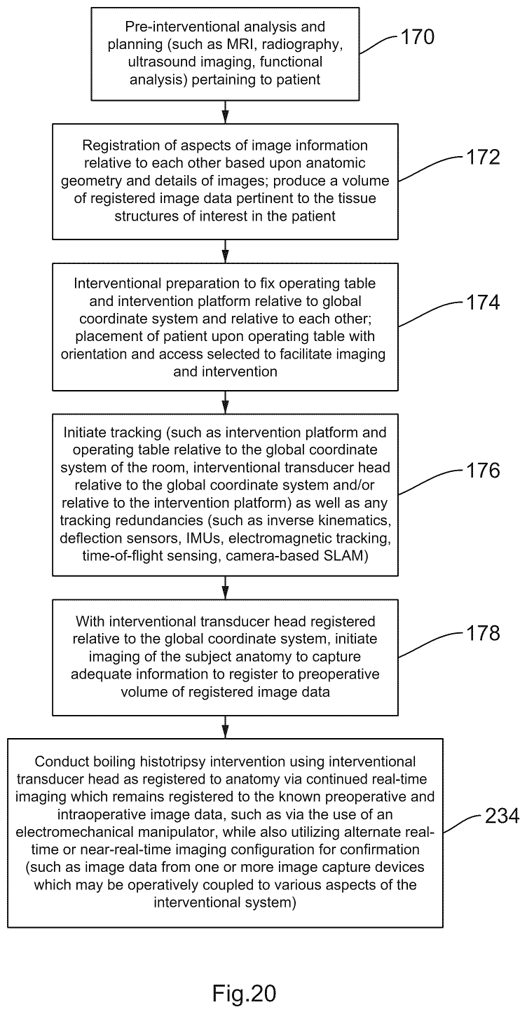

Thus referring to FIG. 20, in a manner somewhat akin to the configuration of FIG. 13, after the interventional transducer head (44) and other pertinent structures and image data have been registered and made read for intervention (178), the interventional team may conduct boiling histotripsy intervention using an interventional transducer head as registered to anatomy via continued real-time imaging which remains registered to the known preoperative and intraoperative image data, such as via the use of an electromechanical manipulator, while also utilizing alternate real-time or near-real-time imaging configuration for confirmation (such as a image data from one or more image capture devices which may be operatively coupled to various aspects of the interventional system) (234).

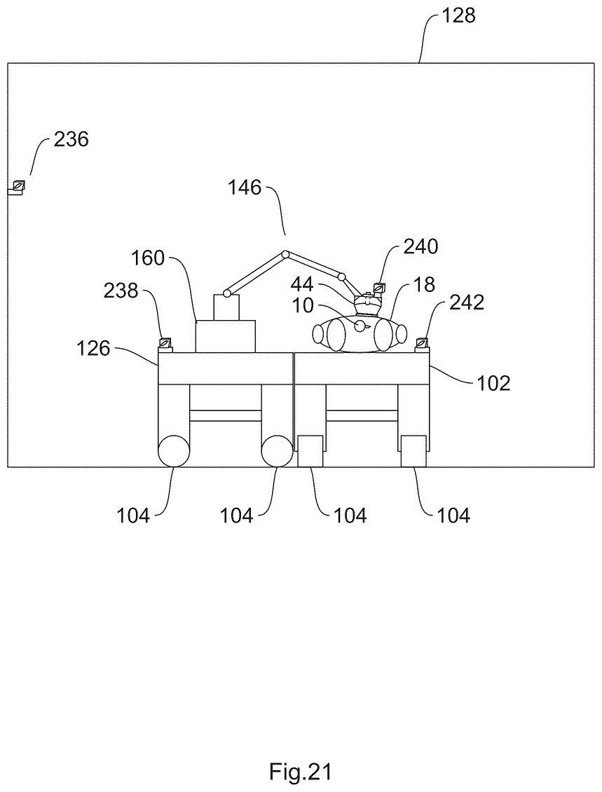

Referring to FIG. 21, electromagnetic flux based position and/or orientation sensors and associated systems (such as those available from Polhemus or Ascension, as noted above) may be utilized to track positions of various structures and elements relative to each other (i.e., without the use of optical tracking techniques and optical tracking based fiducials), such as the relative positions and/or orientations of the interventional transducer head (44), surgical bed (102), interventional cart (238), and operating room (236) relative to each other.

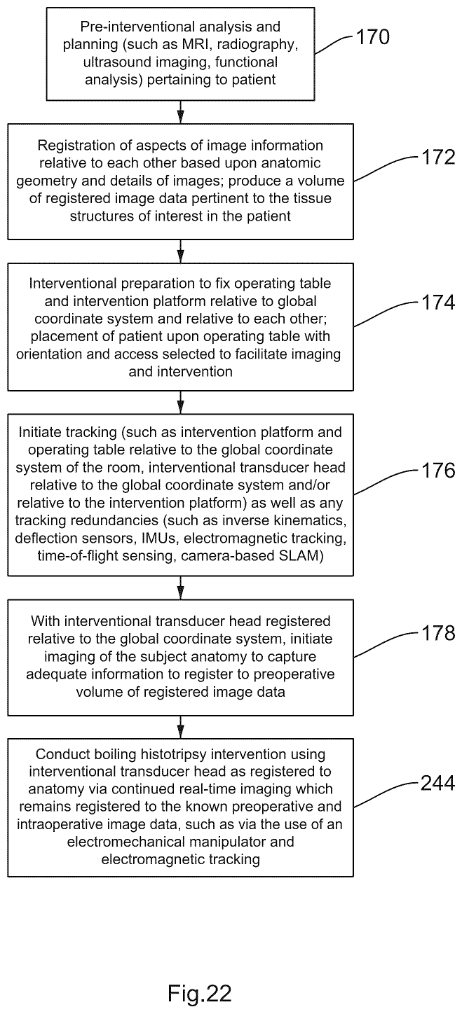

Thus referring to FIG. 22, in a manner somewhat akin to the configuration of FIG. 13, after the interventional transducer head (44) and other pertinent structures and image data have been registered and made read for intervention (178), the interventional team may conduct boiling histotripsy intervention using an interventional transducer head as registered to anatomy via continued real-time imaging which remains registered to the known preoperative and intraoperative image data, such as via the use of an electromechanical manipulator, while also utilizing alternate real-time or near-real-time imaging configuration for confirmation (such as image data from one or more image capture devices which may be operatively coupled to various aspects of the interventional system) (244).

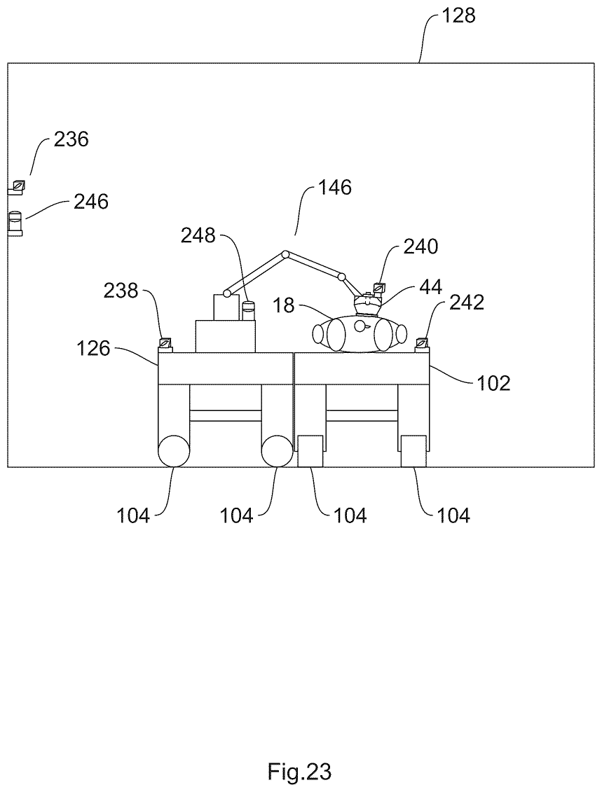

Referring to FIG. 23, time-of-flight or point-cloud sensors, such as LIDAR sensors, may be integrated into the subject systems as well, as illustrated in the embodiment of FIG. 23 wherein electromagnetic flux based tracking may be accompanied by data from one or more LIDAR sensors (246, 248) which may be positioned, oriented, and configured to assist in tracking, control, and confirmation of various structures relative to each other. For example, a LIDAR sensor (246) may be fixedly coupled to the operating room (128) to provide updated point cloud data pertaining to general movement of structures and anatomy pertaining to the intervention, which may be registered and fed into control systems; another LIDAR sensor (248) may be configured to provide registered and updated point cloud data pertaining to closer-in movement of the interventional transducer head (44), patient (18), and electromechanical manipulator components (146) relative to each other.

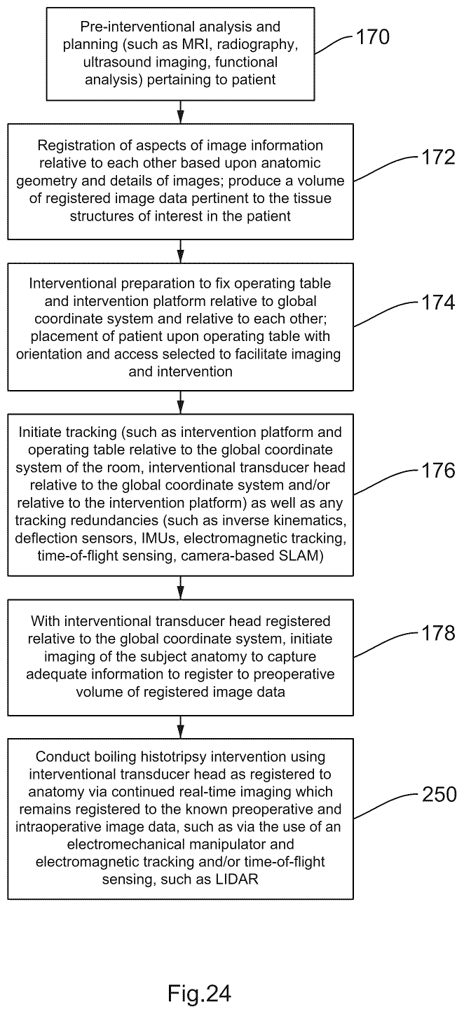

Thus referring to FIG. 24, in a manner somewhat akin to the configuration of FIG. 13, after the interventional transducer head (44) and other pertinent structures and image data have been registered and made read for intervention (178), the interventional team may conduct boiling histotripsy intervention using interventional transducer head as registered to anatomy via continued real-time imaging which remains registered to the known preoperative and intraoperative image data, such as via the use of an electromechanical manipulator and electromagnetic tracking and/or time-of-flight sensing, such as LIDAR (250).

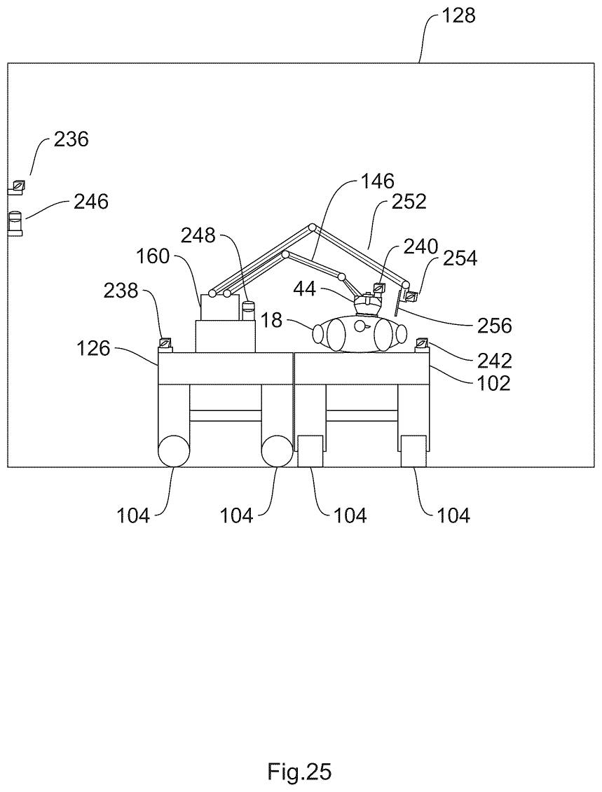

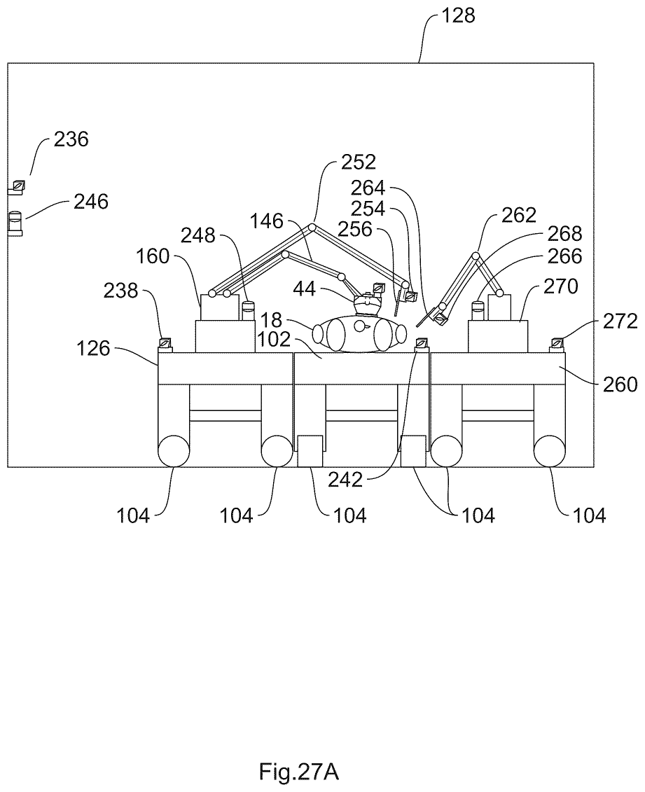

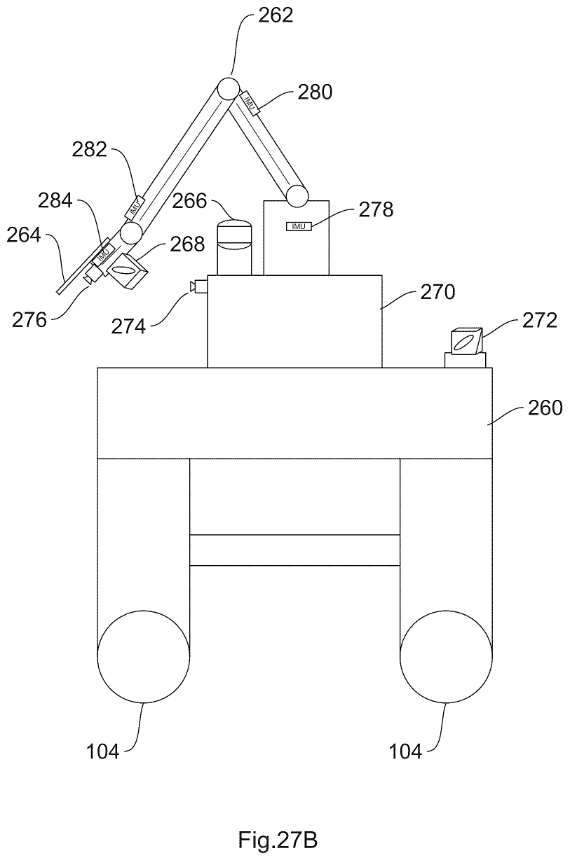

Referring to FIGS. 25 and FIGS. 27A-27B, in certain embodiments it may be useful to have precision electromechanical manipulation of more than one interventional assembly or instrument. FIG. 25 illustrates an embodiment with a separate electromechanical manipulator assembly (252; as with the first manipulator assembly 146, the second 252, or third 262 as in FIGS. 27A-27B, may be an articulated robotic arm, such as those available from Barrett or Kuka, as noted above) coupled to an interventional instrument (256) such as an aspiration needle, cannula, or catheter. As noted above, in certain embodiments it may be desirable to controllably remove material after boiling histotripsy, such as via aspiration, and preferably under image guidance. Using data from known geometric and kinematic relationship, joint positions pertaining to the manipulator assembly (252, and/or 262 in the embodiment of FIGS. 27A-27B), and tracking sensors which may be intercoupled (the embodiments of FIGS. 25 and 27A illustrate electromagnetic tracking devices 254 coupled to the distal end of the second manipulator 252, as well as an electromagnetic tracking device 268 coupled to the distal end of a third manipulator 262 of FIGS. 27A and 27B). The close-up view of FIG. 27B also shows IMU devices (278, 280, 282, 284) which may be coupled to various structures to assist in position and/or orientation change determination, in control system input and confirmation, in sensing collisions, and the like.

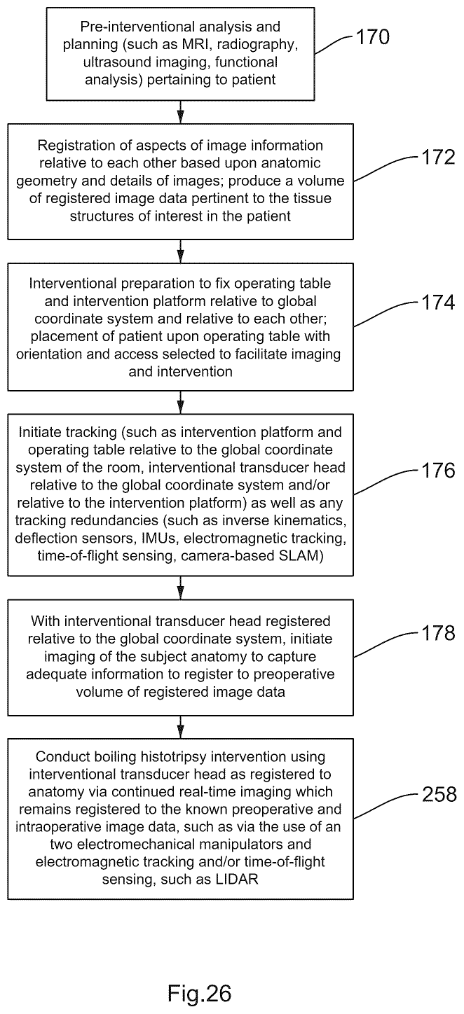

Thus referring to FIG. 26, in a manner somewhat akin to the configuration of FIG. 13, after the interventional transducer head (44) and other pertinent structures and image data have been registered and made read for intervention (178), the interventional team may conduct boiling histotripsy intervention using interventional transducer head as registered to anatomy via continued real-time imaging which remains registered to the known preoperative and intraoperative image data, such as via the use of a two electromechanical manipulators and electromagnetic tracking and/or time-of-flight sensing, such as LIDAR (258). The third manipulator assembly (262) of FIG. 27B may be utilized to carry and effect yet another interventional instrument, such as a cannula, aspiration needle, injection needle (for example, to assist in effecting a high precision injection of a medicine, such as for pain or infection management, under image guidance using coordinate system/image data registration), or imaging probe. FIG. 27B also shows additional image capture devices (274, 276) and a LIDAR sensor (266) positioned to capture point clouds, images, and generally information pertinent to the intervention. The additional interventional cart (260) also may comprise specialized braking/stability wheel assemblies (104) and may be tracked, for example by use of an electromagnetic flux based position and/or orientation sensor (272) which may be coupled thereto.

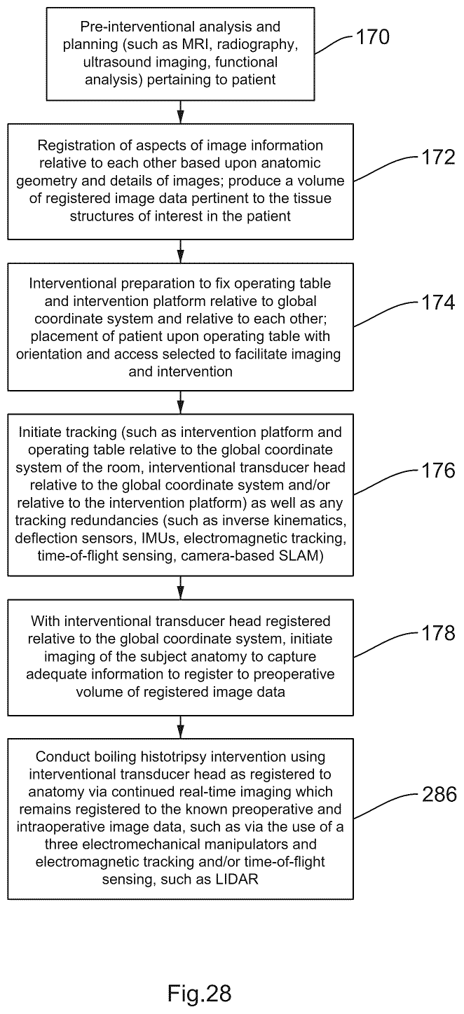

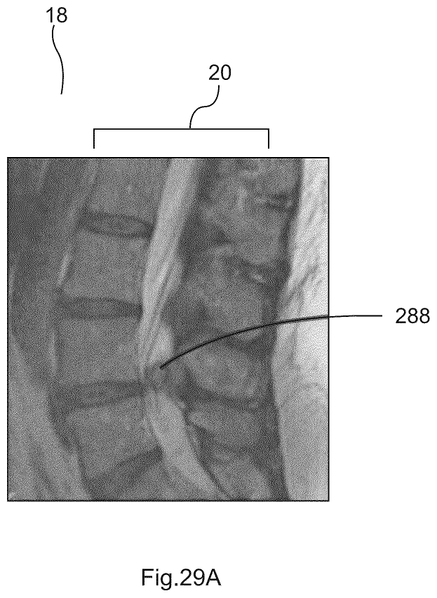

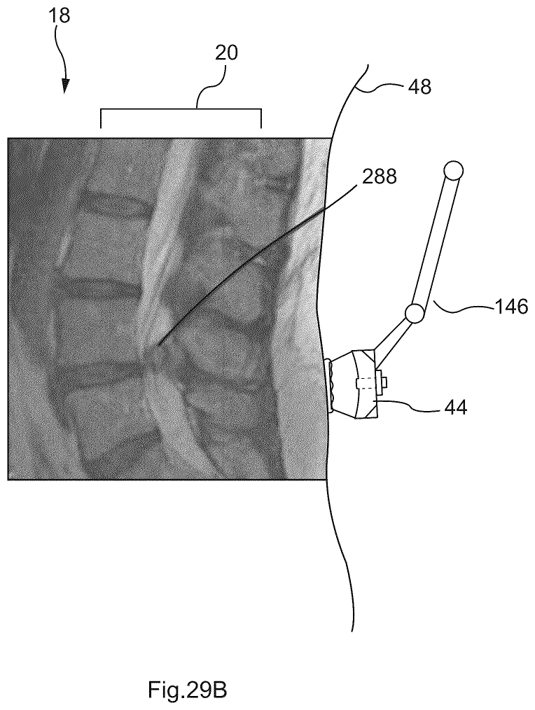

Referring to FIG. 28, in a manner somewhat akin to the configuration of FIG. 13, after the interventional transducer head (44) and other pertinent structures and image data have been registered and made read for intervention (178), the interventional team may conduct boiling histotripsy intervention using interventional transducer head as registered to anatomy via continued real-time imaging which remains registered to the known preoperative and intraoperative image data, such as via the use of a three electromechanical manipulators and electromagnetic tracking and/or time-of-flight sensing, such as LIDAR (286).

The aforementioned embodiments generally have been discussed for illustration purposes in the context of a spinal intervention for intervertebral disc herniation, but the systems described above are broadly applicable. FIGS. 29A to FIG. 57 illustrate further treatment paradigms and embodiments which may employ such systems, methods, and configurations.

Referring to FIG. 29A, removal of a spinal cyst (288) in the spine (20) of a patient (18) utilizing conventional surgical approaches can be very invasive. As shown in FIG. 29B, an interventional BH HIFU transducer head (44) may be positioned and/or oriented with image-guided precision, such as via an image-guided electromechanical manipulator (146), to emulsify and/or atomize such cyst, followed by potential image-guided aspiration and/or injection.

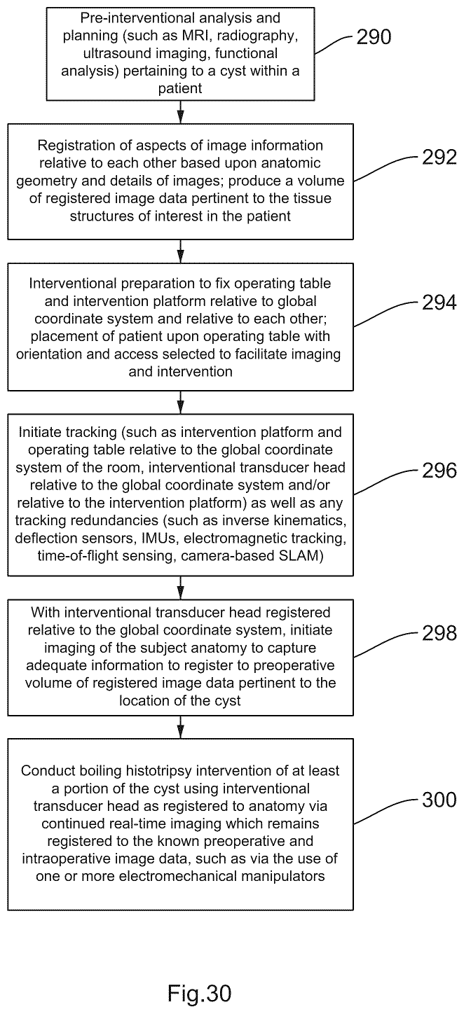

Referring to FIG. 30, pre-interventional analysis and planning (such as MRI, radiography, ultrasound imaging, functional analysis) pertaining to a cyst within a patient may be conducted (290), followed by registration of aspects of image information relative to each other based upon anatomic geometry and details of images; registered image data pertinent to the tissue structures of interest in the patient may be produced (292). Interventional preparation may be conducted to fix an operating table and an intervention platform relative to global coordinate system and relative to each other; placement of patient upon operating table with orientation and access selected to facilitate imaging and intervention may be conducted (294). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (296). With interventional transducer head registered relative to the global coordinate system, imaging of the subject anatomy may be initiated to capture adequate information to register to preoperative volume of registered image data pertinent to the location of the cyst (298). The interventional team may conduct boiling histotripsy intervention of at least a portion of the cyst using interventional transducer head as registered to anatomy via continued real-time imaging which remains registered to the known preoperative and intraoperative image data, such as via the use of one or more electromechanical manipulators (300).

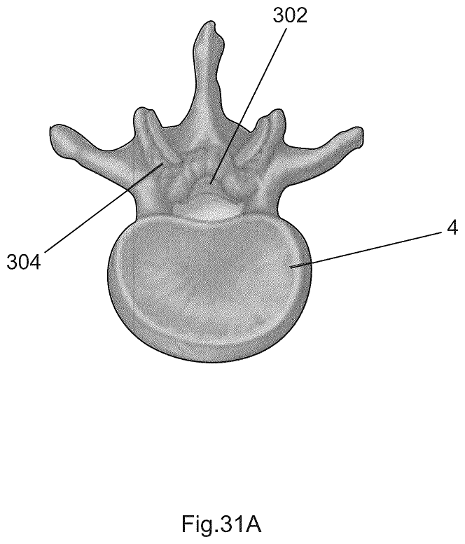

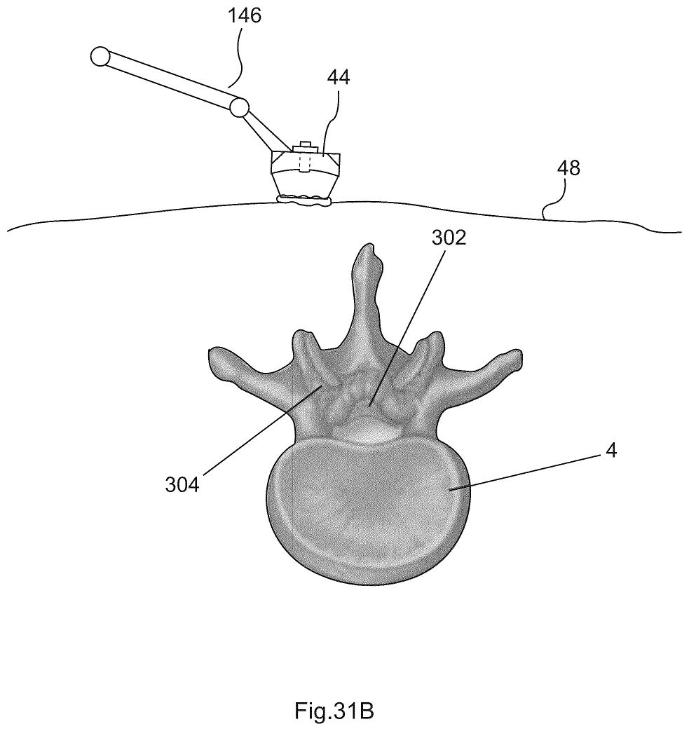

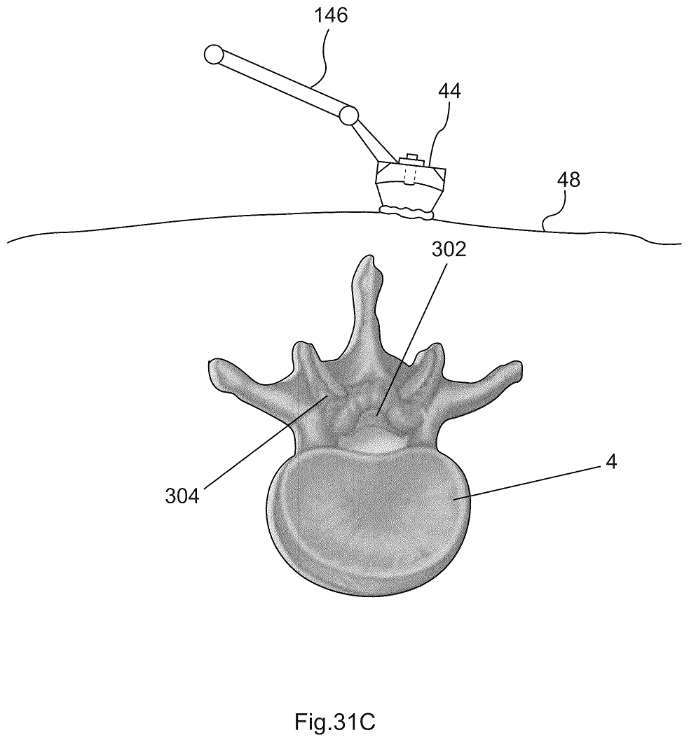

Referring to FIG. 31A, a hypertrophy or other aberration of the geometry of a ligamentum flavum (302) or facet joint (304) in the spine (20) of a patient (18) may cause significant problems, and utilizing conventional surgical approaches can be very invasive. As shown in FIG. 31B, an interventional BH HIFU transducer head (44) may be positioned and/or oriented with image-guided precision, such as via an image-guided electromechanical manipulator (146), to emulsify and/or atomize such aspects of a targeted facet joint (304) articular and/or connective tissue structure, followed by potential image-guided aspiration and/or injection. As shown in FIG. 31C, an interventional BH HIFU transducer head (44) may be positioned and/or oriented with image-guided precision, such as via an image-guided electromechanical manipulator (146), to emulsify and/or atomize such aspects of a targeted portion of a ligamentum flavum (302) connective tissue structure, followed by potential image-guided aspiration and/or injection.

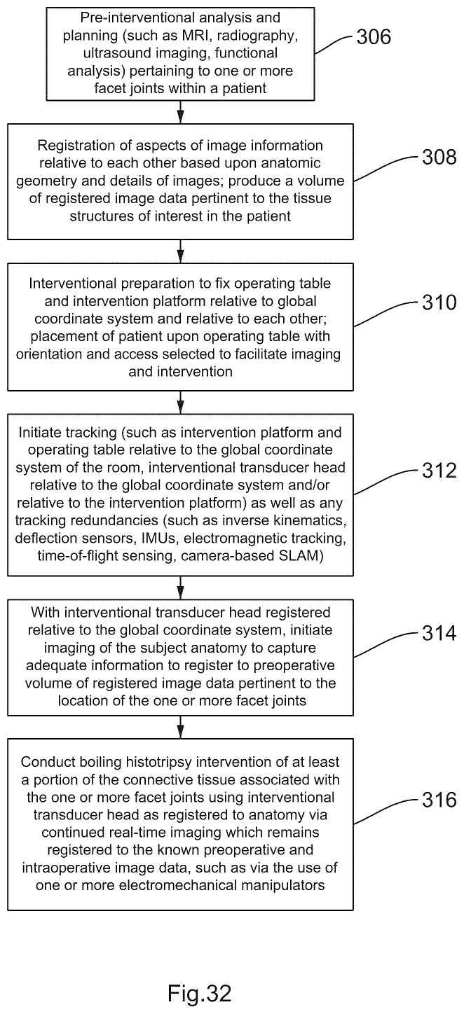

Referring to FIG. 32, pre-interventional analysis and planning (such as MRI, radiography, ultrasound imaging, functional analysis) pertaining to one or more facet joints within a patient may be conducted (306), followed by registration of aspects of image information relative to each other based upon anatomic geometry and details of images; registered image data pertinent to the tissue structures of interest in the patient may be produced (308). Interventional preparation may be conducted to fix an operating table and an intervention platform relative to global coordinate system and relative to each other; placement of patient upon operating table with orientation and access selected to facilitate imaging and intervention may be conducted (310). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (312). With interventional transducer head registered relative to the global coordinate system, imaging of the subject anatomy may be initiated to capture adequate information to register to preoperative volume of registered image data pertinent to the location of the one or more facet joints (314). The interventional team may conduct boiling histotripsy intervention of at least a portion of the one or more facet joints using interventional transducer head as registered to anatomy via continued real-time imaging which remains registered to the known preoperative and intraoperative image data, such as via the use of one or more electromechanical manipulators (316).

Image-guided ultrasonic radiation from subject configurations may also be utilized to conduct one or more nerve blocks or denervation procedures, such as a medial branch block in the vicinity of a facet joint of the spine.

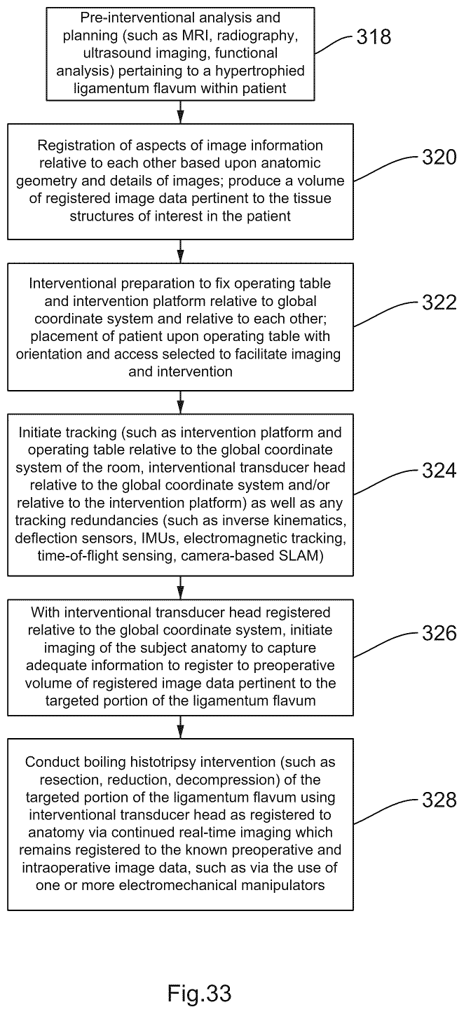

Referring to FIG. 33, pre-interventional analysis and planning (such as MRI, radiography, ultrasound imaging, functional analysis) pertaining to an aspect of the ligamentum flavum within a patient may be conducted (318), followed by registration of aspects of image information relative to each other based upon anatomic geometry and details of images; registered image data pertinent to the tissue structures of interest in the patient may be produced (320). Interventional preparation may be conducted to fix an operating table and an intervention platform relative to global coordinate system and relative to each other; placement of patient upon operating table with orientation and access selected to facilitate imaging and intervention may be conducted (322). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (324). With interventional transducer head registered relative to the global coordinate system, imaging of the subject anatomy may be initiated to capture adequate information to register to preoperative volume of registered image data pertinent to the location of the ligamentum flavum (326). The interventional team may conduct boiling histotripsy intervention of at least a portion of the ligamentum flavum using interventional transducer head as registered to anatomy via continued real-time imaging which remains registered to the known preoperative and intraoperative image data, such as via the use of one or more electromechanical manipulators (328).

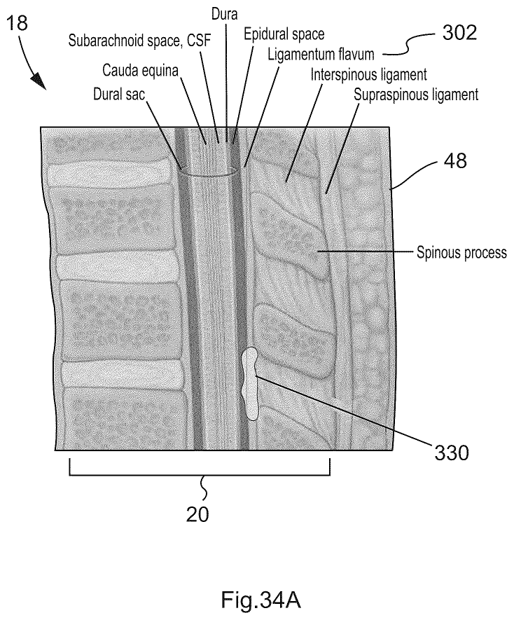

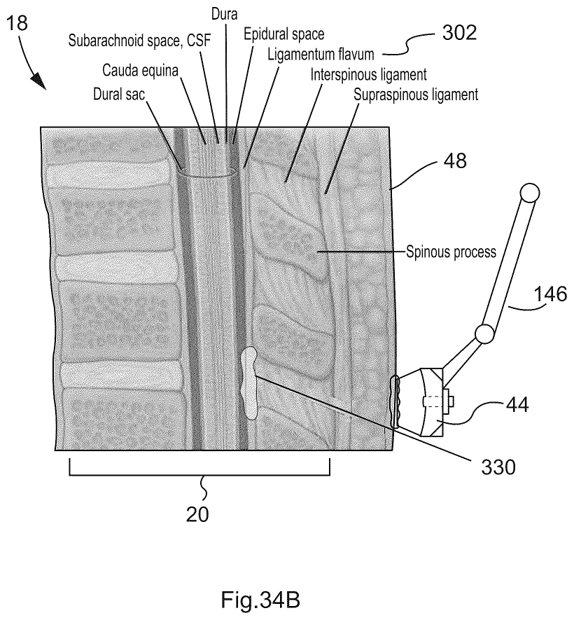

Referring to FIG. 34A, an epidural tumor (330) in the spine (20) of a patient (18) may cause significant problems, and utilizing conventional surgical approaches can be very invasive. As shown in FIG. 34B, an interventional BH HIFU transducer head (44) may be positioned and/or oriented with image-guided precision, such as via an image-guided electromechanical manipulator (146), to emulsify and/or atomize such aspects of an epidural tumor (330), followed by potential image-guided aspiration and/or injection.

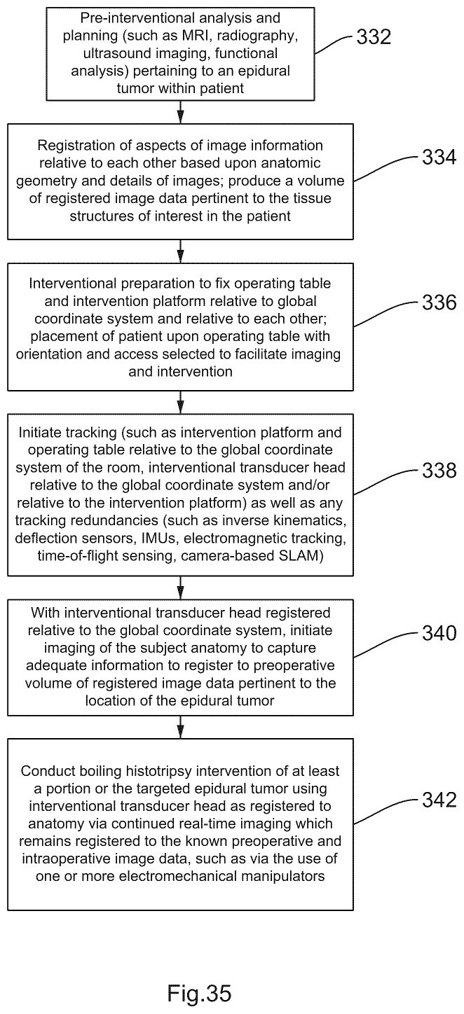

Referring to FIG. 35, pre-interventional analysis and planning (such as MRI, radiography, ultrasound imaging, functional analysis) pertaining to an aspect of an epidural tumor within a patient may be conducted (332), followed by registration of aspects of image information relative to each other based upon anatomic geometry and details of images; registered image data pertinent to the tissue structures of interest in the patient may be produced (334). Interventional preparation may be conducted to fix an operating table and an intervention platform relative to global coordinate system and relative to each other; placement of patient upon operating table with orientation and access selected to facilitate imaging and intervention may be conducted (336). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (338). With interventional transducer head registered relative to the global coordinate system, imaging of the subject anatomy may be initiated to capture adequate information to register to preoperative volume of registered image data pertinent to the location of the epidural tumor (340). The interventional team may conduct boiling histotripsy intervention of at least a portion of the epidural tumor using interventional transducer head as registered to anatomy via continued real-time imaging which remains registered to the known preoperative and intraoperative image data, such as via the use of one or more electromechanical manipulators (342).

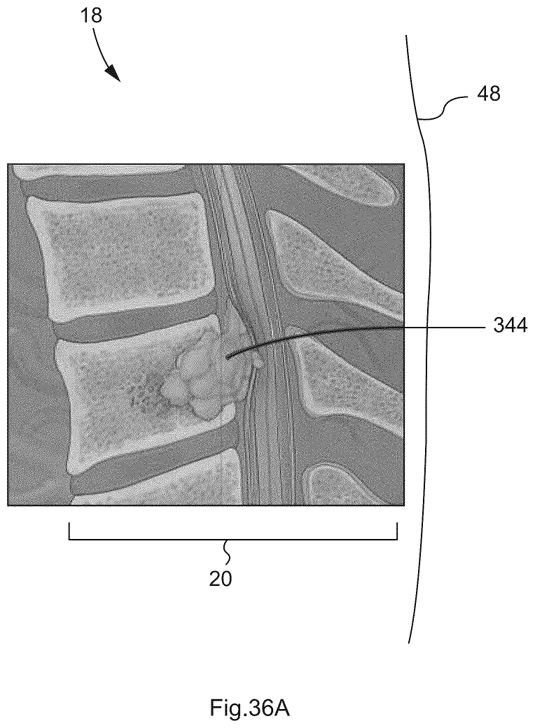

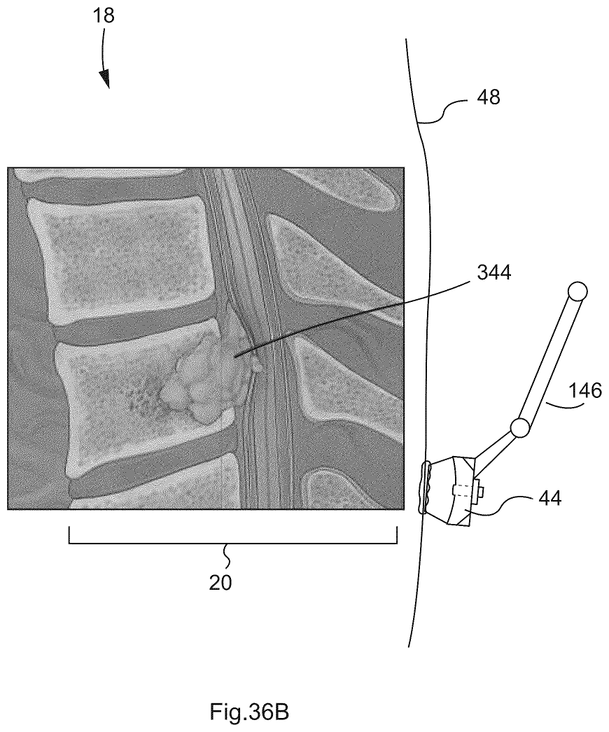

Referring to FIG. 36A, a metastatic spine tumor (344) in the spine (20) of a patient (18) may cause significant problems, and utilizing conventional surgical approaches can be very invasive. As shown in FIG. 36B, an interventional BH HIFU transducer head (44) may be positioned and/or oriented with image-guided precision, such as via an image-guided electromechanical manipulator (146), to emulsify and/or atomize such aspects of a metastatic spine tumor (344), followed by potential image-guided aspiration and/or injection.

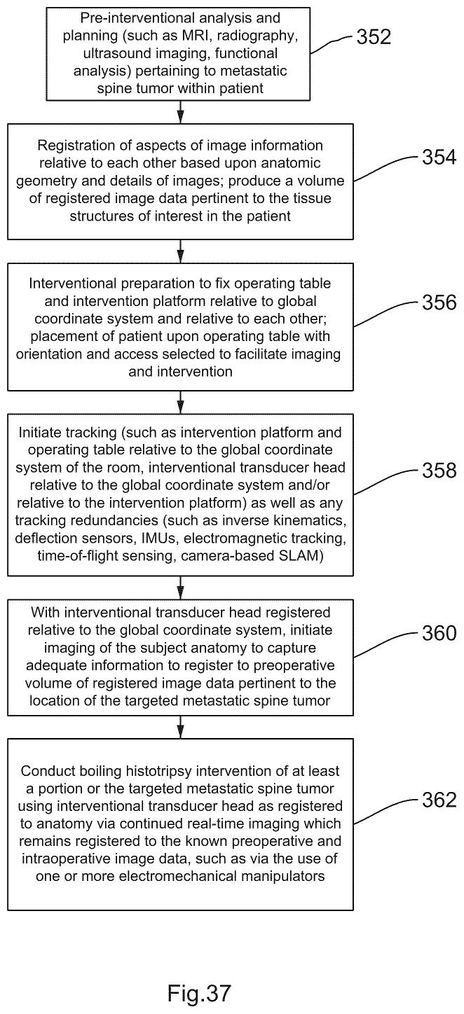

Referring to FIG. 37, pre-interventional analysis and planning (such as MRI, radiography, ultrasound imaging, functional analysis) pertaining to an aspect of a metastatic spine tumor within a patient may be conducted (352), followed by registration of aspects of image information relative to each other based upon anatomic geometry and details of images; registered image data pertinent to the tissue structures of interest in the patient may be produced (354). Interventional preparation may be conducted to fix an operating table and an intervention platform relative to global coordinate system and relative to each other; placement of patient upon operating table with orientation and access selected to facilitate imaging and intervention may be conducted (356). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (358). With interventional transducer head registered relative to the global coordinate system, imaging of the subject anatomy may be initiated to capture adequate information to register to preoperative volume of registered image data pertinent to the location of the metastatic spine tumor (360). The interventional team may conduct boiling histotripsy intervention of at least a portion of the metastatic spine tumor using interventional transducer head as registered to anatomy via continued real-time imaging which remains registered to the known preoperative and intraoperative image data, such as via the use of one or more electromechanical manipulators (362).

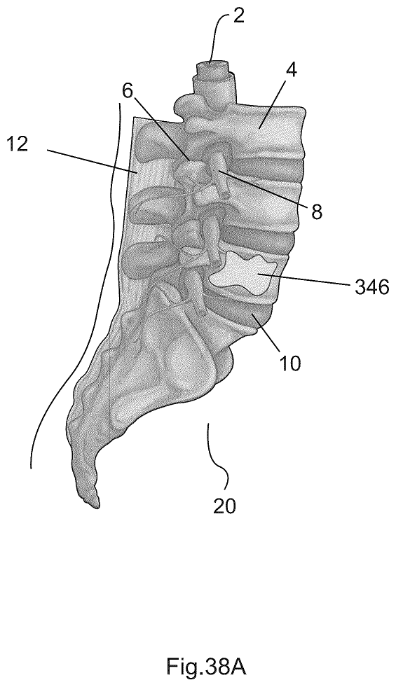

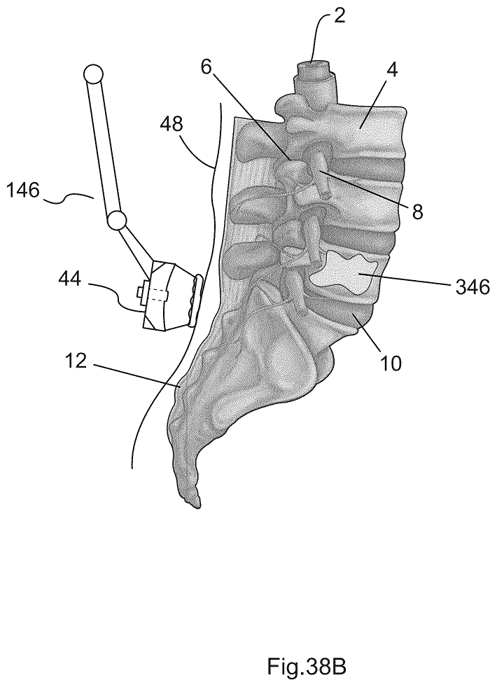

Referring to FIG. 38A, a spine-associated sarcoma (346) in the spine (20) of a patient (18) may cause significant problems, and utilizing conventional surgical approaches can be very invasive. As shown in FIG. 38B, an interventional BH HIFU transducer head (44) may be positioned and/or oriented with image-guided precision, such as via an image-guided electromechanical manipulator (146), to emulsify and/or atomize such aspects of a spine-associated sarcoma (346), followed by potential image-guided aspiration and/or injection.

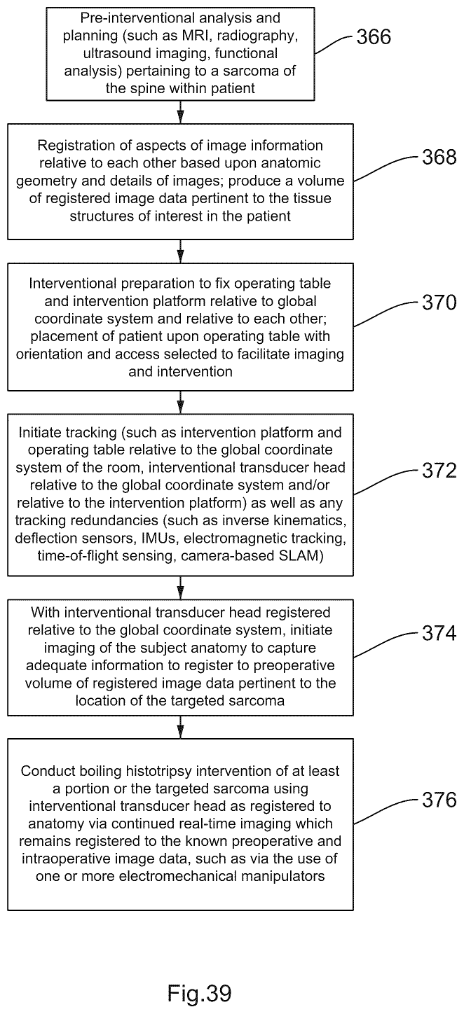

Referring to FIG. 39, pre-interventional analysis and planning (such as MRI, radiography, ultrasound imaging, functional analysis) pertaining to an aspect of a sarcoma within a patient may be conducted (366), followed by registration of aspects of image information relative to each other based upon anatomic geometry and details of images; registered image data pertinent to the tissue structures of interest in the patient may be produced (368). Interventional preparation may be conducted to fix an operating table and an intervention platform relative to global coordinate system and relative to each other; placement of patient upon operating table with orientation and access selected to facilitate imaging and intervention may be conducted (370). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (372). With interventional transducer head registered relative to the global coordinate system, imaging of the subject anatomy may be initiated to capture adequate information to register to preoperative volume of registered image data pertinent to the location of the targeted sarcoma (374). The interventional team may conduct boiling histotripsy intervention of at least a portion of the targeted sarcoma using interventional transducer head as registered to anatomy via continued real-time imaging which remains registered to the known preoperative and intraoperative image data, such as via the use of one or more electromechanical manipulators (376).

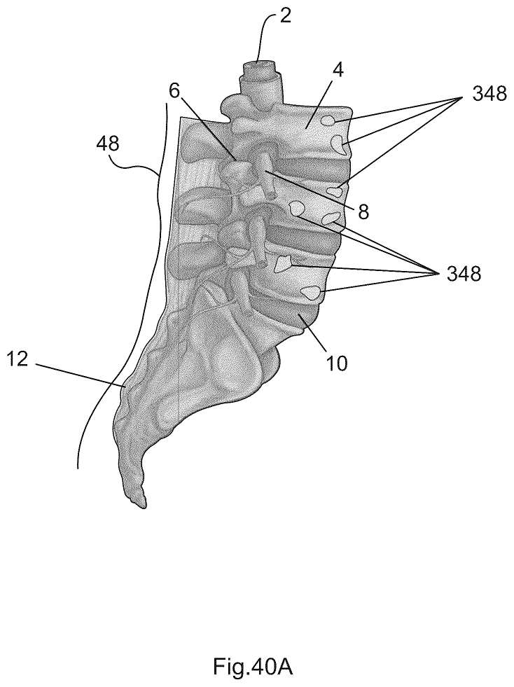

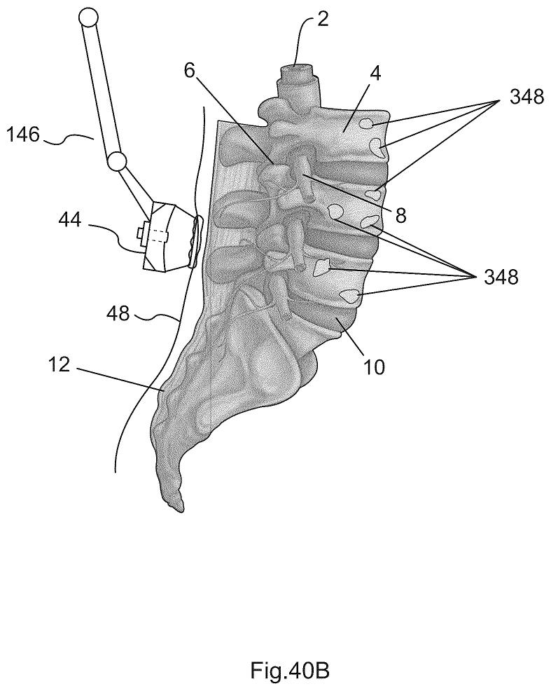

Referring to FIG. 40A, one or more spine-associated myelomas (348) in the spine (20) of a patient (18) may cause significant problems, and utilizing conventional surgical approaches can be very invasive. As shown in FIG. 40B, an interventional BH HIFU transducer head (44) may be positioned and/or oriented with image-guided precision, such as via an image-guided electromechanical manipulator (146), to emulsify and/or atomize such aspects of the one or more spine-associated myelomas (348), followed by potential image-guided aspiration and/or injection.

Referring to FIG. 39, pre-interventional analysis and planning (such as MRI, radiography, ultrasound imaging, functional analysis) pertaining to an aspect of at least one myeloma within a patient may be conducted (380), followed by registration of aspects of image information relative to each other based upon anatomic geometry and details of images; registered image data pertinent to the tissue structures of interest in the patient may be produced (382). Interventional preparation may be conducted to fix an operating table and an intervention platform relative to global coordinate system and relative to each other; placement of patient upon operating table with orientation and access selected to facilitate imaging and intervention may be conducted (384). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (386). With interventional transducer head registered relative to the global coordinate system, imaging of the subject anatomy may be initiated to capture adequate information to register to preoperative volume of registered image data pertinent to the location of the targeted myeloma (388). The interventional team may conduct boiling histotripsy intervention of at least a portion of the targeted myeloma using interventional transducer head as registered to anatomy via continued real-time imaging which remains registered to the known preoperative and intraoperative image data, such as via the use of one or more electromechanical manipulators (390).

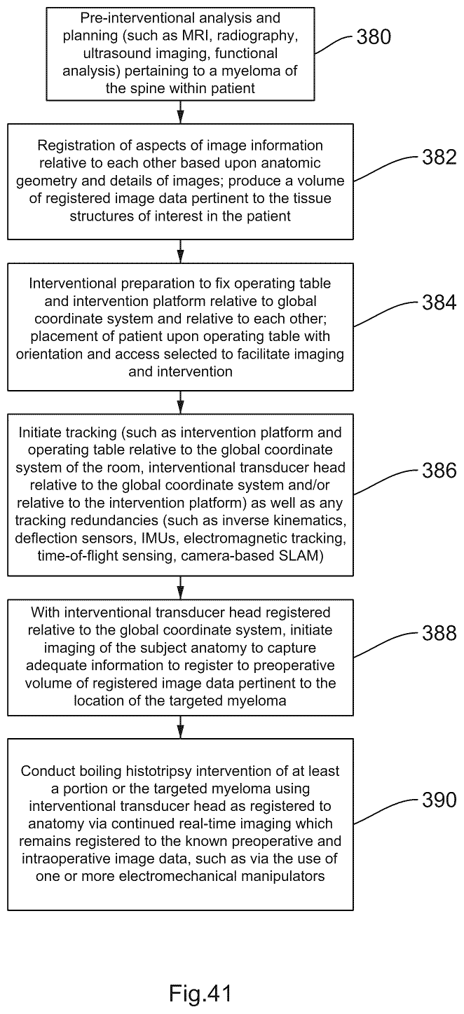

Referring to FIG. 41, a registered intervention configuration is illustrated wherein pre-interventional analysis and planning (such as MRI, radiography, ultrasound imaging, functional analysis) may be conducted pertaining to a myeloma of the spine within patient (380). Registration of aspects of image information relative to each other may be conducted based upon anatomic geometry and details of images, and a volume of registered image data pertinent to the tissue structures of interest in the patient may be produced (382). Interventional preparation may be conducted to fix operating table and intervention platform relative to global coordinate system and relative to each other; placement of the patient upon operating table may be conducted with orientation and access selected to facilitate imaging and intervention (384). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (386). With the interventional transducer head registered relative to the global coordinate system, imaging of the subject anatomy may be initiated to capture adequate information to register to preoperative volume of registered image data pertinent to the location of the targeted myeloma (388). Boiling histotripsy intervention may be conducted of at least a portion of the targeted myeloma using interventional transducer head as registered to anatomy via continued real-time imaging which remains registered to the known preoperative and intraoperative image data, such as via the use of one or more electromechanical manipulators (390).

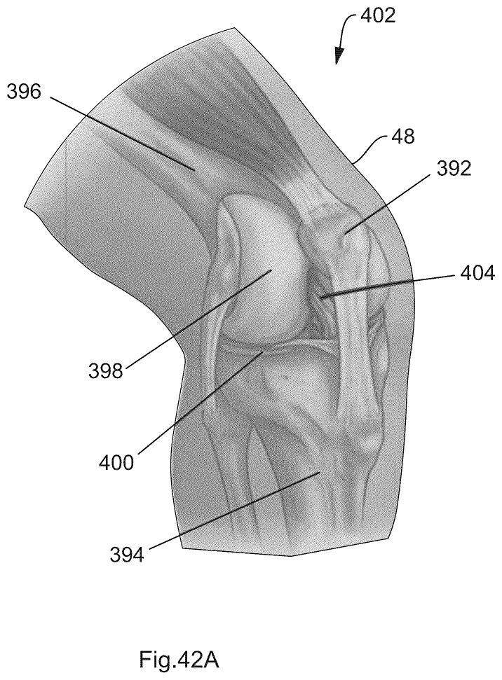

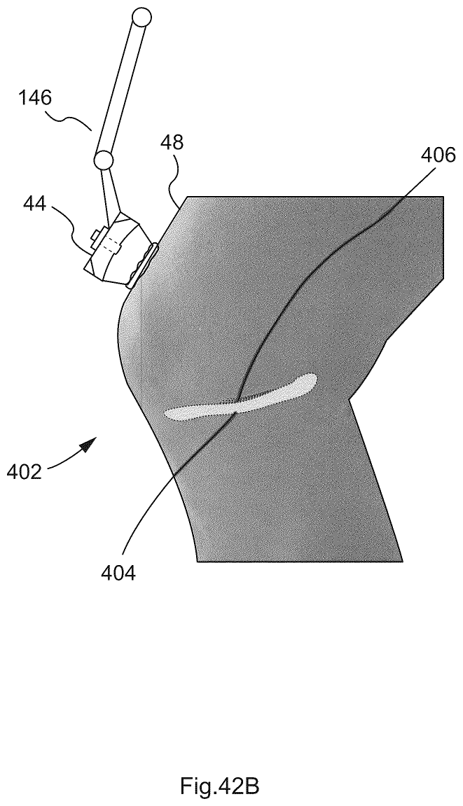

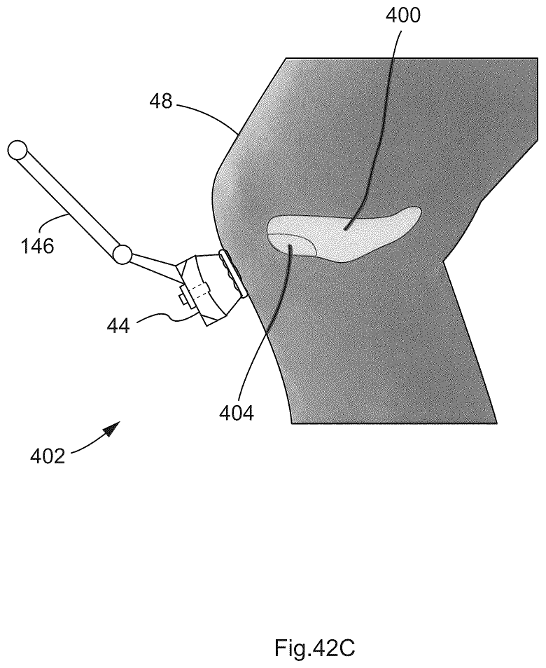

Referring to FIG. 42A, various structures of the knee (402) of a patient (18) are shown, including the femur (396), patella (392), anterior cruciate ligament (404), femoral articular cartilage (398), meniscus (400), and tibia (394). Injuries to various structures of the knee, such as to the anterior cruciate ligament (“ACL”) or meniscus, or other ligaments, tendons, or structures, may cause significant problems, and utilizing conventional surgical approaches can be very invasive. As shown in FIGS. 42B and 42C, an interventional BH HIFU transducer head (44) may be positioned and/or oriented with image-guided precision, such as via an image-guided electromechanical manipulator (146), to emulsify and/or atomize such aspects of structures such as frayed or damaged portions (406, 408, respectively) of an ACL (404) or meniscus (400), followed by potential image-guided aspiration and/or injection.

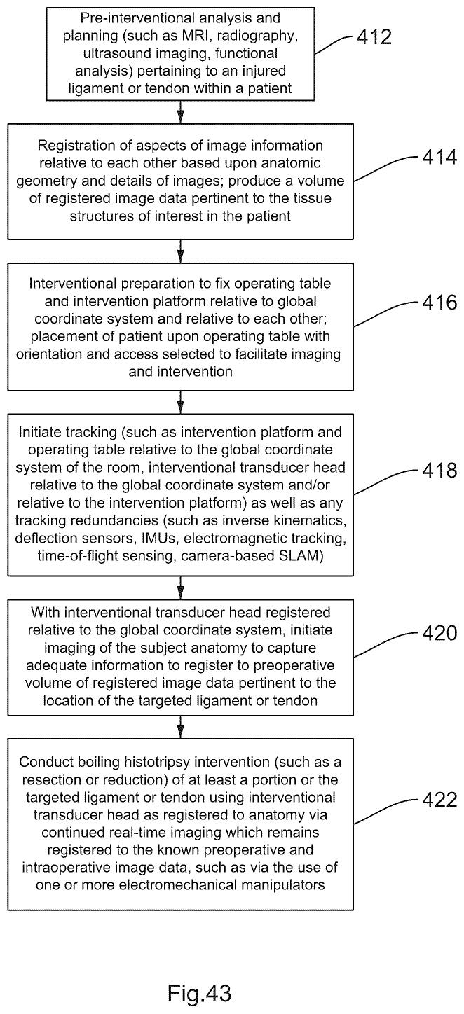

Referring to FIG. 43, pre-interventional analysis and planning (such as MRI, radiography, ultrasound imaging, functional analysis) pertaining to an aspect of at least one injured ligament or tendon within a patient may be conducted (412), followed by registration of aspects of image information relative to each other based upon anatomic geometry and details of images; registered image data pertinent to the tissue structures of interest in the patient may be produced (414). Interventional preparation may be conducted to fix an operating table and an intervention platform relative to global coordinate system and relative to each other; placement of patient upon operating table with orientation and access selected to facilitate imaging and intervention may be conducted (416). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (418). With interventional transducer head registered relative to the global coordinate system, imaging of the subject anatomy may be initiated to capture adequate information to register to preoperative volume of registered image data pertinent to the location of the targeted ligament or tendon (420). The interventional team may conduct boiling histotripsy intervention of at least a portion of the targeted ligament or tendon using interventional transducer head as registered to anatomy via continued real-time imaging which remains registered to the known preoperative and intraoperative image data, such as via the use of one or more electromechanical manipulators (422).

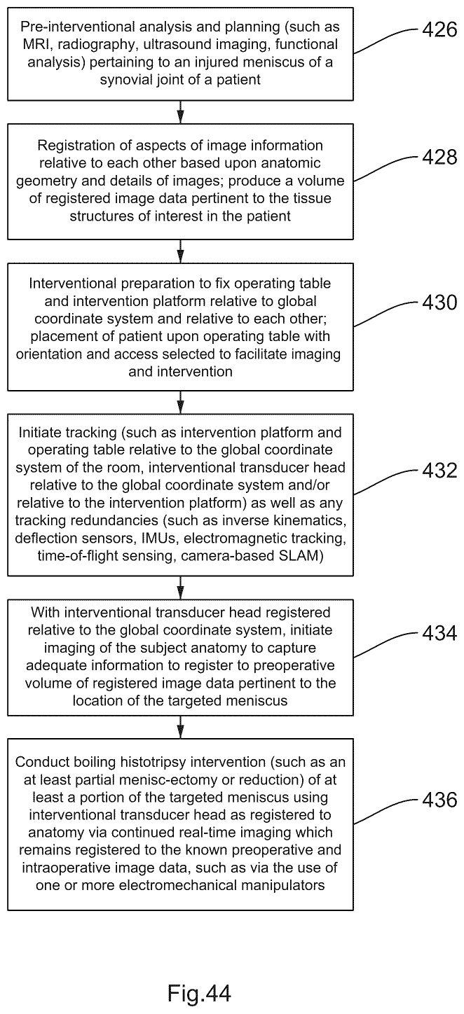

Referring to FIG. 44, pre-interventional analysis and planning (such as MRI, radiography, ultrasound imaging, functional analysis) pertaining to an aspect of a meniscus or portion thereof of a patient may be conducted (426), followed by registration of aspects of image information relative to each other based upon anatomic geometry and details of images; registered image data pertinent to the tissue structures of interest in the patient may be produced (428). Interventional preparation may be conducted to fix an operating table and an intervention platform relative to global coordinate system and relative to each other; placement of patient upon operating table with orientation and access selected to facilitate imaging and intervention may be conducted (430). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (432). With interventional transducer head registered relative to the global coordinate system, imaging of the subject anatomy may be initiated to capture adequate information to register to preoperative volume of registered image data pertinent to the location of the targeted meniscus or portion thereof (434). The interventional team may conduct boiling histotripsy intervention of at least a portion of the targeted meniscus or portion thereof using interventional transducer head as registered to anatomy via continued real-time imaging which remains registered to the known preoperative and intraoperative image data, such as via the use of one or more electromechanical manipulators (436).

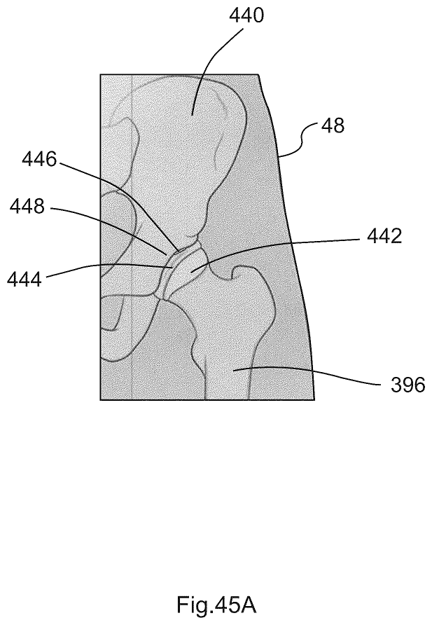

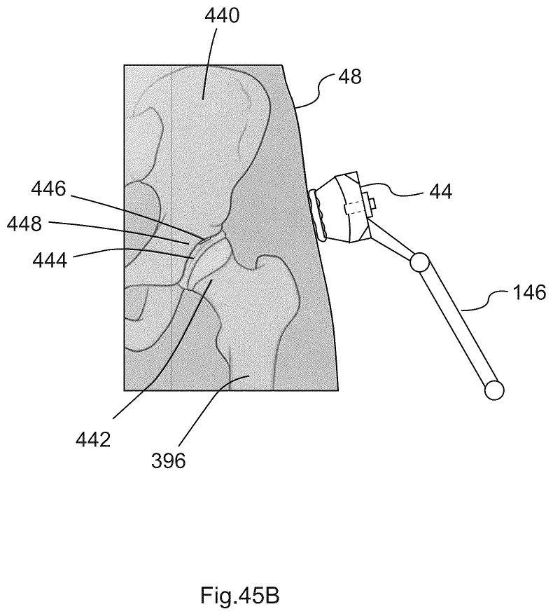

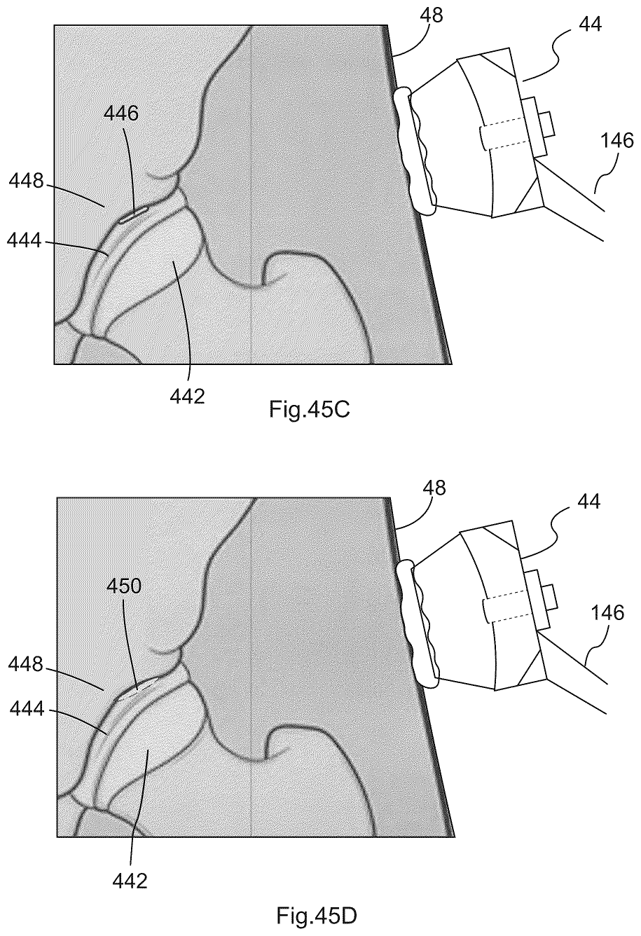

Referring to FIG. 45A, various structures of the hip of a patient (18) are shown, including the femur (396), femoral head (442), pelvis (440), acetabulum (448), hip joint labrum (444), and an injured portion (446) of a hip joint labrum. Injuries to various structures of the hip, such as to a hip joint labrum (444), may cause significant problems, and utilizing conventional surgical approaches can be very invasive. As shown in FIGS. 45B,45C, and 45D, an interventional BH HIFU transducer head (44) may be positioned and/or oriented with image-guided precision, such as via an image-guided electromechanical manipulator (146), to emulsify and/or atomize such aspects of structures such as frayed or damaged portions (446) of a targeted hip joint labrum (444) or portion thereof, followed by potential image-guided aspiration and/or injection, leaving a reduced injury (450) at the targeted location.

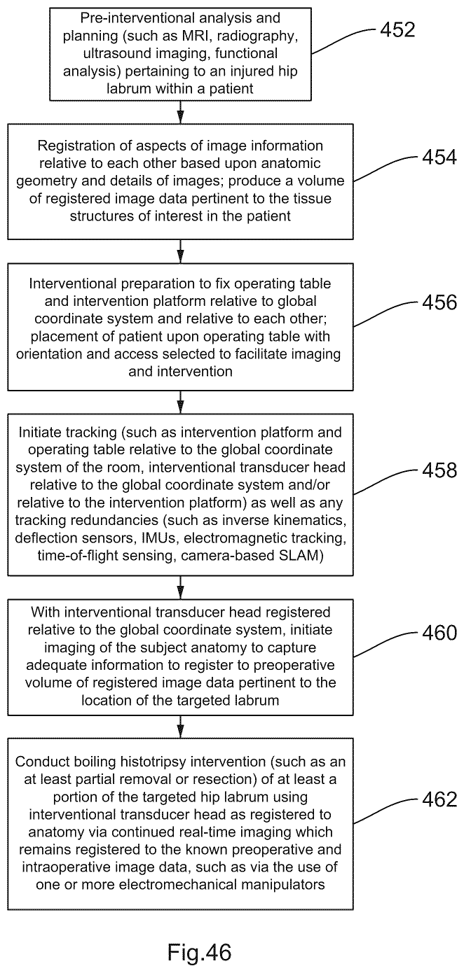

Referring to FIG. 46, pre-interventional analysis and planning (such as MRI, radiography, ultrasound imaging, functional analysis) pertaining to an aspect of at least one injured hip joint labrum within a patient may be conducted (452), followed by registration of aspects of image information relative to each other based upon anatomic geometry and details of images; registered image data pertinent to the tissue structures of interest in the patient may be produced (454). Interventional preparation may be conducted to fix an operating table and an intervention platform relative to global coordinate system and relative to each other; placement of patient upon operating table with orientation and access selected to facilitate imaging and intervention may be conducted (456). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (458). With interventional transducer head registered relative to the global coordinate system, imaging of the subject anatomy may be initiated to capture adequate information to register to preoperative volume of registered image data pertinent to the location of the targeted hip joint labrum (460). The interventional team may conduct boiling histotripsy intervention of at least a portion of the targeted hip joint labrum using interventional transducer head as registered to anatomy via continued real-time imaging which remains registered to the known preoperative and intraoperative image data, such as via the use of one or more electromechanical manipulators (462).

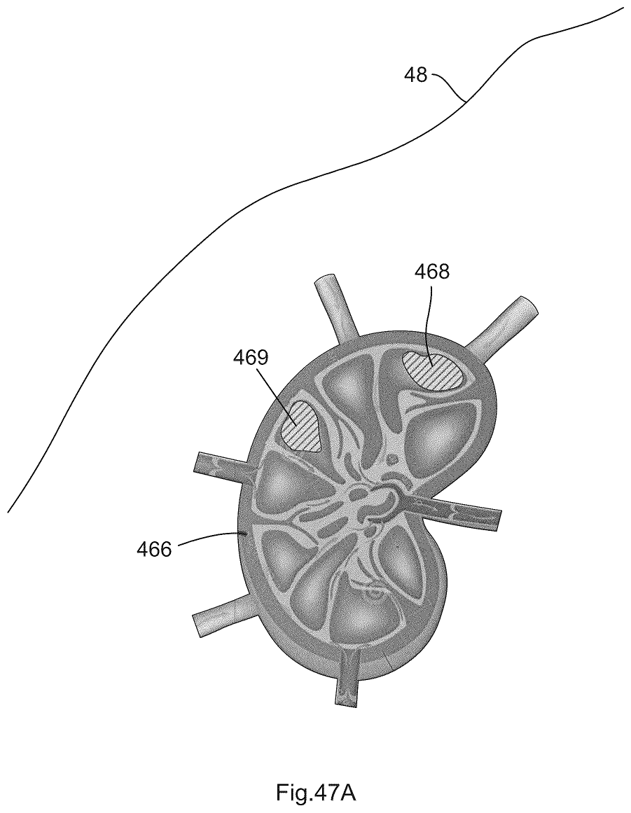

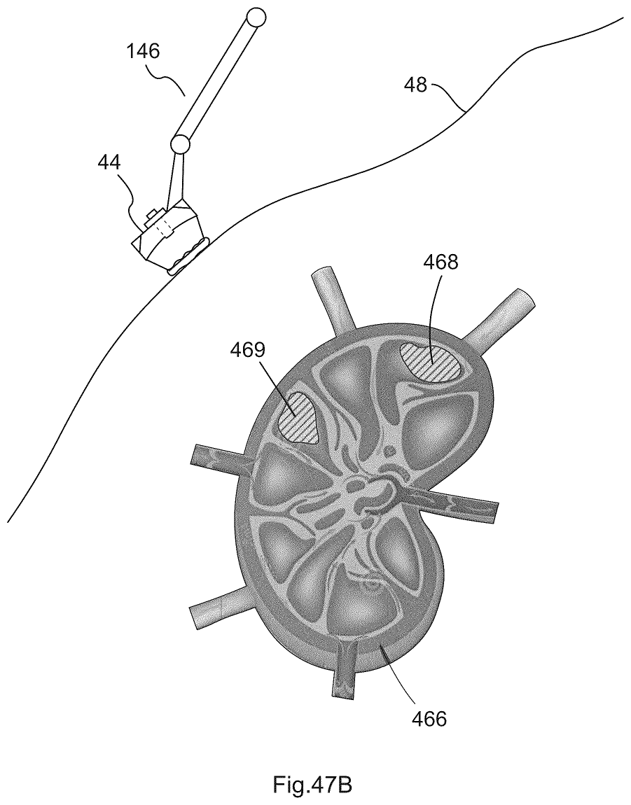

Referring to FIG. 47A, various aspects of a lymph node (466) are shown, including at least two lesions of cancerous lymphoma cells (468, 469). Conventional surgical approaches to address such cancerous lesions can be very invasive. As shown in FIG. 47B, an interventional BH HIFU transducer head (44) may be positioned and/or oriented with image-guided precision, such as via an image-guided electromechanical manipulator (146), to emulsify and/or atomize lymphoma cell lesions, followed by potential image-guided aspiration and/or injection.

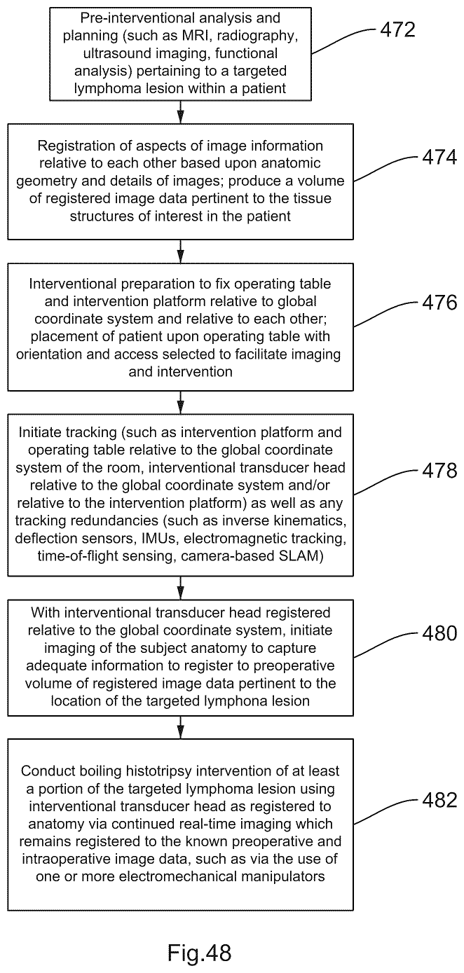

Referring to FIG. 48, pre-interventional analysis and planning (such as MRI, radiography, ultrasound imaging, functional analysis) pertaining to an aspect of at least one lymphoma cell lesion within a patient may be conducted (472), followed by registration of aspects of image information relative to each other based upon anatomic geometry and details of images; registered image data pertinent to the tissue structures of interest in the patient may be produced (474). Interventional preparation may be conducted to fix an operating table and an intervention platform relative to global coordinate system and relative to each other; placement of patient upon operating table with orientation and access selected to facilitate imaging and intervention may be conducted (476). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (478). With interventional transducer head registered relative to the global coordinate system, imaging of the subject anatomy may be initiated to capture adequate information to register to preoperative volume of registered image data pertinent to the location of the targeted lymphoma (480). The interventional team may conduct boiling histotripsy intervention of at least a portion of the targeted lymphoma using interventional transducer head as registered to anatomy via continued real-time imaging which remains registered to the known preoperative and intraoperative image data, such as via the use of one or more electromechanical manipulators (482).

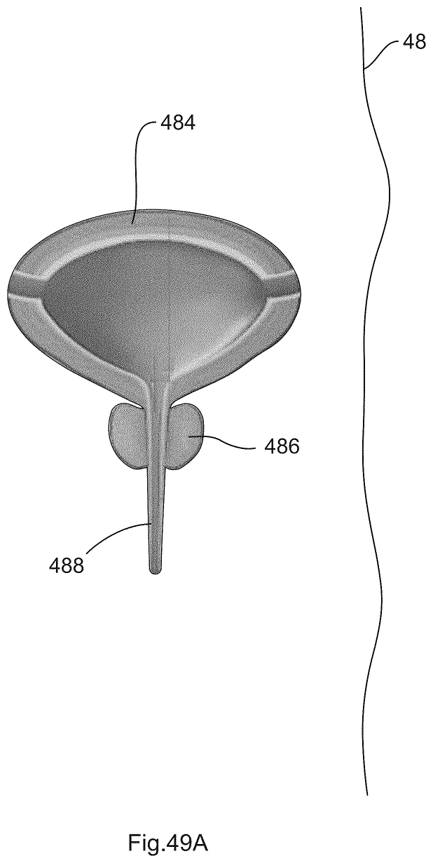

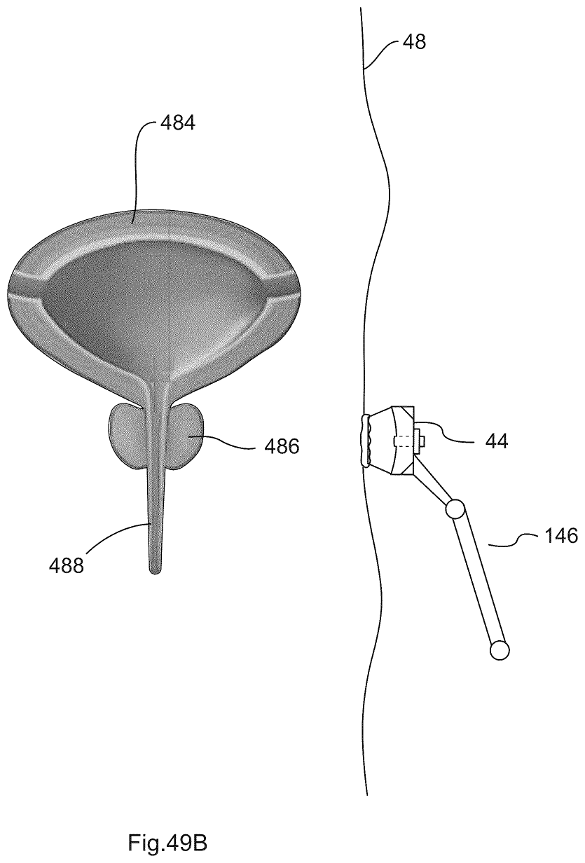

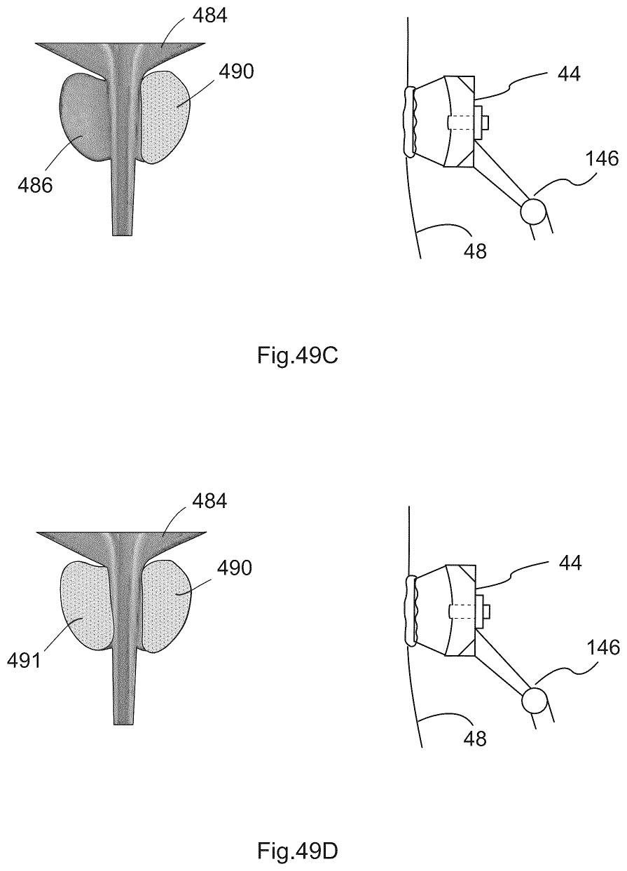

Referring to FIG. 49A, a male human bladder (484), prostate gland (486), and urethra (488) are shown. Conventional surgical approaches to remove portions or all of the prostate gland, such as via direct open surgery or trans-urethral-radical-prostatectomy (or “TURP”) can be very invasive and have varying levels of efficacy and complication. As shown in FIGS. 49B-49D, an interventional BH HIFU transducer head (44) may be positioned and/or oriented with image-guided precision, such as via an image-guided electromechanical manipulator (146), to emulsify and/or atomize (490, 491) portions or all of a prostate gland (486), followed by potential image-guided aspiration and/or injection.

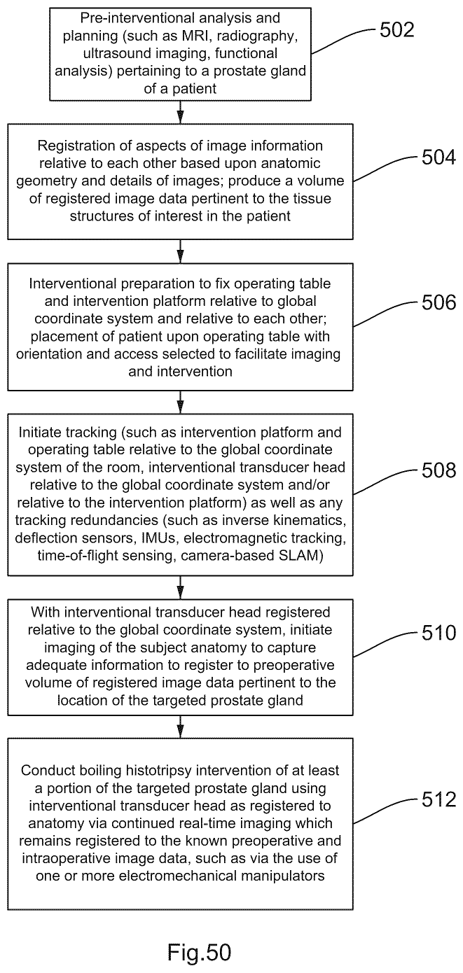

Referring to FIG. 50, pre-interventional analysis and planning (such as MRI, radiography, ultrasound imaging, functional analysis) pertaining to an aspect of a prostate gland within a patient may be conducted (502), followed by registration of aspects of image information relative to each other based upon anatomic geometry and details of images; registered image data pertinent to the tissue structures of interest in the patient may be produced (504). Interventional preparation may be conducted to fix an operating table and an intervention platform relative to global coordinate system and relative to each other; placement of patient upon operating table with orientation and access selected to facilitate imaging and intervention may be conducted (506). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (508). With interventional transducer head registered relative to the global coordinate system, imaging of the subject anatomy may be initiated to capture adequate information to register to preoperative volume of registered image data pertinent to the location of the targeted prostate gland or portion thereof (510). The interventional team may conduct boiling histotripsy intervention of at least a portion of the targeted prostate gland or portion thereof using interventional transducer head as registered to anatomy via continued real-time imaging which remains registered to the known preoperative and intraoperative image data, such as via the use of one or more electromechanical manipulators (512).

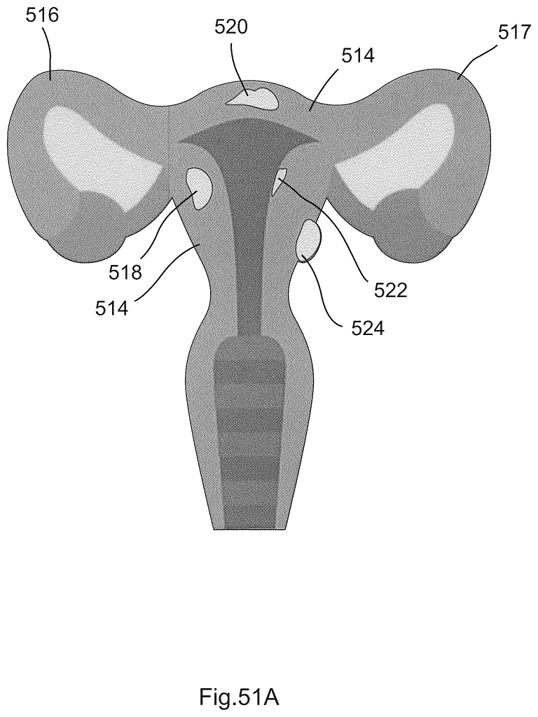

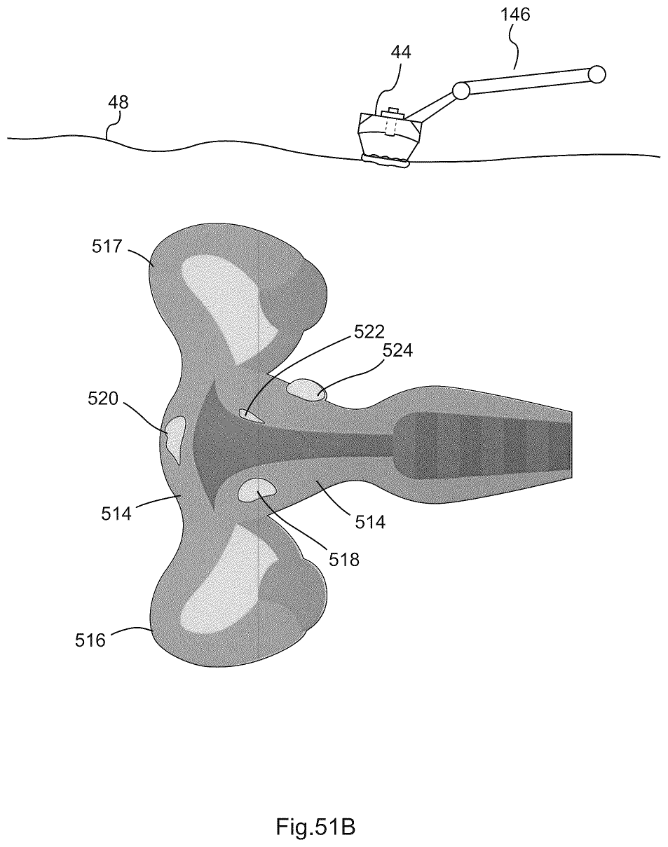

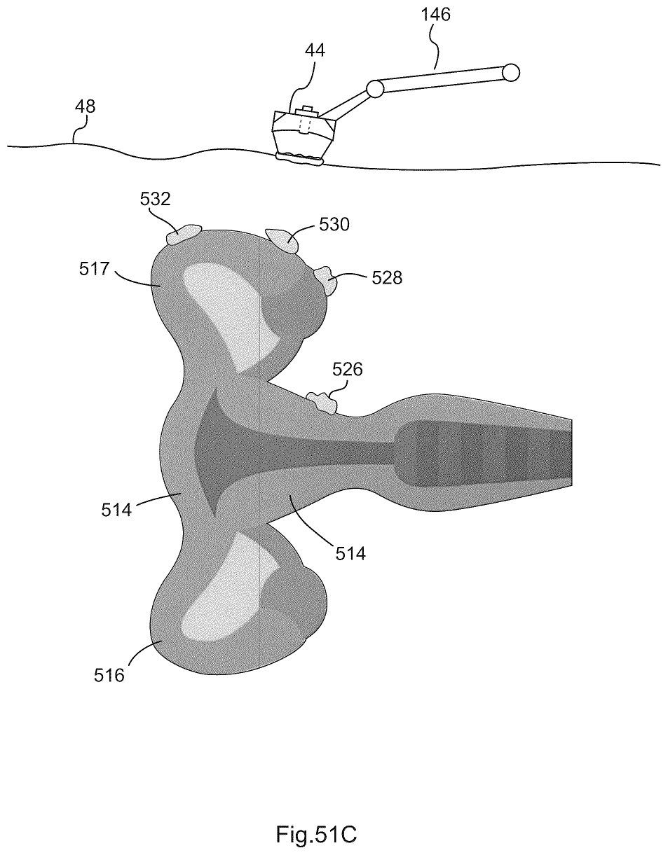

Referring to FIG. 51A, a female human uterus (514) and fallopian tubes (516, 517) are shown, along with a group of fibroid tumors (518, 520, 522, 524) positioned in various locations and tissue depths relative to the involved uterus (514). Conventional surgical approaches to remove portions or all of fibroid tumors, such as via direct open surgery, can be very invasive and have varying levels of efficacy and complication. As shown in FIG. 51B, an interventional BH HIFU transducer head (44) may be positioned and/or oriented with image-guided precision, such as via an image-guided electromechanical manipulator (146), to emulsify and/or atomize portions or all of targeted fibroid tumors (518, 520, 522, 524) or portions thereof, followed by potential image-guided aspiration and/or injection.

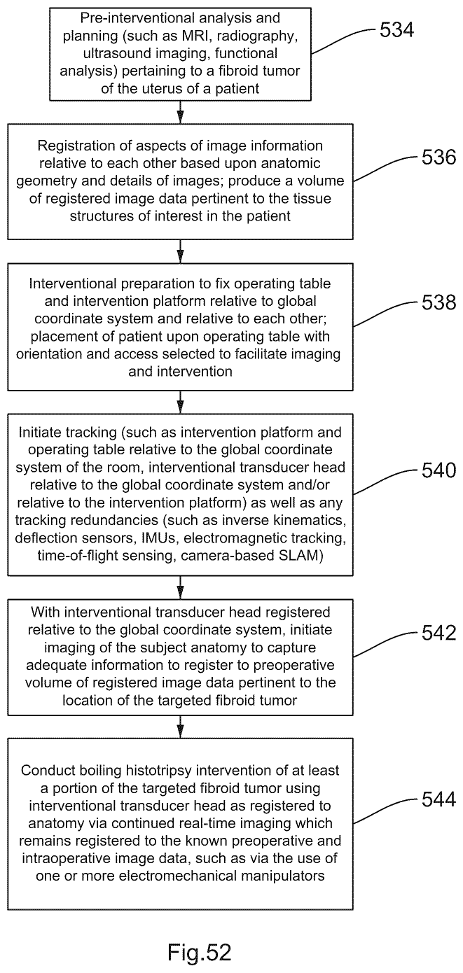

Referring to FIG. 52, pre-interventional analysis and planning (such as MRI, radiography, ultrasound imaging, functional analysis) pertaining to an aspect of a fibroid tumor within a patient may be conducted (534), followed by registration of aspects of image information relative to each other based upon anatomic geometry and details of images; registered image data pertinent to the tissue structures of interest in the patient may be produced (536). Interventional preparation may be conducted to fix an operating table and an intervention platform relative to global coordinate system and relative to each other; placement of patient upon operating table with orientation and access selected to facilitate imaging and intervention may be conducted (538). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (540). With interventional transducer head registered relative to the global coordinate system, imaging of the subject anatomy may be initiated to capture adequate information to register to preoperative volume of registered image data pertinent to the location of the targeted fibroid tumor or portion thereof (542). The interventional team may conduct boiling histotripsy intervention of at least a portion of the targeted fibroid tumor or portion thereof using interventional transducer head as registered to anatomy via continued real-time imaging which remains registered to the known preoperative and intraoperative image data, such as via the use of one or more electromechanical manipulators (544).

Referring to FIG. 51C, a female human uterus (514) and fallopian tubes (516, 517) are shown, along with a group of endometrium lesions (526, 528, 530, 532) positioned in various locations and couplings relative to the involved uterus (514) and fallopian tube (517). Conventional surgical approaches to remove portions or all of endometrium lesions, such as via direct open surgery, can be very invasive and have varying levels of efficacy and complication. As shown in FIG. 51C, an interventional BH HIFU transducer head (44) may be positioned and/or oriented with image-guided precision, such as via an image-guided electromechanical manipulator (146), to emulsify and/or atomize portions or all of targeted emdometrium lesions (526, 528, 530, 532) or portions thereof, followed by potential image-guided aspiration and/or injection.

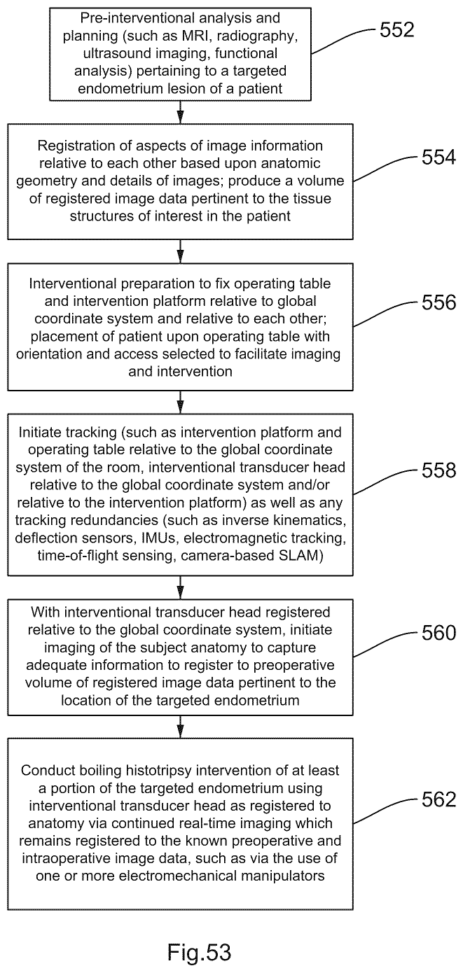

Referring to FIG. 53, pre-interventional analysis and planning (such as MRI, radiography, ultrasound imaging, functional analysis) pertaining to an aspect of an endometrium lesion within a patient may be conducted (552), followed by registration of aspects of image information relative to each other based upon anatomic geometry and details of images; registered image data pertinent to the tissue structures of interest in the patient may be produced (554). Interventional preparation may be conducted to fix an operating table and an intervention platform relative to global coordinate system and relative to each other; placement of patient upon operating table with orientation and access selected to facilitate imaging and intervention may be conducted (556). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (558). With interventional transducer head registered relative to the global coordinate system, imaging of the subject anatomy may be initiated to capture adequate information to register to preoperative volume of registered image data pertinent to the location of the targeted endometrium lesion or portion thereof (560). The interventional team may conduct boiling histotripsy intervention of at least a portion of the targeted endometrium lesion or portion thereof using interventional transducer head as registered to anatomy via continued real-time imaging which remains registered to the known preoperative and intraoperative image data, such as via the use of one or more electromechanical manipulators (562).

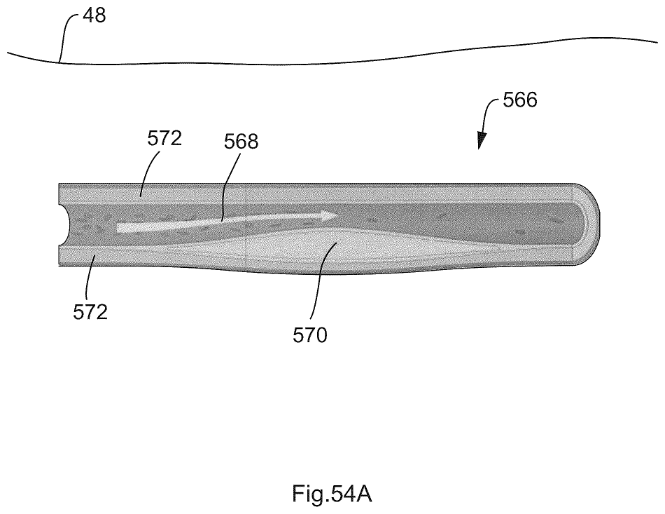

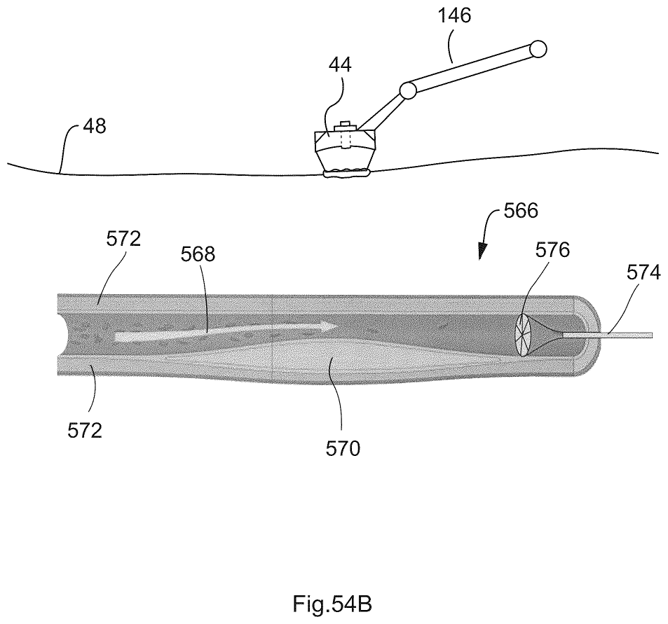

Referring to FIG. 54A, a blood vessel (566), such as a human artery, is shown, comprising a vessel wall (572) which defines a blood flow pathway (568) which is, in the depicted scenario, partially blocked by a plaque structure (570) which has formed. Conventional surgical approaches to remove portions or all of such plaque structures, such as via direct open vascular surgery, can be very invasive and have varying levels of efficacy and complication. As shown in FIG. 54B, an interventional BH HIFU transducer head (44) may be positioned and/or oriented with image-guided precision, such as via an image-guided electromechanical manipulator (146), to emulsify and/or atomize portions or all of targeted plaque structure or portions thereof, followed by potential image-guided aspiration and/or injection. Referring to FIG. 54B, an endovascular device (574), such as a catheter which features a collapsible screen portion (576), and which may be configured to have a defined lumen therethrough to provide a vacuum/suction and/or aspiration (such as those available for capturing clots during neurovascular and other interventions), may be used to assist in capturing plaque material which becomes removable by virtue of the BH HIFU intervention.

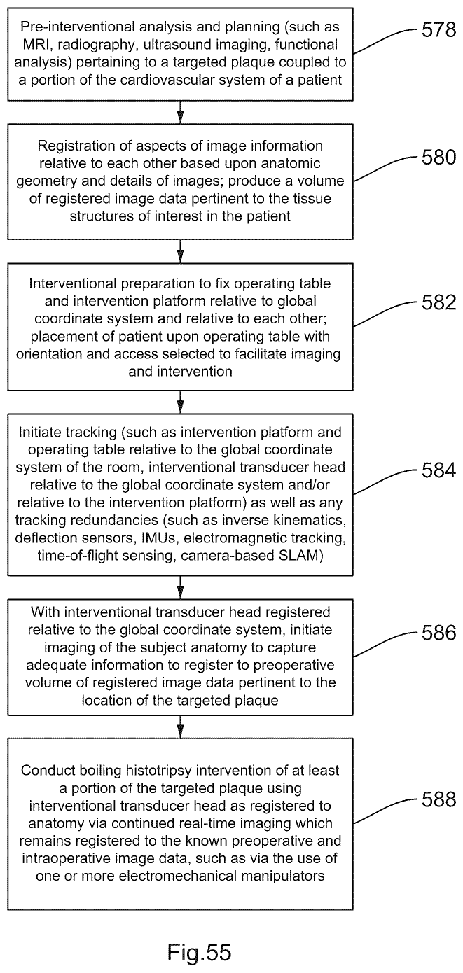

Referring to FIG. 55, pre-interventional analysis and planning (such as MRI, radiography, ultrasound imaging, functional analysis) pertaining to an aspect of a plaque structure within a patient may be conducted (578), followed by registration of aspects of image information relative to each other based upon anatomic geometry and details of images; registered image data pertinent to the tissue structures of interest in the patient may be produced (580). Interventional preparation may be conducted to fix an operating table and an intervention platform relative to global coordinate system and relative to each other; placement of patient upon operating table with orientation and access selected to facilitate imaging and intervention may be conducted (582). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (584). With interventional transducer head registered relative to the global coordinate system, imaging of the subject anatomy may be initiated to capture adequate information to register to preoperative volume of registered image data pertinent to the location of the targeted plaque or portion thereof (586). The interventional team may conduct boiling histotripsy intervention of at least a portion of the targeted plaque or portion thereof using interventional transducer head as registered to anatomy via continued real-time imaging which remains registered to the known preoperative and intraoperative image data, such as via the use of one or more electromechanical manipulators (588).

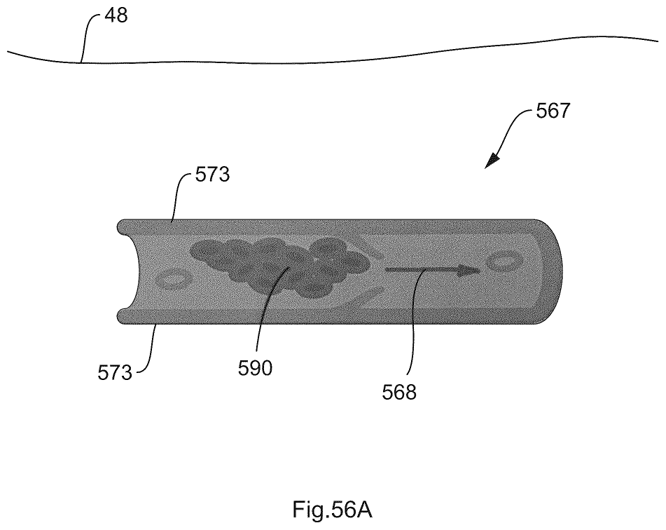

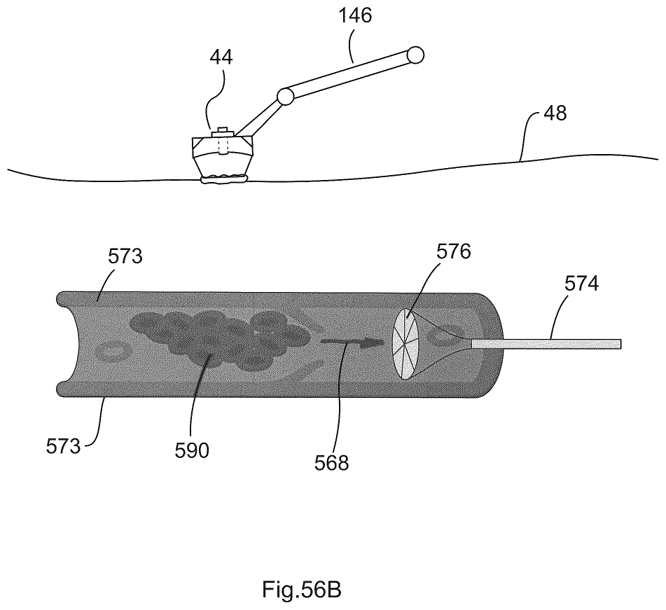

Referring to FIG. 56A, a blood vessel (567), such as a human peripheral vein, is shown, comprising a vessel wall (573) which defines a blood flow pathway (568) which is, in the depicted scenario, partially blocked by a blood clot or embolism structure or mass (590) which has formed. Conventional surgical approaches to remove portions or all of such clot or embolism structures or masses, such as via direct open vascular surgery, can be very invasive and have varying levels of efficacy and complication. As shown in FIG. 56B, an interventional BH HIFU transducer head (44) may be positioned and/or oriented with image-guided precision, such as via an image-guided electromechanical manipulator (146), to emulsify and/or atomize portions or all of targeted clot or embolism structure or portions thereof, followed by potential image-guided aspiration and/or injection. Referring to FIG. 56B, an endovascular device (574), such as a catheter which features a collapsible screen portion (576), and which may be configured to have a defined lumen therethrough to provide a vacuum/suction and/or aspiration (such as those available for capturing clots during neurovascular and other interventions), may be used to assist in capturing plaque material which becomes removable by virtue of the BH HIFU intervention.

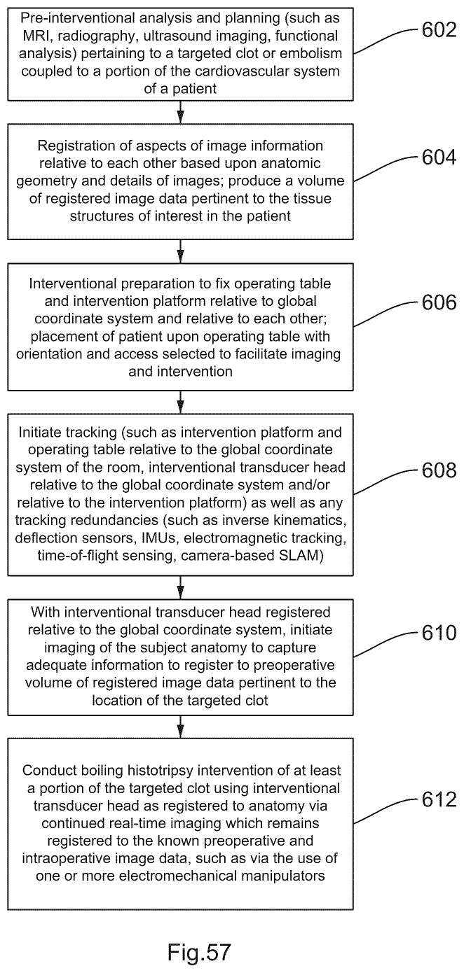

Referring to FIG. 57, pre-interventional analysis and planning (such as MRI, radiography, ultrasound imaging, functional analysis) pertaining to an aspect of a clot or embolism within a patient may be conducted (602), followed by registration of aspects of image information relative to each other based upon anatomic geometry and details of images; registered image data pertinent to the tissue structures of interest in the patient may be produced (604). Interventional preparation may be conducted to fix an operating table and an intervention platform relative to global coordinate system and relative to each other; placement of patient upon operating table with orientation and access selected to facilitate imaging and intervention may be conducted (606). Tracking may be initiated (such as intervention platform and operating table relative to the global coordinate system of the room, interventional transducer head relative to the global coordinate system and/or relative to the intervention platform) as well as any tracking redundancies (such as inverse kinematics, deflection sensors, IMUs, electromagnetic tracking, time-of-flight sensing, camera-based SLAM) (608). With interventional transducer head registered relative to the global coordinate system, imaging of the subject anatomy may be initiated to capture adequate information to register to preoperative volume of registered image data pertinent to the location of the targeted clot or embolism or portion thereof (610). The interventional team may conduct boiling histotripsy intervention of at least a portion of the targeted clot or embolism or portion thereof using interventional transducer head as registered to anatomy via continued real-time imaging which remains registered to the known preoperative and intraoperative image data, such as via the use of one or more electromechanical manipulators (612).

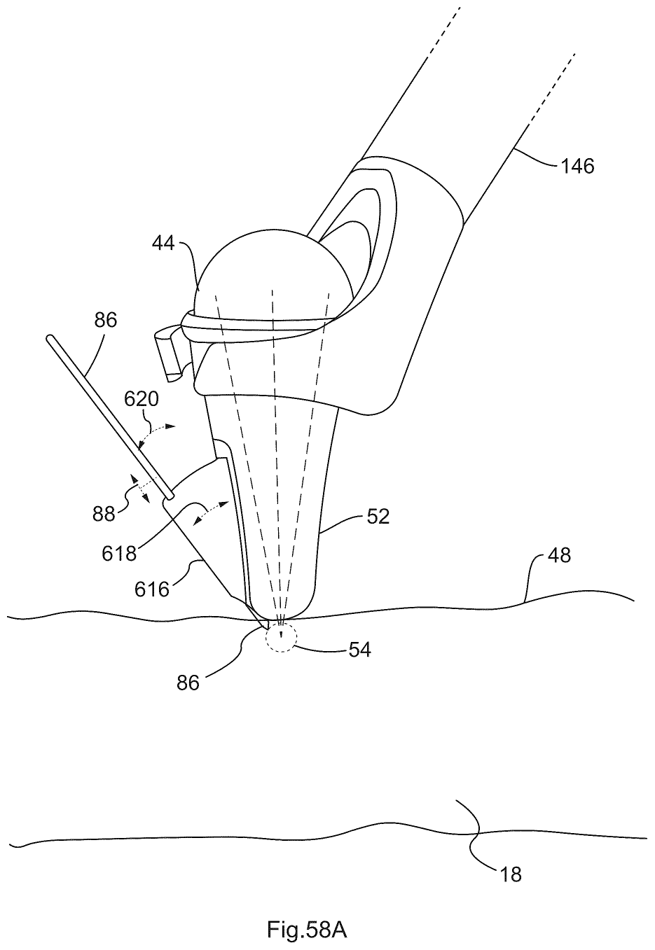

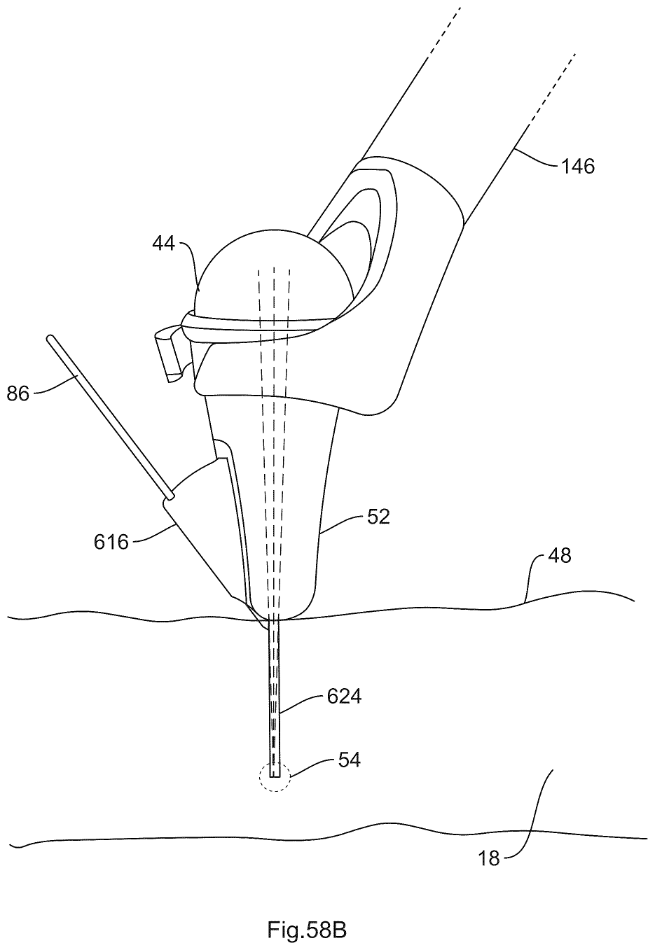

Referring back to FIG. 10D, in various embodiments an additional instrument (86), such as a cannula or needle, may be inserted (88) or retracted along a known orientation relative to the transducer (44) to assist with various aspects of a medical procedure, such as for controlled and image-guided injection and/or aspiration. Referring to FIGS. 58A and 58B, partial orthogonal views of related embodiments are illustrated. Referring to FIG. 58A, a transducer (44) may be operatively coupled to an arm or mounting structure such as a robotic manipulator (146) such that the interface (52) is positioned and oriented to facilitate ultrasound emission at the focal point (54) while a distal portion of the additional instrument (86) is directed to the same focal point (54). A movable housing (616) for the additional instrument (86) may be configured to controllably reorient (618) relative to the interface (52) housing, thereby controllably reorienting (618) the additional instrument (86) relative to the transducer (44), such as via an electromechanical subsystem controllable by an operator, which may also be configured to control precision insertion/retraction (88) of the additional instrument (86). Referring to FIG. 58B, a variation similar to that of FIG. 58A is illustrated, also featuring a secondary additional instrument (624), which may be controllably insertable and retractable (such as by electromechanical actuation) relative to the interface (52) housing, and which also may comprise an elongate instrument such as a needle or cannula, which may be utilized to physically address a remote focal point (54) for applied ultrasonic radiation, as shown in FIG. 58B. This elongate instrument (624) may also be utilized to assist in precision transmission of treatment and/or imaging radiation en route to the focal point (54), and may comprise a waveguide or refractive device configured to assist in transmitting and/or focusing transmitted radiation, such as ultrasonic radiation. Instrumentation utilized with the subject systems may be coated with, or may comprise, materials which are selected to be luminescent or reflective relative to the associated applied ultrasonic radiation, and may also be configured to emit radiation, such as various wavelengths of light or other radiation, to assist with identification and visualization during image-guided procedures.

In various embodiments, low-intensity pulsed ultrasound (which also may be known as “LIPUS”) may be transmitted from the transducer and utilized to assist in precision directed stimulation and/or healing of collagenous or other soft tissue, or calcified tissue such as fracture locations. Such LIPUS configurations may also be utilized for cosmesis purposes, such as image-guided non-invasive reshaping of one or more tissue structures, such as to correct or adjust the shape of a disfiguring tissue structure. Image-guided ultrasonic radiation from subject configurations may also be utilized to monitor curing of an implantable compound (such as such as one comprising a thermosetting resin, which may undergo a molecular “crosslinking” process in “curing” during which it changes irreversibly from being at least a portion of viscous liquid to a more rigid and highly cross-linked polymer solid) which may be injected into cavity which has been newly formed, for example. Such variations may be configured to detect sound velocity and attenuation, which are very sensitive to changes in the viscoelastic characteristics of the curing resin, since the velocity is related to the resin storage modulus and density, while the attenuation is related to the energy dissipation and scattering in the curing resin. Image-guided ultrasonic radiation from subject configurations may also be utilized to create additional local micromotion to enhance or facilitate curing or tissue healing.

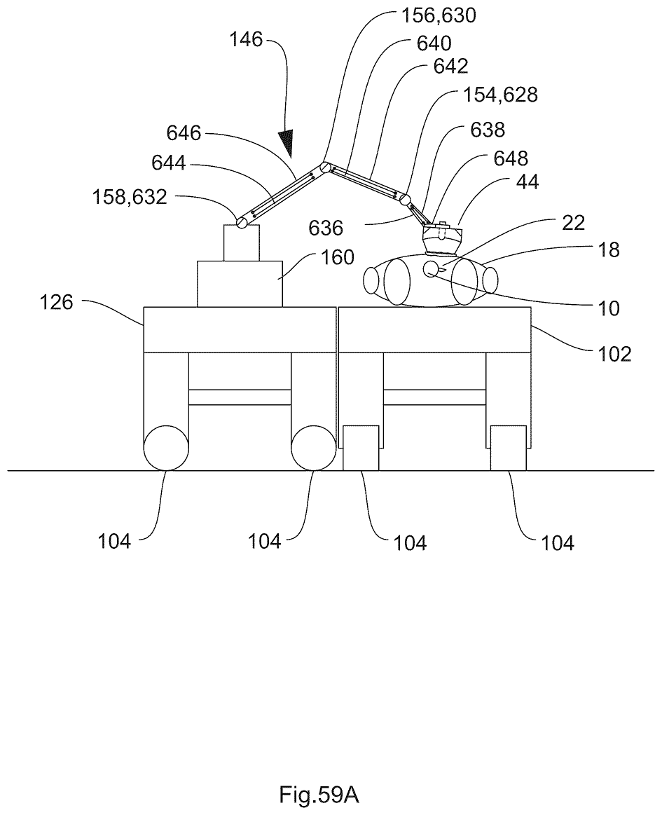

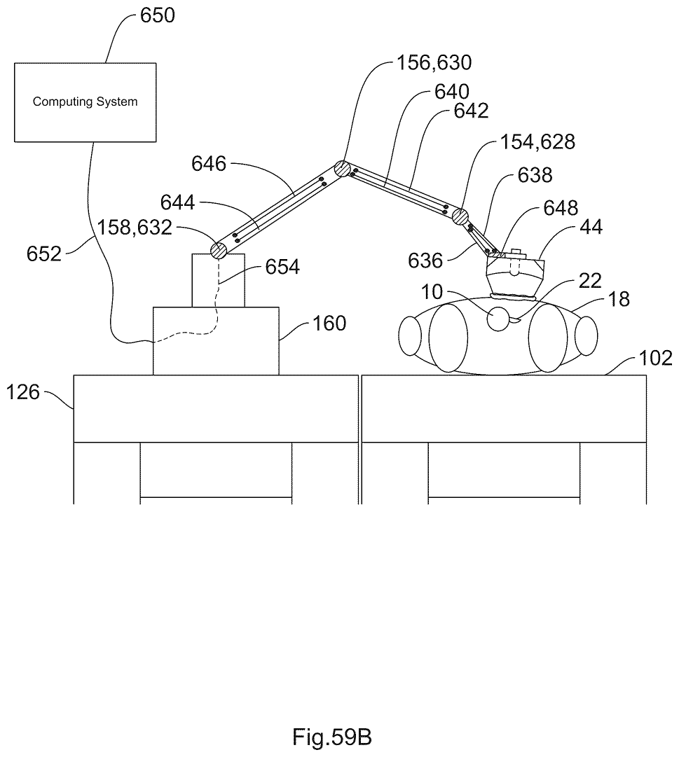

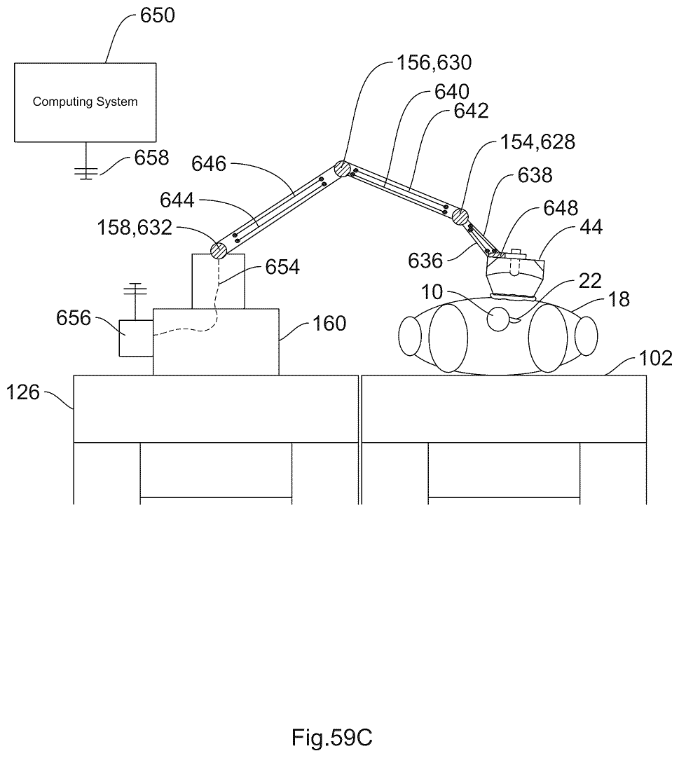

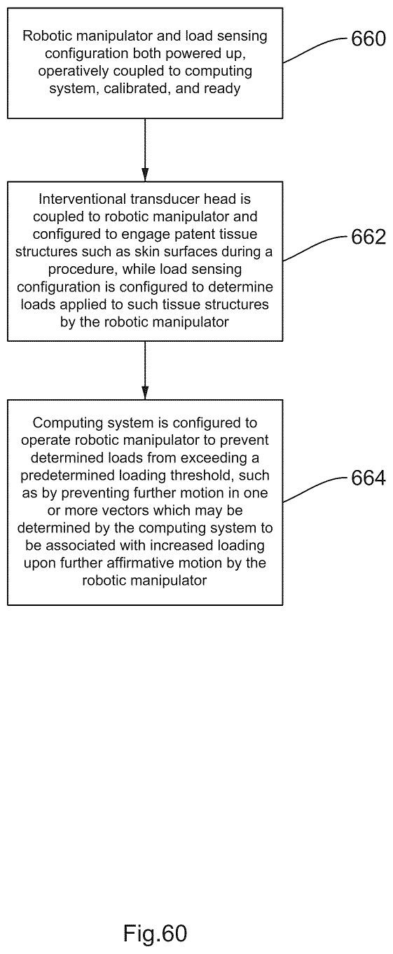

Referring to FIGS. 59A-61, various configurations pertaining to load control with a transducer head (44) are illustrated. Referring to FIG. 59A, an embodiment similar to that of FIG. 10A is illustrated with a robotic manipulator (146) assembly coupled to a transducer head (44) which may be positioned adjacent a patient (18) for diagnostic and/or interventional purposes. The manipulator assembly (146) may be configured to assist in determination of loads applied by the transducer head (44) to the patient (18). For example, in one embodiment, joint encoders at the joints (154, 156, 158, for example) and kinematic relationships may be utilized with so-called “inverse kinematics” techniques, along with an intercoupled computing system, to estimate loads applied by the manipulator (146) and intercoupled transducer head (44) to the patient (18). The joints (154, 156, 158, for example) also may be fitted with load sensors, such as optics based load sensors, or torque sensors, such as those available from ATI Industrial Automation (a Novanta Company). Further, various lengths of the subject assembly may be fitted with elongate sensors (636, 638, 640, 642, 644, 646), such as strain gauges, which may be based upon configurations such as those utilizing detection of elongation of wire leads, or detection of elongation of optical fibers (as in the case of a fiber-Bragg deflection sensing fiber configuration), for example. Multiple configurations selected from those including inverse kinematics, joint encoders, strain gauges, joint torque or load sensing, and/or current monitoring, may be utilized to have redundancy of sensing, and may be utilized in a Kalman filter type of configuration wherein uncorrelated errors of redundant sensing systems may be advantageous. Referring to FIG. 59B, such sensors may be operatively coupled, such as via wired lead (652; or via wireless connectivity, such as between two or more wireless transceivers 656, 658, as shown in FIG. 59C), to a computing system which also may be operatively coupled to the motor controller subsystem of the robotic manipulator (146). Referring to FIG. 60, a robotic manipulator and load sensing configuration both may be powered up, operatively coupled to computing system, calibrated, and ready (660). An interventional transducer head may be coupled to robotic manipulator and configured to engage patent tissue structures such as skin surfaces during a procedure, while load sensing configuration is configured to determine loads applied to such tissue structures by the robotic manipulator (662). A computing system may be configured to operate robotic manipulator to prevent determined loads from exceeding a predetermined loading threshold, such as by preventing further motion in one or more vectors which may be determined by the computing system to be associated with increased loading upon further affirmative motion by the robotic manipulator (664). For example, in one embodiment, the computing system may be configured to maintain an interfacial load between a HIFU transducer and a portion of the patient's tissue that is below a predetermined maximum load; in another embodiment, the computing system may be configured to maintain the interfacial load above a predetermined minimum and below a predetermined maximum during a period of treatment of the patient; in another embodiment the computing system is configured to maintain a relative orientation (such as a configuration whereby the electromechanical support assembly may be configured to dynamically adjust position and/or orient to have the HIFU treatment transducer follow the surface orientation of the subject tissue surface being contacted on the patient; in other words, an automatic terrain or surface following configuration) between the HIFU treatment transducer and a most immediately adjacent portion of the patient's body while also maintaining the interfacial load above a predetermined minimum and below a predetermined maximum during a period of treatment of the patient; in another embodiment the computing system may be configured to facilitate a repositioning of the HIFU treatment transducer relative to the patient while also maintaining the relative orientation between the HIFU treatment transducer and most immediately adjacent portion of the patient's body, as well as also maintaining the interfacial load above a predetermined minimum and below a predetermined maximum during a period of treatment of the patient (in other words, an automatic terrain or surface following configuration under interfacial load control), such that the transducer may be moved manually, such as in response to predetermined or manual commands, such as at an operator interface operably coupled to the computing system, or automatically relative to the coupled tissue in a direction orthogonal to the contact vector, while maintaining orientation and loading at the contact vector (for example, the transducer may be moved manually or automatically in an X-axis or Y-axis direction while the system maintains contact, loading, and orientation in a Z axis direction).

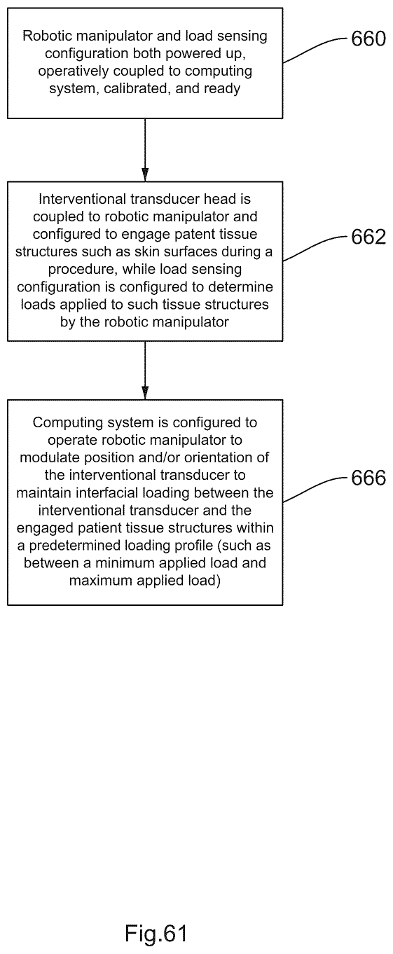

Referring to FIG. 61, a robotic manipulator and load sensing configuration both may be powered up, operatively coupled to computing system, calibrated, and ready (660). An interventional transducer head may be coupled to robotic manipulator and configured to engage patent tissue structures such as skin surfaces during a procedure, while load sensing configuration is configured to determine loads applied to such tissue structures by the robotic manipulator (662). A computing system may be configured to operate robotic manipulator to modulate position and/or orientation of the interventional transducer to maintain interfacial loading between the interventional transducer and the engaged patient tissue structures within a predetermined loading profile (such as between a minimum applied load and maximum applied load) (666).

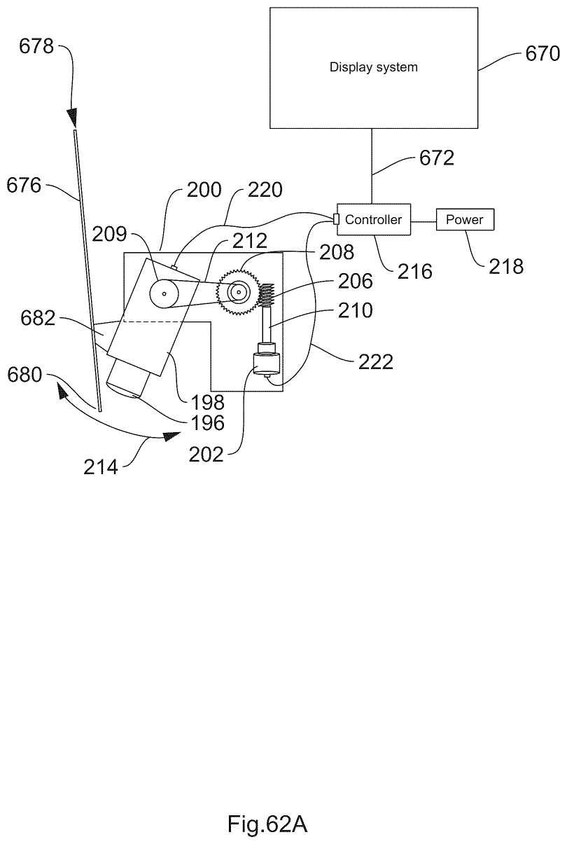

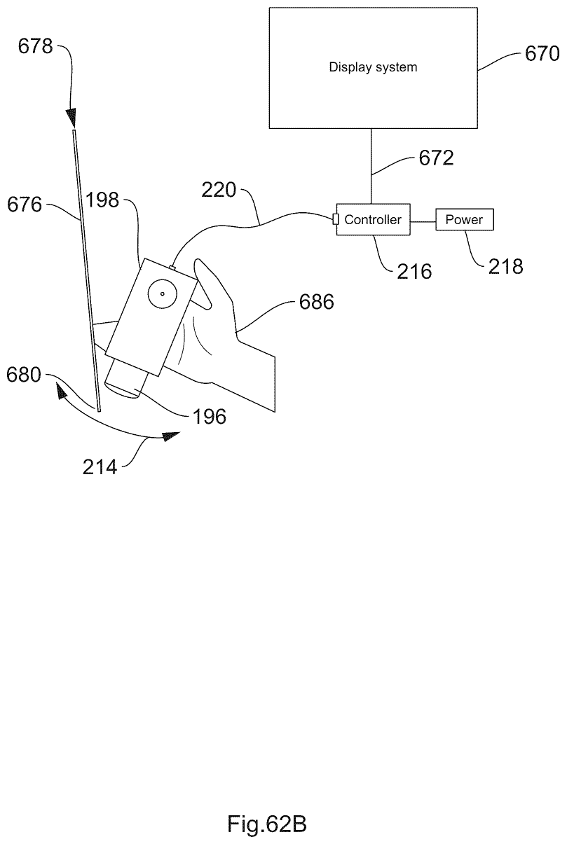

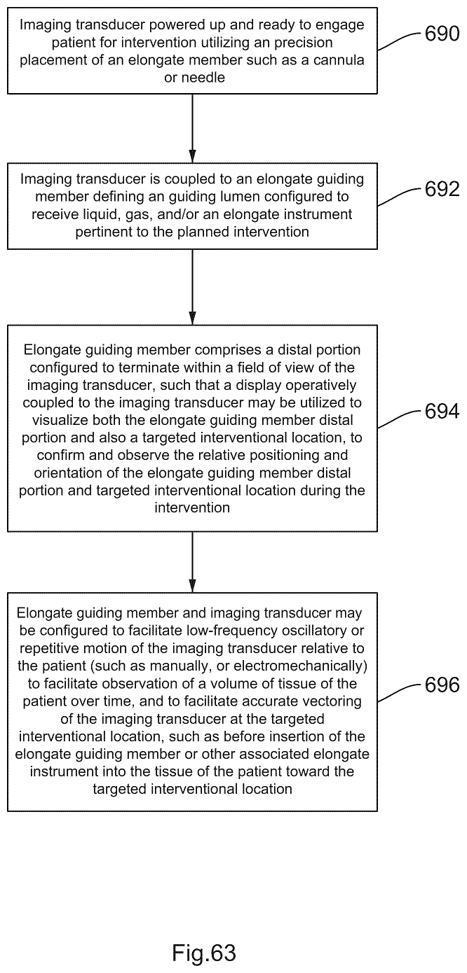

Referring to FIGS. 62A-63, a transducer (196) may be manually (as with the intercoupled hand 686 of FIG. 62B) or electromechanically (as with the intercoupled motor/drive configuration, similar to that shown in FIGS. 16A and 16B above, of FIG. 62A). A mounting bracket (682) may be configured to couple an elongate guide member (676) to the housing (198) of the transducer (196) such that a lumen (678) defined through the elongate guide member (676) may terminate at a distal end (680) of the elongate guide member (676) in a position configured to be within the field of view of the transducer (196). In other words, the transducer may be utilized to visualize, such as via ultrasonic imaging, a tissue structure or location of interest, along with a needle or other member inserted through the elongate guide member (676) to a position at or adjacent to that tissue structure or location of interest. Such visualization may be enhanced through manual or electromechanical motion, such as cyclical motion, of the transducer (196) to capture additional “slices” of image data pertinent to the location of interest. For example, referring to FIG. 63, an imaging transducer may be powered up and ready to engage patient for intervention utilizing an precision placement of an elongate member such as a cannula or needle (690). The imaging transducer may be coupled to an elongate guiding member defining an guiding lumen configured to receive liquid, gas, and/or an elongate instrument pertinent to the planned intervention (692). The elongate guiding member may comprise a distal portion configured to terminate within a field of view of the imaging transducer, such that a display operatively coupled to the imaging transducer may be utilized to visualize both the elongate guiding member distal portion and also a targeted interventional location, to confirm and observe the relative positioning and orientation of the elongate guiding member distal portion and targeted interventional location during the intervention (694). The elongate guiding member and imaging transducer may be configured to facilitate low-frequency oscillatory or repetitive motion of the imaging transducer relative to the patient (such as manually, or electromechanically) to facilitate observation of a volume of tissue of the patient over time, and to facilitate accurate vectoring of the imaging transducer at the targeted interventional location, such as before insertion of the elongate guiding member or other associated elongate instrument into the tissue of the patient toward the targeted interventional location (696).

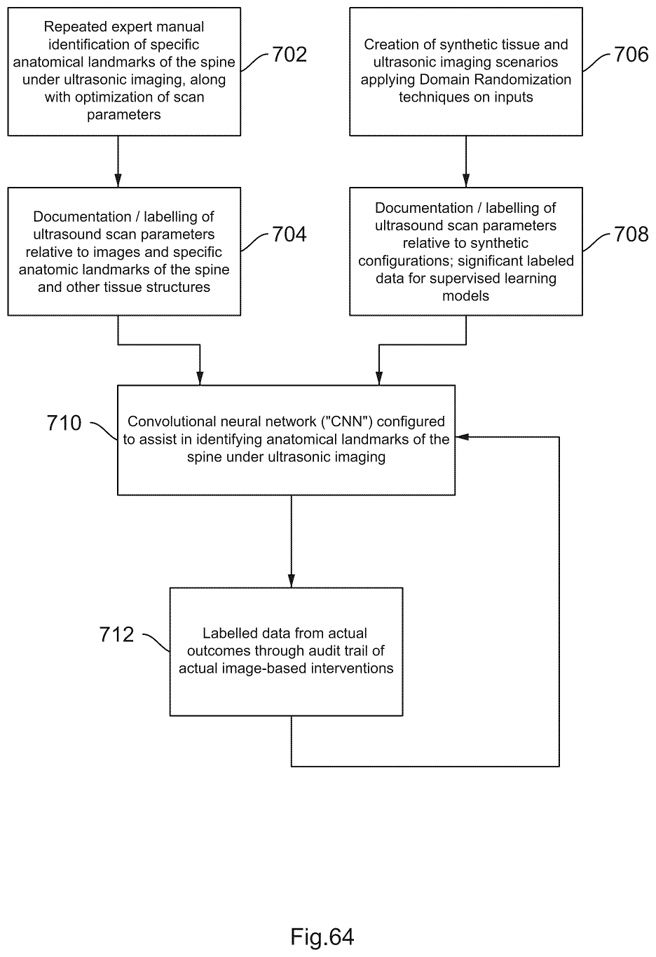

Referring to FIG. 64, supervised learning techniques may be utilized to train a convolutional neural network (710; “CNN”) to assist in recognizing anatomic landmarks from image data, such as ultrasonic image data, such that repeated expert manual identification (i.e., labelling for supervised learning techniques) of specific anatomical landmarks of the spine under ultrasonic imaging, for example, along with optimization of scan parameters (702), may be utilized for training-to assist the system in becoming more and more proficient at automatically identifying tissue structures and landmarks thereof in a given patient scenario without constant supervision of an expert. Such documentation/labelling of the ultrasound scan parameters relative to images, and specific anatomic landmarks of the spine and other tissue structures (704), may be utilized to train the CNN (710). Further, data from actual outcomes may be labelled (712) to also assist in further training the CNN (710). To assist in scaling the training beyond these fairly manual means of having an expert label data for a supervised learning configuration, one or more synthetic data environments may be created and utilized to produce synthetic images from various perspectives with various known/labelled landmarks (706), and this synthetic labeled data also may be utilized to train (708) the CNN as shown in FIG. 64.

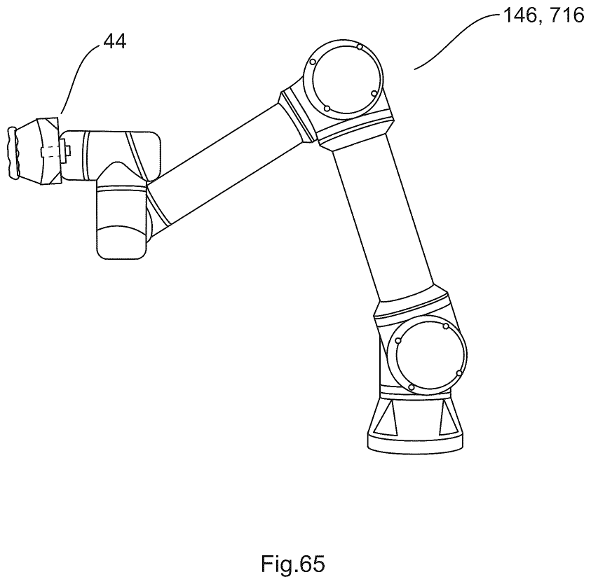

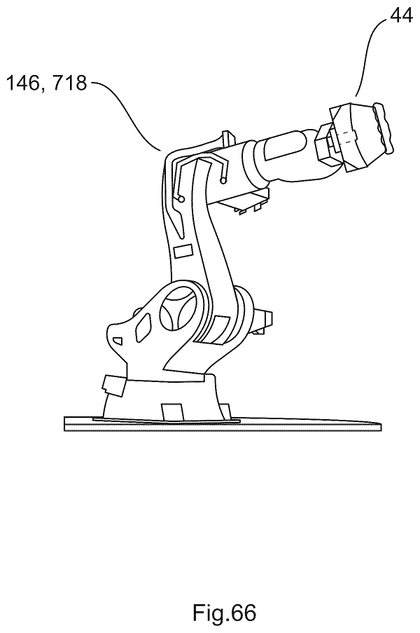

Referring to FIGS. 65 and 66, various off-the-shelf robotic manipulator (146) configurations, such as those available from Universal Robotics A/S (716), or Kuka Robotics Corporation (718), may be utilized to couple with the subject transducer head configurations (44).

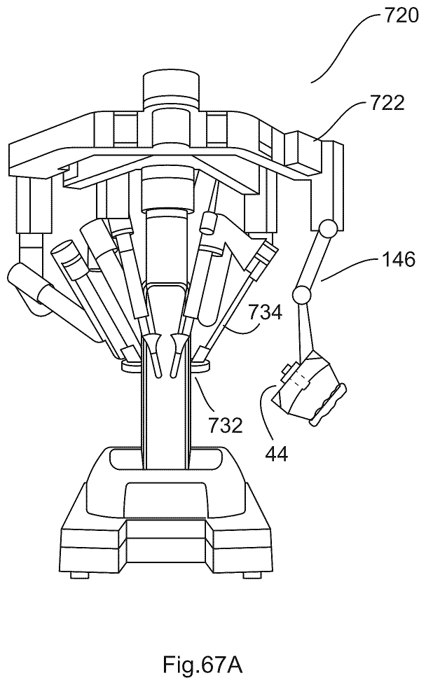

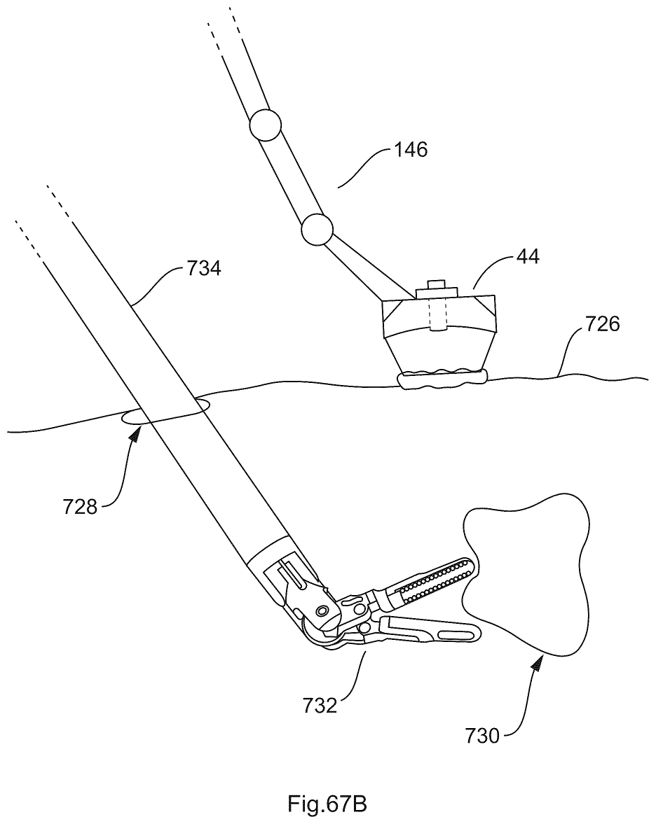

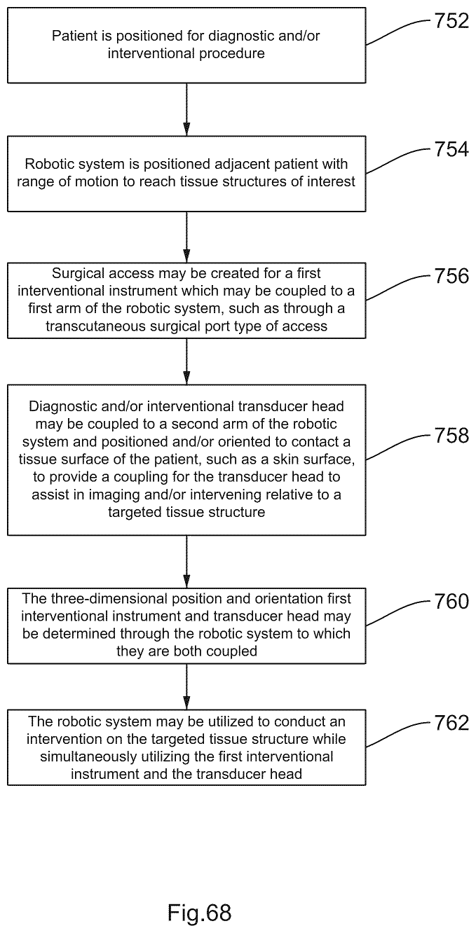

Referring to FIG. 67A, a transducer head (44) and intercoupled robotic manipulator (146) may be coupled to a portion of (such as an arm 722 of) a surgical robotics system such as those sold under the tradename DaVinci (RTM) by Intuitive Surgical, Corporation, and configured to be utilized separately, or in coordination with, one or more of the other instruments (734, 732) comprising such system. The surgical robotics system may comprise a central base from which a plurality of articulated (such as by electromechanical movable joints, position, orientation, and load sensing and control configurations as described above, and the like) robotic arms are extendable, each of which may be coupled to a surgical and/or interventional instrument or end effector, or an interventional HIFU configuration, as shown in FIG. 67A. Thus such a configuration may be utilized to conduct therapeutic and/or diagnostic procedures as described above in reference to the various system configurations. For example, referring to FIG. 67B, an elongate instrument member or shaft (734) coupled to a distal end effector (732) may be at least partially inserted into a patient, such as through a port-access type of surgical access point (728), to facilitate intervention of a targeted tissue structure (730), while coordinated positioning and/or orientation of a manipulator (146) and intercoupled transducer head (44) positioned against a surface of the patient (such as the skin 726) may be utilized to visualize the targeted tissue structure and interventional tool (732, 734) during operation. For example, referring to FIG. 68, a patient may be positioned for diagnostic and/or interventional procedure (752). A robotic system may be positioned adjacent patient with range of motion to reach tissue structures of interest (754). Surgical access may be created for a first interventional instrument which may be coupled to a first arm of the robotic system, such as through a transcutaneous surgical port type of access (756). A diagnostic and/or interventional transducer head may be coupled to a second arm of the robotic system and positioned and/or oriented to contact a tissue surface of the patient, such as a skin surface, to provide a coupling for the transducer head to assist in imaging and/or intervening relative to a targeted tissue structure (758). A three-dimensional position and orientation first interventional instrument and transducer head may be determined through the robotic system to which they are both coupled (760). The robotic system may be utilized to conduct an intervention on the targeted tissue structure while simultaneously utilizing the first interventional instrument and the transducer head (762).

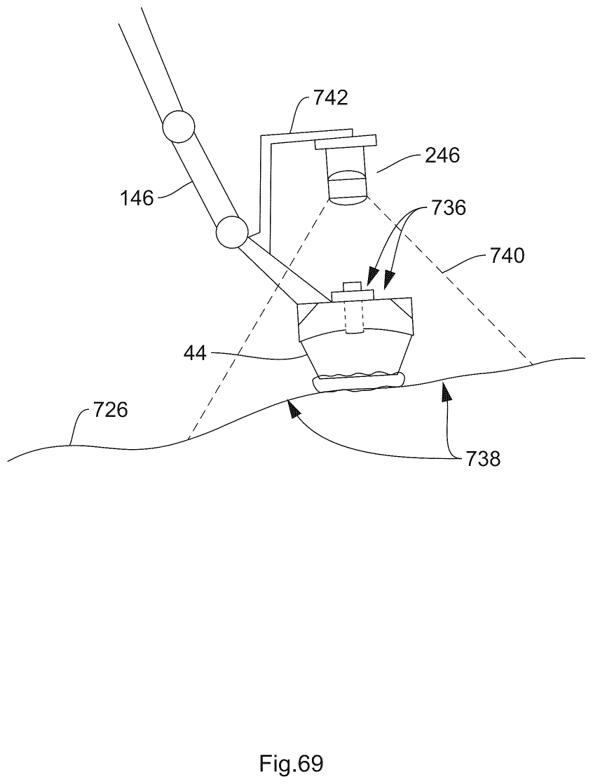

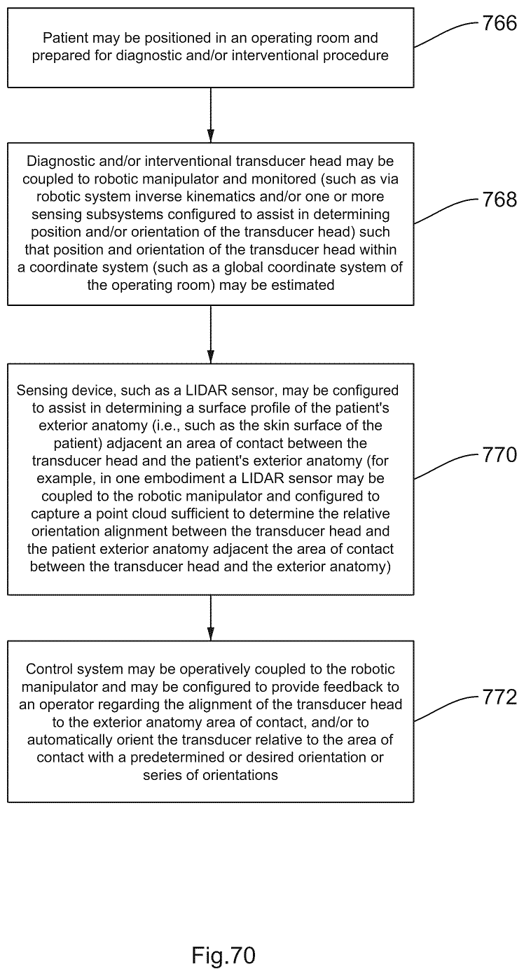

Referring to FIGS. 69 and 70, one or more sensors may be configured to assist in aligning a transducer head (44) relative to a three-dimensional surface profile of the patient which is adjacent the transducer head (44). Referring to FIG. 69, a manipulator assembly (146) is shown positioning a transducer head (44) against a surface of the patient, such as the skin (726) of the patient. A bracket or intercoupling member (742) may be configured to fixedly mount a LIDAR sensor (246) such that a point cloud is created within a captured volume (740) which captures not only points pertaining to three dimensional positions on the back side surfaces (736) of the transducer head (44), which may have known orientations relative to the diagnostic or interventional capabilities of the transducer head (44), but also three dimensional positions of points (i.e., a surface profile) along the surface of the patient (726), and in particular, points (738) along the area surrounding or immediately adjacent to the transducer head (44). Such a configuration, or other related configurations discussed above which may be configured to track the position and/or orientation of the transducer head (44) in space relative to points along the surface of the patient, may be utilized during imaging and/or intervention. For example, referring to FIG. 70, a patient may be positioned in an operating room and prepared for diagnostic and/or interventional procedure (766). A diagnostic and/or interventional transducer head may be coupled to a robotic manipulator and monitored (such as via robotic system inverse kinematics and/or one or more sensing subsystems configured to assist in determining position and/or orientation of the transducer head) such that position and orientation of the transducer head within a coordinate system (such as a global coordinate system of the operating room) may be estimated (768). A sensing device, such as a LIDAR sensor, may be configured to assist in determining a surface profile of the patient's exterior anatomy (i.e., such as the skin surface of the patient) adjacent an area of contact between the transducer head and the patient's exterior anatomy (for example, in one embodiment a LIDAR sensor may be coupled to the robotic manipulator and configured to capture a point cloud sufficient to determine the relative orientation alignment between the transducer head and the patient exterior anatomy adjacent the area of contact between the transducer head and the exterior anatomy) (770). An associated control system may be operatively coupled to the robotic manipulator and may be configured to provide feedback to an operator regarding the alignment of the transducer head to the exterior anatomy area of contact, and/or to automatically orient the transducer relative to the area of contact with a predetermined or desired orientation or series of orientations (772).

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. A minimally invasive system for treating a targeted tissue structure of a patient, comprising:
a. an electromechanical support assembly having a proximal portion and a distal portion;
b. a computing system operatively coupled to the electromechanical support assembly; and
C. a HIFU treatment transducer array coupled to the distal portion of the electromechanical support assembly and operatively coupled to the computing system;
wherein the computing system is configured to operate the electromechanical support assembly to control a position of the transducer assembly relative to the patient such that a treatment focus of the HIFU treatment transducer array is aligned to treat at least a portion of the targeted tissue structure of the patient, and to operate the HIFU treatment transducer array to controllably create a pulsatile wavefront of ultrasound radiation directed at the treatment focus, the pulsatile wavefront configured to produce one or more vapor bubbles within the targeted tissue structure and to controllably produce cavitation of the one of more vapor bubbles such that a controllably lysed portion of the targeted tissue structure is created.

2. The system of claim 1, wherein the electromechanical support assembly comprises a plurality of elongate portions coupled by one or more movable joints.

3. The system of claim 1, wherein the one or more movable joints are coupled to one or more encoders operatively coupled to the computing system and configured to provide inputs to the computing system for determining positions of the one or more movable joints.

4. The system of claim 1, wherein the electromechanical support assembly comprises one or more sensors configured to sense one or more loads within the electromechanical support assembly associated with a physical interface between the HIFU treatment transducer array and the patient.

5. The system of claim 4, wherein the one or more sensors are chosen from the group consisting of: a joint load sensor, a joint torque sensor, a strain gauge, and a deflection gauge.

6. The system of claim 1, further comprising one or more motors operatively coupled to the electromechanical support assembly and configured to apply loads thereto to maintain or change position or orientation of the electromechanical support assembly.

7. The system of claim 6, wherein the electromechanical support assembly comprises a robotic arm.

8. The system of claim 1, wherein the computing system is further configured to operate the electromechanical support assembly to control an orientation of the transducer assembly relative to the patient.

9. The system of claim 1, wherein the electromechanical support assembly is controlled by the computer in response to inputs provided by an operator.

10. The system of claim 9, wherein the inputs provided by the operator are manual electromechanical support assembly movement commands.

11. The system of claim 9, wherein the inputs provided by the operator are commands for the electromechanical support assembly to follow a prescribed set of movements.

12. The system of claim 1, wherein the electromechanical support assembly is controlled by the computer automatically in response to prescribed inputs provided by an operator.

13. The system of claim 1, wherein the HIFU treatment transducer array is operatively coupled to the computing system using a wireless connectivity configuration.

14. The system of claim 1, wherein the HIFU treatment transducer array is operatively coupled to the computing system using a wired connectivity configuration.

15. The system of claim 1, wherein the electromechanical support assembly is operatively coupled to the computing system using a wireless connectivity configuration.

16. The system of claim 1, wherein the electromechanical support assembly is operatively coupled to the computing system using a wired connectivity configuration.

17. The system of claim 1, further comprising an imaging ultrasound transducer having a ultrasound imaging field of view aligned to capture at least a portion of the treatment focus of the HIFU treatment transducer array.

18. The system of claim 17, wherein the HIFU treatment transducer array and the imaging ultrasound transducer are both coupled to the distal portion of the electromechanical support assembly.

19. The system of claim 1, further comprising a delivery interface positioned between the HIFU treatment transducer array and the patient and configured to provide an efficient medium for conducting sound energy between the HIFU treatment transducer array and the patient.

20. The system of claim 19, further comprising a layer of acoustic gel interposed between the delivery interface and the patient and configured to further assist in efficient transmission between the HIFU treatment transducer array and the patient.

21-182. (canceled)
